(19) United States
(12) Patent Application Publication
(10) Pub. No.: US 2005/0203115 A1
(43) Pub. Date: Sep. 15, 2005

(54) Narcotic-NSAID ion pairs

(76) Inventors: Frederick D. Sancilio, Jupiter, FL (US); Grayson W. Stowell, Wilmington, NC (US); Linda B. Whittall, Wilmington, NC (US); David White, Wilmington, NC (US); Robert R. Whittle, Wilmington, NC (US)

Correspondence Address: FOLEY AND LARDNER;SUITE 500, 3000 K STREET NW, WASHINGTON, DC, 20007, US

(21) Appl. No.: 10/796,308

(22) Filed: Mar. 10, 2004

(51) Int. Cl. C07D 489/02; A61K 31/485; A61K 31/192; A61K 31/445; A61K 31/205

(52) U.S. Cl. 514/282; 546/44; 546/232; 514/554; 514/317

(57) ABSTRACT

The present invention provides an ion pair compound of the formula [narcotic]⁺[A]⁻, wherein [narcotic]⁺ represents at least one cation of at least one narcotic agent or one or more stereochemical isomers thereof and [A]⁻ represents at least one anion of at least one NSAID or one or more stereochemical isomers thereof. An example of the ion pair compound is propoxyphene diclofenate. The ion pair compounds, or their pharmaceutical compositions, are useful in methods of treating a wide variety of conditions that indicate analgesics, anti-inflammatory agents, or both. Under the conditions prescribed for their use, the ion pair compounds exhibit poor or complete insolubility but excellent chemical stability in low pH environments, such as those found in the stomach. The ion pair compounds readily dissolve and dissociate in higher pH environments such as the small intestine to release the constituent narcotic and NSAID.

### BACKGROUND OF THE INVENTION

The present invention relates generally to the field of new drug therapies that encompass at least one narcotic and at least one NSAID chemically united as an ion pair.

Many conventional therapeutic regimens rely upon combination therapies, that is, the co-administration of two or more drugs, that often result in certain enhanced therapeutic effects that could not be achieved with a single drug. Positive pharmacodynamic interactions between the drugs in this regard thus fall generally into two broad categories. Two co-administered drugs with similar actions may simply yield an additive effect, essentially evaluated as the sum of the therapeutic effects of the individual drugs. For example, aspirin and codeine are often given together to enhance pain relief.

Second, when two or more drugs are co-administered, one drug may exhibit a synergistic effect on the other drug. That is to say, the combined therapeutic effect of both drugs is greater than the sum of the therapeutic effects ascribed to the individual drugs. A significant advantage in this regard is that lower dosages of one or more of the drugs may result. To illustrate, in a meperidine (a narcotic analgesic) and promethazine (an antihistamine) combination, promethazine enhances the effect of meperidine, thereby allowing the practitioner to administer lower doses of the narcotic.

Combination therapies, as outlined above, present a number of disadvantages. First, as the description implies, combination therapy implicates the administration of at least two drugs, thereby requiring a patient to accept multiple and/or larger dosage forms of the drugs. Such therapies require careful mixing of the drugs to ensure accurate doses of each drug. Scenarios in which the drugs may exhibit negative additive or synergistic effects prescribe additional care to achieve the correct relative dosages and thereby avoid potential adverse effects. Additionally, multiple doses tend to strain patient compliance, particularly among the pediatric and geriatric populations. Thus it would be desirable to co-administer two or more drugs in a single dose in controlled, if not rigorously fixed, proportions.

Second, a substantial subset of drugs is salts or salt prodrugs that, as a consequence of their ionic nature, greatly facilitate their water solubility and resultant bioavailability. The salts necessarily introduce counterions, which although physiologically tolerable, nonetheless represent needless masses of therapeutically irrelevant material that are administered to a patient.

When administered to humans, many drugs do not tolerate the harsh conditions of the stomach, where the lower pH values in the range from about 5.0 (fed) to about 1.7 (fasted) are more than adequate to induce serious chemical degradation of the drugs. Moreover, drugs generally are not absorbed in the stomach but rather in the duodenum (pH=˜4.6, fasted), jejunum (pH=˜4.5-5.5, fed; ˜6.1-6.5, fasted), ileum (pH=˜6.5), and colon (pH=˜8.0) where the pH ranges typically do not facilitate decomposition of the drugs that are acid labile. The foregoing pH ranges may vary from person to person, while other ranges may pertain to other species. A more complete discussion of these pH ranges is given by A. Andeev, Absorption and Drug Development: Solubility, Permeability, and Charge State, Wiley, New York (2003). In this regard, the absence of drug degradation products typically is associated with drugs that are safer for patients. Thus, it is vitally important to ensure the safe passage of drugs through the stomach. Consequently, it is sometimes necessary to increase the dosage of a drug to compensate for the drug's decomposition in the stomach, thus ensuring that a patient receives the therapeutically effective dose of the drug. However, administering greater doses of a drug can present a number of undesirable side effects, such as, for example, irritation or damaging of the stomach lining. This damage of the gastric mucosa may be especially pronounced with the use of non-steroidal anti-inflammatory drugs (NSAIDs). Additionally, many dosage forms incorporate protective coatings and fillers to protect drugs from stomach acid. The resultant increased bulk of the dosage forms is yet another undesirable effect for the reasons mentioned above.

One highly useful subset of drug combinations is NSAIDs and narcotic analgesics (typically opioids). NSAIDs are typically thought to have a mode of action through the arachidonic acid cascade and primarily work at the compartment of injury, resulting in a decrease in the amount of proinflammatory prostaglandins that are produced by cyclooxygenase and lipoxygenase enzymes. On the other hand, analgesics are thought to bind to various types of opioid receptors preventing painful stimuli from reaching the thalamus. It is possible that NSAIDs bind to opioid receptors and that opioid analgesics bind to cyclooxygenases and lipoxygenases, albeit weakly. Together, co-administration of NSAIDs and opioid analgesics have the potential of acting via several mechanisms to ensure the reduction of pain sensation. Additionally, the pairing of an NSAID with a narcotic can result in additive and possibly synergistic analgesic effects and thus minimize the dose of the narcotic and NSAID and their respective side effects. For other reasons as outlined above, however, it may not be necessary or desirable to administer a narcotic and NSAID at full doses to achieve the intended therapeutic effect. Employing lowered narcotic and/or NSAID doses but obtaining the full therapeutic advantages of the narcotic and NSAID would thus present a significant advance over conventional therapies.

Although the mechanism by which NSAIDs cause gastric mucosal damage is not known with certainty, two theories are postulated in the scientific and medical literature. The first model assumes that the protonated acidic NSAID is sufficiently lipophilic to penetrate the cell wall. At the pH interior to the cell, the acidic NSAID loses its proton and becomes trapped in the gastric mucosal cell, causing damage. The second model postulates that a non-selective NSAID binds both isoforms of cyclooxygenase, COX-1 and COX-2. The binding to COX-1 prevents the production of prostaglandins that are thought to repair gastric mucosal damage. Therefore, to prevent gastric mucosal damage, it is desirable to modify the chemical form of the NSAID so that it is not possible for the proton transfer reaction to occur in the stomach. An advantageous result of such modification would result in an NSAID that is insoluble in the acidic pH range of the stomach, but soluble in the neutral to basic pH range of the remainder of the alimentary canal.

In light of the foregoing shortcomings of conventional combination therapies, there exist the needs to co-administer a narcotic and NSAID as a single chemical entity, free of unnecessary counterions, that could achieve the full therapeutic effects of the narcotic and NSAID but avoid the full narcotic dosage of conventional narcotic therapies. Accordingly, the present invention satisfies all of these needs and more by providing an NSAID and narcotic ion pair.

### SUMMARY OF THE INVENTION

The present invention thus provides as one object an ion pair compound according to general formula I:
[narcotic]⁺[A]⁻ (I)

The moiety denoted “[narcotic]⁺” represents at least one cation of at least one narcotic agent or one or more stereochemical isomers thereof, while [A]⁻ represents at least one anion of at least one NSAID or one or more stereochemical isomers thereof. The ion pair compound may also exist as a pharmaceutically acceptable solvate, hydrate, one or more polymorphs, or isotopically labeled version thereof.

The invention provides as another object a pharmaceutical composition comprising a therapeutically effective amount of the inventive ion pair compound and a pharmaceutically acceptable carrier, diluent, excipient, stimulant, or combination thereof. In one embodiment, the pharmaceutical composition comprises an additional NSAID, which can be the same or different as the NSAID represented by A in general formula (I).

Another object of the invention provides a method of treating a condition for which an analgesic is indicated in animals comprising administering to an animal in need of treatment a therapeutically effective amount of the instant ion pair compound. In one alternative, the condition may indicate an anti-inflammatory agent. In another alternative, the condition may indicate both an analgesic and an anti-inflammatory agent. In the foregoing methods, it is possible to employ either the present ion pair compound itself or the present pharmaceutical compositions. All of these combinations are contemplated.

It is yet another object of this invention to provide for a process of preparing the ion pair compound of general formula (I). The process comprises contacting a salt of the formula {[narcotic]⁺}ₓX^{-x} with a salt of the formula [A]⁻B⁺, wherein x is 1, 2, or 3. X is an anion with a charge of −x and B⁺ is a cation.

### BRIEF DESCRIPTION OF THE DRAWINGS

FIG. 1 is an ORTEP of codeine diclofenate monohydrate showing selected atom labels (hydrogen atoms not shown for clarity; 40% thermal ellipsoids).

### DETAILED DESCRIPTION OF THE PREFERRED EMBODIMENTS

The inventors surprisingly discovered that a wide range of narcotic agents, available in their cationic forms, combine readily with NSAIDs in their anionic forms, to yield acid insoluble or acid poorly soluble ion pair compounds of general formula (I) as summarized above. The inventive compounds thus provide a convenient source of two active agents that exhibit remarkable chemical stability to conditions under which the individual free narcotics and/or NSAIDs may decompose or potentially cause gastric mucosal damage.

Ion Pair Compounds

The narcotics that are suitable in the context of this invention are not limited in any particular manner. According to general formula (I), the narcotic should be available in a form that is amenable to the formation of a cation. Most narcotic agents meet this requirement by virtue of their bearing Brönsted acidic moieties, such as amine or amino groups, that can be ionized according to the process of this invention as described more fully below. Additionally, the invention contemplates all stereochemical isomers, where applicable, of the narcotic.

Preferred narcotics in this regard include but are not limited to ketamine, oxycodone, propoxyphene, methadone, hydrocodone, morphine, codeine, fentanyl, meperidine, hydromorphone, oxymorphone, dihydrocodeine, nalbuphine, and buprenorphine. More preferred are meperidine, ketamine, oxycodone, propoxyphene, methadone, hydrocodone, morphine, and codeine. Even more preferred are meperidine, morphine, codeine, methadone, oxycodone, and propoxyphene. The most preferred narcotic is propoxyphene.

In principle, any NSAID is appropriate for use in this invention. According to general formula (I), the NSAID is capable of forming an anion so as to provide charge neutrality for the positively charged narcotic ion. Preferred classes of NSAIDs include but are not limited to non-selective COX inhibitors, selective COX-2 inhibitors, selective COX-1 inhibitors, COX-LOX inhibitors, and PLA₂ inhibitors. The NSAID may be present as one or more stereochemical isomers, where applicable. Exemplary NSAIDs include diclofenac, etodolac, sulindac, alclofenac, fenclofenac, diflunisal, benorylate, fosfosal, salicylic acid, acetylsalicylic acid, ibuprofen, ketoprofen, naproxen, carprofen, fenbufen, flurbiprofen, oxaprozin, suprofen, triaprofenic acid, fenoprofen, indoprofen, piroprofen, flufenamic, mefenamic, meclofenamic, niflumic, salsalate, rolmerin, fentiazac, tilomisole, oxyphenbutazone, phenylbutazone, apazone, feprazone, sudoxicam, isoxicam, tenoxicam, piroxicam, indomethacin, meloxicam, nabumetone, tolmetin, lumiracoxib, and parecoxib. Preferably, the NSAID is diclofenac. In the context of this invention, NSAID is understood to exclude acetaminophen.

The invention thus contemplates all possible combinations of narcotics and NSAIDs according to general formula (I). Exemplary ion pair compounds in this regard include but are not limited to: propoxyphene naproxenate, propoxyphene etodolate, propoxyphene ketoprofenate, propoxyphene sulindate, propoxyphene suprofenate, propoxyphene flurbiprofenate, propoxyphene tolmetinate, propoxyphene fenoprofenate, propoxyphene oxaprozinate, propoxyphene difunisalate, propoxyphene loxoprofenate, ketamine ibuprofenate, ketamine acetylsalicylate, ketamine indomethacinate, ketamine naproxenate, ketamine etodolate, ketamine sulindate, ketamine ketoprofenate, ketamine suprofenate, ketamine flurbiprofenate, ketamine tolmetinate, ketamine fenoprofenate, ketamine oxaprozinate, ketamine difunisalate, ketamine loxoprofenate, ketamine salicylate, ketamine diclofenate, methadone ibuprofenate, methadone acetylsalicylate, methadone salicylate, methadone indomethacinate, methadone naproxenate, methadone etodolate, methadone sulinate, methadone ketoprofenate, methadone suprofenate, methadone flurbiprofenate, methadone tolmetinate, methadone fenoprofenate, methadone oxaprozinate, methadone difunisalate, methadone loxoprofenate, hydrocodone ibuprofenate, hydrocodone acetylsalicylate, hydrocodone salicylate, hydrocodone indomethacinate, hydrocodone naproxenate, hydrocodone etodolate, hydrocodone sulindate, hydrocodone ketoprofenate, hydrocodone suprofenate, hydrocodone flurbiprofenate, hydrocodone tolmetinate, hydrocodone fenoprofenate, hydrocodone oxaprozinate, hydrocodone difunisalate, hydrocodone loxoprofenate, codeine ibuprofenate, codeine acetylsalicylate, codeine salicylate, codeine indomethacinate, codeine naproxenate, codeine etodolate, codeine sulindate, codeine ketoprofenate, codeine suprofenate, codeine flurbiprofenate, codeine tolmetinate, codeine fenoprofenate, codeine oxaprozinate, codeine difunisalate, codeine loxoprofenate, morphine ibuprofenate, morphine acetylsalicylate, morphine salicylate, morphine indomethacinate, morphine naproxenate, morphine etodolate, morphine sulindate, morphine ketoprofenate, morphine suprofenate, morphine flurbiprofenate, morphine tolmetinate, morphine fenoprofenate, morphine oxaprozinate, morphine difunisalate, morphine loxoprofenate, levorphanol ibuprofenate, levorphanol acetylsalicylate, levorphanol salicylate, levorphanol indomethacinate, levorphanol naproxenate, levorphanol etodolate, levorphanol sulindate, levorphanol ketoprofenate, levorphanol suprofenate, levorphanol flurbiprofenate, levorphanol tolmetinate, levorphanol fenoprofenate, levorphanol oxaprozinate, levorphanol difunisalate, levorphanol loxoprofenate, oxycodone ibuprofenate, oxycodone acetylsalicylate, oxycodone salicylate, oxycodone indomethacinate, oxycodone naproxenate, oxycodone etodolate, oxycodone sulindate, oxycodone ketoprofenate, oxycodone suprofenate, oxycodone flurbiprofenate, oxycodone tolmetinate, oxycodone fenoprofenate, oxycodone oxaprozinate, oxycodone difunisalate, oxycodone loxoprofenate; fentanyl naproxenate, fentanyl etodolate, fentanyl ketoprofenate, fentanyl sulindate, fentanyl suprofenate, fentanyl flurbiprofenate, fentanyl tolmetinate, fentanyl fenoprofenate, fentanyl oxaprozinate, fentanyl difunisalate, fentanyl loxoprofenate, meperidine naproxenate, meperidine etodolate, meperidine ketoprofenate, meperidine sulindate, meperidine suprofenate, meperidine flurbiprofenate, meperidine tolmetinate, meperidine fenoprofenate, meperidine oxaprozinate, meperidine difunisalate, meperidine loxoprofenate, hydromorphone naproxenate, hydromorphone etodolate, hydromorphone ketoprofenate, hydromorphone sulindate, hydromorphone suprofenate, hydromorphone flurbiprofenate, hydromorphone tolmetinate, hydromorphone fenoprofenate, hydromorphone oxaprozinate, hydromorphone difinisalate, hydromorphone loxoprofenate, oxymorphone naproxenate, oxymorphone etodolate, oxymorphone ketoprofenate, oxymorphone sulindate, oxymorphone suprofenate, oxymorphone flurbiprofenate, oxymorphone tolmetinate, oxymorphone fenoprofenate, oxymorphone oxaprozinate, oxymorphone difunisalate, oxymorphone loxoprofenate, dihydrocodeine naproxenate, dihydrocodeine etodolate, dihydrocodeine ketoprofenate, dihydrocodeine sulindate, dihydrocodeine suprofenate, dihydrocodeine flurbiprofenate, dihydrocodeine tolmetinate, dihydrocodeine fenoprofenate, dihydrocodeine oxaprozinate, dihydrocodeine difunisalate, and dihydrocodeine loxoprofenate.

Preferred embodiments of the ion pair compound include propoxyphene diclofenate, ketamine diclofenate, methadone diclofenate, hydrocodone diclofenate, codeine diclofenate, propoxyphene salicylate, propoxyphene acetylsalicylate, propoxyphene ibuprofenate, morphine diclofenate, and oxycodone diclofenate. More preferably, the ion pair compound is selected from propoxyphene diclofenate, ketamine diclofenate, methadone diclofenate, hydrocodone diclofenate, codeine diclofenate, morphine diclofenate, and oxycodone diclofenate. The most preferred ion pair compound is propoxyphene diclofenate. In other embodiments, the ion pair compound is propoxyphene salicylate, propoxyphene acetylsalicylate, and propoxyphene ibuprofenate.

In other embodiments, the ion pair compound preferably is propoxyphene lumiracoxibate, ketamine lumiracoxibate, methadone lumiracoxibate, hydrocodone lumiracoxibate, codeine lumiracoxibate, morphine lumiracoxibate, or oxycodone lumiracoxibate. Alternatively, the ion pair compound is selected from the group consisting of propoxyphene parecoxibate, ketamine parecoxibate, methadone parecoxibate, hydrocodone parecoxibate, codeine parecoxibate, morphine parecoxibate, and oxycodone parecoxibate.

The ion pair compound may exist as a pharmaceutically acceptable solvate, hydrate, polymorph, or isotopically labeled version. Pharmaceutically acceptable solvates are those that include, for example, N,N-dimethylformamide (DMF), dimethylsulfoxide (DMSO), acetone, ethers such as diethylether, and alcohols such as methanol and ethanol.

The ion pair compound, when crystalline or micro-crystalline, may exhibit or display a preferred morphology. However, the ion pair compound may exist in one or more other crystal morphologies. Thus, a bulk sample of the compound can include one or more crystal morphologies.

The invention also contemplates isotopically labeled ion pair compounds at one or more atoms. Useful labels in this regard include but are not limited to deuterium, tritium, ¹⁴C, ¹³C, pure ¹²C, ¹¹C, ¹⁷O, ¹⁴N, ¹⁵N, ³⁵Cl, and ³⁷Cl.

The bulk ion pair compound thus may comprise any and all combinations of solvates, hydrates, polymorphs, and isotopically labeled versions.

The inventors were surprised to discover that, relative to an individual narcotic or NSAID, the inventive ion pair compound is decreasingly soluble at lower than neutral pH values, typically becoming completely or at least virtually insoluble at low pH values (e.g., about pH 3 and lower). By contrast, the ion pair compound typically exhibits maximum solubility at pH values of about 7 and higher.

While not wishing to be bound by any particular theory, the inventors believe that the foregoing solubility properties advantageously permit the ion pair compound to exist generally undeterred in the acidic gastric juice of a patient. Under these conditions, the ion pair compound does not solubilize, and thus essentially protects a patient against the risk of the narcotic and/or NSAID decomposing in the stomach, and thereby frequently allows lower dosing. Additionally, the insolubility at low pH avoids, or in the least, minimizes, the potential for gastrointestinal toxicity, such as that of the NSAID irritating or inflaming the stomach lining that is typically observed with NSAIDs generally exhibiting solubility in the acidic stomach environment. Once the ion pair compound passes into the small intestine, where the pH is greater (i.e., about 7), the ion pair compound solubilizes to render the narcotic and NSAID agents as bioavailable therapeutic agents. Thus, the ion pair compound conveniently affords the narcotic and NSAID in one chemical entity that withstands the harsh conditions of the stomach, but readily evolves the drugs in the anatomy where they can be absorbed.

The invention also contemplates a composition comprising a plurality of ion pair compounds, their pharmaceutically acceptable solvates, hydrates, polymorphs, and/or isotopically labeled versions thereof. The composition thus represents the bulk solid that conforms to general formula (I). Any of the foregoing combinations are included in the invention. For example, the composition provides for ion pair compounds that have different narcotic agents and/or NSAIDs. Preferably, however, the composition is homogeneous with respect to the narcotic agent and NSAID. In other embodiments, for example, the composition encompasses one or more polymorphs of the ion pair compound.

Pharmaceutical Composition

The invention also contemplates pharmaceutical compositions that comprise a therapeutically effective amount of at least one ion pair compound according to this invention and a pharmaceutically acceptable carrier, diluent, excipient, stimulant, or combination thereof, the selection of which is known to the skilled artisan. In one embodiment, a solid pharmaceutical composition of the present invention is blended with at least one pharmaceutically acceptable excipient, diluted by an excipient or enclosed within such a carrier that can be in the form of a capsule, sachet, tablet, buccal, lozenge, paper, or other container. When the excipient serves as a diluent, it may be a solid, semi-solid, or liquid material which acts as a vehicle, carrier, or medium for the ion pair compound. Thus, the formulations can be in the form of tablets, pills, powders, elixirs, suspensions, emulsions, solutions, syrups, capsules (such as, for example, soft and hard gelatin capsules), suppositories, lozenges, buccal dosage forms, sterile injectable solutions, and sterile packaged powders.

Examples of suitable excipients include, but are not limited to, starches, gum arabic, calcium silicate, microcrystalline cellulose, polyvinylpyrrolidone, cellulose, water, syrup, and methyl cellulose. The compositions can additionally include lubricating agents such as, for example, talc, magnesium stearate and mineral oil; wetting agents; emulsifying and suspending agents; preserving agents such as methyl- and propyl hydroxybenzoates; sweetening agents; or flavoring agents. Polyols, buffers, and inert fillers may also be used. Examples of polyols include, but are not limited to: mannitol, sorbitol, xylitol, sucrose, maltose, glucose, lactose, dextrose, and the like. Suitable buffers encompass, but are not limited to, phosphate, citrate, tartrate, succinate, and the like. Other inert fillers which may be used encompass those which are known in the art and are useful in the manufacture of various dosage forms. If desired, the solid pharmaceutical compositions may include other components such as bulking agents and/or granulating agents, and the like. The compositions of the invention can be formulated so as to provide normal, sustained, or delayed release of the ion pair compound after administration to the patient by employing procedures well known in the art.

The pharmaceutical composition also may include one or more stimulants, Suitable stimulants in this regard include but are not limited to an effective amount of an amphetamine, such as amphetamine sulfate, dextroamphetamine sulfate, methamphetamine hydrochloride, combinations of amphetamines, derivatives and pharmaceutically salts thereof; pemoline, derivatives and pharmaceutically acceptable salts thereof; methylphenidate, derivatives and pharmaceutically acceptable salts thereof; caffeine, derivatives and pharmaceutically acceptable salts thereof; and centrally acting alpha-1 agonists such as modafinil, epinephrine, norepinephrine, phenylephrine, derivatives thereof and pharmaceutically acceptable salts thereof. The stimulant is intended to reduce or prevent possible dizziness, depression, difficulty in being mobile, drowsiness, lethargy, weakness in the extremities, and orthostatic hypotension associated with administering the ion pair compound of this invention. The preferred stimulant for the treatment of the side effects mentioned above is caffeine.

Some individuals may require a non-amphetamine based stimulant or cannot otherwise receive additional or increased amphetamine doses due to cardiovascular risk concerns. In an alternative embodiment, therefore, a centrally acting alpha-1 agonist, such as modafinil, can be used as a substitute or adjunct for an amphetamine(s), as the stimulant.

A preferred pharmaceutical composition comprises at least one dispersing agent selected from the group consisting of polymer-based dispersing agents and carbohydrate-based dispersing agents and at least one solubilizing agent. The ratio of the ion pair compound to the polymer-based dispersing agent falls in the range from about 3:1 (w/w) to about 1:50 (w/w), while the ratio of the ion pair compound to the carbohydrate-based dispersing agent is from about 3:1 (w/w) to about 1:20 (w/w). Exemplary compositions of this type are described, for example, in U.S. Pat. No. 6,365,180 to Meyer et al. and U.S. Pat. No. 6,287,594 to Wilson et al. Such dispersing agents are well known in the art and include, for example, the polymer-based dispersing agents which include, for example, polyvinylpyrrolidone (PVP; commercially known as Plasdone.RTM.), and the carbohydrate-based dispersing agents such as, for example, hydroxypropylmethylcellulose (HPMC), hydroxypropylcellulose (HPC), and the cyclodextrins. Preferred dispersing agents include PVP K29-32, dextrins, starch, derivatized starch and dextrans, while of the dextrins, derivatized cyclodextrins are especially preferred. Of such cyclodextrins, hydroxypropyl beta.-cyclodextrin and .gamma.-cyclodexrin are especially preferred. The numbers the polymer names refer to the molecular weight of the polymer wherein, for example, PVP K-30 has an average molecular weight of about 30,000, with attendant viscosity characteristics. One or more dispersing agents can be used.

Solubilizing agents suitable for use in the present context are well known in the art and are typically represented by the family of compounds known as polyethylene glycols (PEG) having molecular weights from about 200 to about 8,000. For compositions of the present invention when a liquid is desired for the final formulation or a liquid is to be used to fill soft capsules, preferably soft gelatin capsules, preferred molecular weights range from about 200 to about 600 with PEG 400 being especially preferred. For composition of the present invention when a semi-solid is preferred, especially for filling a hard capsule, preferably a hard gelatin capsule, preferred molecular weight is about 3350 while an especially preferred molecular weight is 3350 plus sufficient 400 molecular weight PEG to improve capsule filling characteristics.

Another solubilizing agent which may be utilized in compositions of the present invention is water, especially purified, and most preferably, deionized. For such compositions, the concentration of water is from about zero percent to about ninety-nine percent (w/w). More particularly for compositions of the present invention to be filled into soft capsules, a maximum water concentration from about 0% to about 5% is preferred, although the concentration of total solubilizing agent may be the full concentration range taught herein.

As used in the present compositions, the concentration of the sum of solubilizing agent utilized, wherein more than one plasticizing agent can be utilized, is from about 0 percent (just greater than zero) to about 99 percent (w/w). The preferred concentration of solubilizing agent in the present compositions is from about 60 percent to about 90 percent (w/w).

One optional component of compositions of the present invention, but which should be used when such compositions are to be filled in soft capsules, is at least one pharmaceutically acceptable and non-toxic plasticizing agent. Such plasticizing agents, which are well known in the pharmaceutical formulation art, include, for example, glycerin, propylene glycol, and sorbitol. Such commercially available plasticizers can be prepared to include more than one plasticizing agent component, but the preferred plasticizing agent for the present compositions is glycerin. In addition to its use as a plasticizing agent, propylene glycol can be used as a solubilizing agent when used alone or in combination with another solubilizing agent as taught herein.

As used in the present invention, the concentration of the sum of plasticizing agent utilized, wherein more than one plasticizing agent can be utilized, is from about zero percent (just greater than zero) to about 75 percent (w/w). The preferred concentration of plasticizing agent is from about zero percent (0%) to about fifty percent (50%), and an especially preferred concentration in a range from about one percent (1%) to about thirty percent (30%). When the compositions of the present invention are used to fill soft capsules, the preferred concentration of such plasticizing agent is from about 5 percent to about 10 percent (w/w). Such plasticizers are especially useful with soft capsule preparations because, without which, such capsules tend to harden and lose their beneficial properties by potentially cracking or becoming brittle.

Another optional component of the present compositions, which is a preferred component, is at least one pharmaceutically acceptable, non-toxic, surfactant, preferably a non-ionic surfactant. Such surfactants are well known in the pharmaceutical formulation art and include readily available surfactants having a concentration from about zero percent to about 90 percent such as, for example, macro gel esters (Labrafils), Tandem 522.RTM., Span 80.RTM., Gelucieres.RTM. such as, for example, tocopherol polyethylene glycol 1000 succinate, polysorbate 20, and polysorbate 80. Of these, polysorbate 20 and polysorbate 80 are preferred.

As used in the present invention, the concentration of the sum of non-ionic surfactant utilized, wherein more than one such surfactant can be utilized, is from about zero percent to about 10 percent (w/w), with a range from about 1 percent to about 5 percent (w/w) being preferred. An especially preferred concentration is about 3 percent (w/w).

In the event that the foregoing compositions are to be used for parenteral administration, such a formulation typically comprises sterile aqueous and non-aqueous injection solutions comprising the ion pair compound, for which preparations are preferably isotonic with the blood of the intended recipient. These preparations may contain anti-oxidants, buffers, bacteriostats, and solutes which render the formulation isotonic with the blood of the intended recipient. Aqueous and non-aqueous sterile suspensions may include suspending agents and thickening agents.

The compositions may be presented in unit-dose or multi-dose containers, for example sealed ampules and vials. Extemporaneous injection solutions and suspensions may be prepared from sterile powders, granules and tablets of the kind previously described.

In preferred embodiments of the invention, the composition may be made into the form of dosage units for oral administration. The ion pair compound may be mixed with a solid, pulverant carrier such as, for example, lactose, saccharose, sorbitol, mannitol, starch, amylopectin, cellulose derivatives or gelatin, as well as with an antifriction agent such as, for example, magnesium stearate, calcium stearate, and polyethylene glycol waxes. The mixture is then pressed into tablets. If coated tablets are desired, the above prepared core may be coated with a concentrated solution of sugar, which may contain gum arabic, gelatin, talc, titanium dioxide, or with a lacquer dissolved in volatile organic solvent or mixture of solvents. To this coating, various dyes may be added in order to distinguish among tablets with different active compounds or with different amounts of the active compound present.

Soft capsules also may be prepared in which capsules contain a mixture of the ion pair compound and vegetable oil or non-aqueous, water miscible materials such as, for example, polyethylene glycol and the like. Hard capsules may contain granules of the ion pair compound in combination with a solid, pulverulent carrier, such as, for example, lactose, saccharose, sorbitol, mannitol, potato starch, corn starch, amylopectin, cellulose derivatives, or gelatin.

Dosage units for rectal administration may be prepared in the form of suppositories which may contain the ion pair compound in a mixture with a neutral fat base, or they may be prepared in the form of gelatin-rectal capsules which contain the active substance in a mixture with a vegetable oil or paraffin oil.

Liquid preparations for oral administration may be prepared in the form of syrups or suspensions, e.g., solutions containing an ion pair compound, sugar, and a mixture of ethanol, water, glycerol, and propylene glycol. If desired, such liquid preparations may contain coloring agents, flavoring agents, and saccharin. Thickening agents such as carboxymethylcellulose may also be used.

Tablets for oral use are typically prepared in the following manner, although other techniques may be employed. The solid substances are gently ground or sieved to a desired particle size, and the binding agent is homogenized and suspended in a suitable solvent. The ion pair compound and auxiliary agents are mixed with the binding agent solution. The resulting mixture is moistened to form a uniform suspension. The moistening typically causes the particles to aggregate slightly, and the resulting mass is gently pressed through a stainless steel sieve having a desired size. The layers of the mixture are then dried in controlled drying units for determined length of time to achieve a desired particle size and consistency. The granules of the dried mixture are gently sieved to remove any powder. To this mixture, disintegrating, anti-friction, and anti-adhesive agents are added. Finally, the mixture is pressed into tablets using a machine with the appropriate punches and dies to obtain the desired tablet size. The operating parameters of the machine may be selected by the skilled artisan.

Typically, preparation of lozenge and buccal dosage forms are prepared by methods known to one of ordinary skill in the art.

In other embodiments, the ion pair compound may be present in a core surrounded by one or more layers including, for example, an enteric coating layer with or without a protective sub-coating as known to the ordinarily skilled artisan relative to pharmaceutical formulations.

The final dosage form encompassing the above embodiments may be either an enteric coated tablet or capsule or in the case of enteric coated pellets, pellets dispensed in hard capsules or sachets or pellets formulated into tablets. It is desirable for long term stability during storage that the water content of the final dosage form containing the ion pair compound (enteric coated tablets, capsules or pellets) be kept low. As a consequence, the final package containing hard capsules filled with enteric coated pellets preferably also contain a desiccant, which reduces the water content of the capsule shell to a level where the water content of the enteric coated pellets filled in the capsules does not exceed a certain level.

Accordingly, the ion pair compounds and compositions of the present invention are preferably formulated in a unit dosage form, each dosage containing from about 5 mg to about 200 mg, and more preferably the amount set forth herein. The term “unit dosage form” refers to physically discrete units, such as capsules or tablets suitable as unitary dosages for human patients and other mammals, each unit containing a predetermined quantity of one or more ion pair compound(s) calculated to produce the desired therapeutic effect, in association with at least one pharmaceutically acceptable carrier, diluent, excipient, or combination thereof. Generally, preferred dosages of the ion pair compounds in such unit dosage forms are from about 5 mg to about 15 mg, about 20 mg to about 30 mg, about 40 mg to about 60 mg, and about 65 mg to about 120 mg, especially 12 mg, 25 mg, and 48 mg, and 95 mg per dosage unit.

Additional NSAID or Narcotic

As mentioned above, an advantage of the inventive ion pair compound is the ability to dose a narcotic and NSAID in one chemical entity to a patient. However, in some circumstances, the stoichiometry between the narcotic and NSAID (e.g., 1:1) may not accommodate the prescribed overall dosage of the NSAID. Therefore, certain embodiments of the pharmaceutical composition comprise a therapetucially effective amount of an additional NSAID, or a pharmaceutically acceptable salt, solvate, hydrate, polymorph, or isotopically labeled version thereof. In these embodiments, the additional NSAID may be the same or different from the NSAID represented by “A” in general formula (I).

Alternatively, some relative dosage requirements for a given NSAID and narcotic warrant adding to the pharmaceutical composition an additional and therapeutically effective amount of a narcotic or a pharmaceutically acceptable salt, solvate, hydrate, polymorph, or isotopically labeled version thereof. In this regard, the narcotic need not be the same as the narcotic represented by general formula (I). Preferably, however, the additional narcotic is the same as the narcotic in general formula (I).

A preferred composition in this context comprises propoxyphene diclofenate. In this embodiment, the additional NSAID may be present as diclofenac free acid or a pharmaceutically acceptable salt thereof. Exemplary salts in this regard include the sodium and potassium salts of diclofenac.

In preferred embodiments, the additional NSAID or narcotic may be contained in an external or enteric coating as described above. The additional NSAID or narcotic thus is available for immediate, slow, delayed, sustained, pseudo-first order, pseudo-zero order, timed, controlled release, or combinations thereof. The additional NSAID or narcotic agent can be applied to the surface of a dosage form according to common methods that are known to those of ordinary skill such as applying to its surface solids in solution or suspension through the use of a sprayer that spreads them uniformly over the core or by employing nucleated compression or other suitable methods known to those of ordinary skill in the art. The external coat can comprise poly(vinyl pyrrolidone) (PVP) and poly(ethylene glycol) (PEG) and can further comprise materials such as, by way of example and without limitation, hydroxypropyl methylcellulose (HPMC), ethylcellulose (EC), hydroxyethylcellulose (HEC), sodium carboxymethyl-cellulose (CMC), dimethylaminoethyl methacrylate-methacrylic acid ester copolymer, ethylacrylate-methylmethacrylate copolymer (GA-MMA), C-5 or 60 SH-50 (Shin-Etsu Chemical Corp.) and combinations thereof. The external coat can also comprise dissolution aids, stability modifiers, and bioabsorption enhancers.

The amount of the additional NSAID or narcotic depends upon the individual NSAID or narcotic and its dosage requirements that are known to the person of skill in the art.

Methods of Treatment

The invention also provides methods of treating a condition in an animal in need of treatment comprising administering to the animal a therapeutically effective amount of the ion pair compound or a pharmaceutical composition as described above. In some embodiments, the condition is one for which is indicated an analgesic. In other embodiments, an anti-inflammatory agent is indicated. In still other embodiments, the condition indicates both an analgesic and anti-inflammatory agent.

Preferably, the animal suffering from the condition is a mammal. More preferably, the mammal is a human being.

As used herein, the term “treatment” or “treating” contemplates partial or complete inhibition of the stated condition or disease state when an ion pair compound or its pharmaceutical composition is administered prophylactically or following the onset of the condition for which the compound or composition is administered. For the purposes of this invention, the term “prophylaxis” refers to the administration of the ion pair compound to an animal to protect the animal from any of the conditions set forth herein.

The inventive ion pair compound may treat a number of conditions including arthritic disorders, gastrointestinal conditions, inflammatory conditions, pulmonary inflammation, opthalmic diseases, central nervous systems disorders, pain, fever, inflammation-related cardiovascular disorders, angiogenesis-related disorders, benign and malignant tumors, adenomatous polyps, fibrosis which occurs with radiation treatment, endometriosis, osteoporosis, dysmenorrhea, premature labor, asthma, eosinophil-related disorders, pyrexia, bone resorption, nephrotoxicity, hypotension, arthrosis, joint stiffness, kidney disease, liver disease, acute mastitis, diarrhea, colonic adenomas, bronchitis, allergic neuritis, cytomegalovirus infectivity, apoptosis, HIV-induced apoptosis, lumbago, psoriasis, eczema, acne, burns, dermatitis, ultraviolet radiation damage, allergic rhinitis, respiratory distress syndrome, and endotoxin shock syndrome.

The invention is particularly effective in the treatment of arthritic disorders. These include but are not limited to rheumatoid arthritis, osteoarthritis, and acute gouty arthritis.

Other conditions against which the invention is effective include primary dysmenorrhea, anklosing spondylitis, and inflammatory disorders. Exemplary inflammatory disorders in this context include tendonitis and bursitis.

By virtue of incorporating a narcotic and NSAID, the ion pair compound is also highly effective in the treatment of many types of pain. Certain types of pain contemplated by this invention arise from pre-operative, post-operative, and both pre- and post-operative procedures. Examples of pain that are treated by this invention thus include anogenital, minor arthritic, dental, topical, associated with an upper respiratory infection, general, joint, menstrual, mild, mild to moderate, acute musculo-skeletal, moderate to moderately severe, moderate to severe, muscular, neurogenic, obstetrical, ocular, oral mucosal and gingival, post operative, pre-operative, pre- and post-operative, severe, short term, urinary tract, and pain associated with gastric hyperacidity.

Typical doses of the ion pair compound will depend upon various factors such as, for example, the individual requirement of each patient, the route of administration, and the disease. One advantage of the ion pair compound in this regard is that the dosage strength of the compound may closely match the dosages of the individual narcotic and NSAID, which are well-known to the person of skill in the art. An attending physician may adjust the dosage rate based on these and other criteria if he or she so desires. As an example, a suitable oral dosage form may encompass from about 5 to about 1000 mg total daily dose, typically administered in one single dose or equally divided doses. A more preferred range is from about 15 mg to about 600 mg total daily dose, and a most preferred range is from about 30 mg to about 300 mg total daily dose. Additionally, the ion pair compound(s) may be administered as a suspension, and, as an example, the daily doses set forth above may be employed. In one embodiment, the ion pair compound(s) may be added in appropriate amounts to a liquid such that the resultant suspension comprises, for example, from about 0.1 mg/mL to about 10 mg/mL of the ion pair compound(s). It should be noted that daily doses other than those described above may be administered to a subject, as appreciated by an attending physician.

Process for Preparing

The invention also provides a process for preparing the ion pair compound represented by general formula (I). In general, as described summarily above, the narcotic is introduced as a cation according to the formula {[narcotic]⁺}ₓX^{x-}. In this regard, x is 1, 2, or 3, while X is a charge-balancing anion with an overall charge of −x. Anions represented by X include but are not limited to halides, such as chloride, bromide, and iodide; sulfate; nitrate; and phosphate. X may also represent one of many organic anions, such as carboxylates and organic sulfates or sulfonates. Exemplary anions in this regard include napsylate, terephthalate, citrate, bitartrate, and tartrate. Additional anions include the conjugate bases of the acids that are described below. It is thus possible to employ narcotic starting materials that incorporate multiple narcotic cations. Many narcotics are available commercially as salts represented by {[narcotic]⁺}ₓX^{x-}. Typically, x is 1. Examples in this regard include hydrohalogen acid salts, such as hydrochloride salts.

Where the narcotic is not available as a salt, acid addition salts of the narcotic may be prepared straightforwardly. Acids suitable for making such salts include but are not limited to hydrohalogen acids, sulfuric, phosphoric, nitric, and perchloric acids; aliphatic, alicyclic, aromatic, heterocyclic carboxy or sulfonic acids, such as formic, acetic, propionic, succinic, glycolic, lactic, malic, tartaric, citric, ascorbic, maleic, hydroxymaleic, pyruvic, phenylacetic, benzoic, p-aminobenzoic, antranilic, p-hydroxybenzoic, salicylic or p-aminosalicylic acid, embonic, methanesulfonic, ethanesulfonic, hydroxyethanesulfonic, ethylenesulfonic, halogenbenzenesulfonic, toluenesulfonic, naphtylsulfonic or sulfanilic acids; methionine, tryptophane, lysine or arginine.

The narcotic salt of the formula {[narcotic]⁺}ₓX^{x-} is contacted with an NSAID salt of the formula [A]⁻[B]⁺, where B represents the charge balancing cation for the negatively charged NSAID. Many NSAIDs already are available commercially as salts of this formula. For example, a preferred salt in this regard is sodium diclofenac. Salts of the formula [A]⁻[B]⁺ can be prepared where an NSAID is not readily available as a salt. Such salts typically are prepared from an NSAID that bears at least one “acidic” proton. The proton may be removed, for example, by a type of base that allows for the formation of an anionic species of the NSAID countered by the cation. Some embodiments encompass polar, protic environments, in which alkali or alkaline metal hydroxide or alkaline metal alkoxides present are effective in an alcohol or in mixed organic solvent such as a 2-butanone-toluene mixture.

The narcotic and NSAID salts, as set forth above, may be combined in a variety of ways to yield the present ion pair compound. In one embodiment, the compounds of formulae {[narcotic]⁺}ₓX^{x-} and [A]⁻[B]⁺ thus are dissolved in separate volumes of the same solvent or in different solvents. When combined, the resultant solution thus yields the ion pair compound of general formula (I) and the stoichiometric amounts of the undesired counterions X^{x-} and B⁺. The solvent or solvent mixture can be selected such that the ion pair compound precipitates when the separate volumes of {[narcotic]⁺}ₓX^{x-} and [A]⁻[B]⁺ are combined, thereby allowing the easy isolation of the ion pair compound. Alternatively, the ion pair compound is soluble in the combined volumes of solvent or different solvents. In this scenario, the solvent(s) may be removed to yield the ion pair compound, which can then be purified according to standard purification techniques known to those who are skilled in the art.

In another embodiment, the compounds according to formulae {[narcotic]⁺}ₓX^{x-} and [A]⁻[B]⁺ are contacted effectively with each other on a cation exchange medium, such as on a cation exchange chromatography column. In this embodiment, the [narcotic]⁺ is retained on the cation exchange column when {[narcotic]⁺}ₓX^{x-} is introduced. When [A]⁻[B]⁺ is passed through the cation exchange column, the [B]⁺ is retained and the desired [narcotic]⁺[A]⁻ ion pair compound is recovered and subsequently isolated according to conventional techniques in the art.

The following examples are intended to further describe the invention by way of illustration, and thus should not be construed as limiting the scope of the invention in any way.

All publicly available documents cited in this description are incorporated by reference as if fully set forth herein.

### EXAMPLE 1

### Preparation of d-Propoxyphene Diclofenate from Sodium Diclofenac and d-Propoxyphene Napsylate Hydrate

Sodium {2-[(2,6-dichlorophenyl)amino]}phenylacetate (referred to herein as sodium diclofenac) (0.3283 g, 1.032 mmol) was dissolved in methanol (25 mL) to which was added a methanol solution (25 mL) of (2S,3R)-(+)-4-(dimethylamino)-3-methyl-1,2-diphenyl-2-butanol propionate (referred to herein as d-propoxyphene) napsylate hydrate (0.5633 g, 0.996 mmol). The two solutions were mixed well and the methanol removed over several hours by evaporation under an air purge. An oily material, which contained a white residue, was formed. Water (100 mL) was added to the oily material and solution formation enhanced by means of sonication (5 minutes). The aqueous supernatant was decanted and the residual oily material dried under reduced pressure. Methanol (25 mL) was added to dissolve the oily material, and any solid material removed by filtration through 0.45-μm polytetrafluoroethylene (PTFE). Solvent was removed by evaporation under a stream of nitrogen, which resulted in the formation of an oily material. This oily material was dissolved in dichloromethane (30 mL), which resulted in the formation of a precipitate, which was removed by filtration through 0.45-μm PTFE. The dichloromethane was removed by evaporation under nitrogen to produce the desired product as an oil or glass. The product was characterized by means of Fourier Transform Infra-Red Spectroscopy (FTIR). Representative bands are listed in the Table 1.

TABLE 1
Observed bands for FTIR spectrum from Example 1.

| Band (cm⁻¹) | Intensity |
|---|---|
| 3243 | weak, broad |
| 3061 | weak |
| 3032 | weak |
| 2977 | moderate |
| 2942 | moderate |
| 2819 | weak |
| 2764 | weak |
| 2819 | weak |
| 2764 | weak |
| 1733 | strong |
| 1603 | moderate |
| 1577 | strong |
| 1561 | moderate |
| 1498 | strong |
| 1453 | very strong |
| 1351 | moderate |
| 1280 | moderate |
| 1178 | strong |
| 1081 | moderate |
| 1020 | moderate |
| 773 | moderate |
| 745 | moderate |
| 704 | strong |

### EXAMPLE 2

### Preparation of d-Propoxyphene Diclofenate from Sodium Diclofenac and d-Propoxyphene Napsylate Hydrate by Ion Exchange Chromatography

d-Propoxyphene napsylate hydrate (1.8228 g, 3.222 mmol) in methanol (60 mL) was placed on a Varian MegaBond Elut strong cation exchange column (SCX), which was pre-treated with methanol. A solution of sodium diclofenac (1.0283 g, 3.232 mmol) in methanol (3 mL) was added to the column and the product eluted with excess methanol. The methanol solution was concentrated by rotary evaporation, reconstituted in dichloromethane (30 mL) with sufficient methanol to dissolve the material The solution was placed in a nitrogen cabinet for approximately 12 hours. The sample was removed from the nitrogen cabinet and the remaining solvent removed by rotary evaporation, which resulted in the formation of a white solid. The solid was dissolved in methanol and placed in the nitrogen cabinet for approximately 48 hours to remove the solvent by evaporation. The resulting viscous oil containing crystalline plates was washed with acetone to dissolve the oil. The acetone solution was decanted from the insoluble crystalline plates into a small beaker. A small amount of diethyl ether was added to the acetone solution to induce recrystallization and the solution placed in a nitrogen cabinet to remove the solvent by evaporation, which resulted in the formation of an oily material. Multiple attempts at recrystallization of the oily material using different solvents and conditions did not yield a crystalline product. A small portion of the oily material from this series of attempts was dissolved in a solution of methanol and water (5:1) resulting in the formation of a white precipitate. The precipitate was separated by filtration through Whatman #4 filter paper and dried under nitrogen. The product was characterized by FTIR spectroscopy. Representative bands are listed in the Table 2.

TABLE 2
Observed bands for FTIR spectrum from Example 2.

| Band (cm⁻¹) | Intensity |
|---|---|
| 3378 | weak, broad |
| 3059 | weak |
| 3031 | weak |
| 2976 | weak |
| 2923 | weak |
| 2852 | weak |
| 1734 | strong |
| 1603 | moderate |
| 1577 | strong |
| 1560 | moderate |
| 1497 | strong |
| 1454 | very strong |
| 1384 | weak |
| 1189 | strong |
| 1091 | moderate |
| 1031 | moderate |
| 972 | weak |
| 867 | weak |
| 828 | weak |
| 774 | moderate |
| 748 | moderate |
| 705 | moderate |
| 676 | moderate |
| 648 | weak |
| 624 | weak |
| 569 | weak |

### EXAMPLE 3

### Preparation of d-Propoxyphene Diclofenate from Sodium Diclofenac and d-Propoxyphene Hydrochloride

Sodium diclofenac (0.9543 g, 3.000 mmol) in water (200 mL) was placed in a 500 mL Erlenmeyer flask. d-Propoxyphene hydrochloride (1.1219 g, 2.984 mmol) in water (200 mL) was added to the diclofenate solution resulting in the formation of a white precipitate. After mixing, the water was removed by decantation and the residual solid dissolved in an appropriate amount of diethyl ether and transferred to a 200 mL round bottom flask. The solvent was removed by rotary evaporation and the product dried under vacuum. The resulting product was a white solid.

### EXAMPLE 4

### Preparation of d-Propoxyphene Diclofenate from Potassium Diclofenac and d-Propoxyphene Hydrochloride

Potassium diclofenac (0.3828 g, 1.145 mmol) was dissolved in water (100 mL) and placed in a 250 mL round bottom flask. An aqueous solution of d-propoxyphene hydrochloride (0.4384 g, 1.166 mmol in 50 mL of water) was added to the round bottom flask with stirring, which resulted in the formation of a white precipitate. The water was decanted a small portion of the solid was analyzed by means of FTIR. Representative bands are listed in Table 3. The residual solid was dissolved in toluene (80 mL) and transferred to a separatory funnel. The organic layer was washed with water (3×40 mL), dried (MgSO₄), and the resulting solid separated by filtration through a 0.45-μm polyvinylidene fluoride (PVDF) filter. The solvent was removed by rotary evaporation, which resulted in an oily material. The product was assayed by supercritical fluid chromatography (SFC; 101.10% propoxyphene; 99.6% diclofenate).

TABLE 3
Observed bands for FTIR spectrum from Example 4.

| Band (cm⁻¹) | Intensity |
|---|---|
| 3243 | weak, broad |
| 3061 | weak |
| 3031 | weak |
| 2976 | moderate |
| 2940 | moderate |
| 2818 | weak |
| 2763 | weak |
| 1733 | strong |
| 1577 | moderate |
| 1506 | strong |
| 1497 | strong |
| 1452 | very strong |
| 1350 | moderate |
| 1281 | moderate |
| 1177 | strong |
| 1080 | moderate |
| 1019 | moderate |
| 865 | weak |
| 773 | moderate |
| 746 | moderate |
| 704 | strong |
| 646 | weak |
| 579 | weak |
| 531 | weak |

### EXAMPLE 5

### Preparation of d-Propoxyphene Diclofenate from Potassium Diclofenate and d-Propoxyphene Hydrochloride

Potassium diclofenate (3.3761 g, 10.101 mmol) in water (600 mL) was placed in a 1 L Erlenmeyer flask. d-Propoxyphene hydrochloride (3.7884 g, 10.077 mmol) in water (100 mL) was added to the diclofenate solution forming a white precipitate. After mixing for 15 minutes, the contents of the 1 L Erlenmeyer flask were transferred to a separatory funnel with the aid of a small portion of diethyl ether. Diethyl ether (250 mL) was added to the separatory funnel and any remaining precipitate was dissolved with shaking. The organic and aqueous layers were separated and the aqueous layer washed with additional diethyl ether (2×250 mL) to extract any remaining product. The organic layers were combined and the solvent removed by rotary evaporation. The resulting oily material was placed under reduced pressure to form a white solid. After trying to constant weight, the white solid was characterized by supercritical fluid chromatography (SFC): 100.7% propoxyphene; 98.7% diclofenate; elemental analysis (CHN): Expected: 68.03% C, 6.34% H, 4.41% N, Obtained; 67.90% C, 6.22% H, 42% N; and differential scanning calorimetry (DSC): Glass transition (T_{g}): 30.1° C.; degradation 189° C.).

### EXAMPLE 6

### Preparation of d-Propoxyphene Diclofenate from Potassium Diclofenate and d-Propoxyphene Hydrochloride

Aqueous solutions of potassium diclofenac (5.0227 g, 15.027 mmol in 1 L of water) and d-propoxyphene hydrochloride (5.6627 g, 15.063 mmol in 300 mL of water) were combined into a 2 L round bottom flask. A white precipitate formed and the solution was stirred for 30 minutes. An appropriate amount of diethyl ether was added to the 2 L round bottom flask containing the aqueous solution and precipitate. Upon addition of the diethyl ether, the precipitate dissolved with stirring. The resulting aqueous/organic solution was transferred to a separatory funnel in several portions and the organic and aqueous layers separated. The organic layers were combined, the diethyl ether removed by rotary evaporation and the product placed under vacuum. The resulting white solid was assayed by SFC: propoxyphene 100.2%; diclofenac 99.6%.

### EXAMPLE 7

### Preparation of d-Propoxyphene Diclofenate from Potassium Diclofenac and d-Propoxyphene Hydrochloride

Potassium diclofenac (8.3559 g, 25.000 mmol) was dissolved in water (800 mL). An aqueous solution of propoxyphene hydrochloride (9.3889 g, 24.974 mmol in 500 mL of water) was added to the diclofenac solution in a 4 L Erlenmeyer flask. A white precipitate formed and the solution was stirred for 30 minutes. An appropriate amount of diethyl ether was added to the 4 L Erlenmeyer flask containing the aqueous solution and precipitate. Upon addition of the diethyl ether, the precipitate dissolved with stirring. The resulting aqueous/organic solution was transferred to a separatory funnel in several portions and the organic and aqueous layers separated. The organic layers were combined, the diethyl ether removed by rotary evaporation and the product placed under vacuum. The resulting white solid was characterized by SFC: 98.9% propoxyphene; 99.6% diclofenac; and Nuclear Magnetic Resonance (NMR) Spectroscopy. Resonances for the ¹H and ¹³C NMR spectra obtained in d₆-dimethylsulfoxide (DMSO) solution are listed in Tables 4a and 4b, respectively.

TABLE 4a
Observed resonances for the ¹H NMR spectrum from Example 7 obtained
in d₆-DMSO solution.

| Resonance (ppm) | Multiplicity* | Number of Protons |
|---|---|---|
| 7.62 | bs | 1 |
| 7.36-7.50 | d | 2 |
| 7.38 | s, d | 4 |
| 7.27-7.34 | m | 1 |
| 7.15-7.23 | m | 5 |
| 7.04 | t | 1 |
| 6.99-7.01 | s, d | 2 |
| 6.84 | t | 1 |
| 6.29 | dd | 1 |
| 3.80 | q | 2 |
| 3.67 | s | 2 |
| 2.63 | m | 1 |
| 2.40 | dd | 1 |
| 2.27 | q | 2 |
| 2.06 | s | 6 |
| 1.60 | t | 1 |
| 0.94-1.09 | t, d | 6 |

*s - singlet, d - doublet, dd - doublet of doublets, m - multiplet, b - broad, t - triplet, q - quartet.

TABLE 4b
Observed resonances for the ¹³C NMR spectrum from Example 7 obtained
from d₆-DMSO solution
Resonance (ppm)

173.67
172.57
142.68
139.35
137.22
136.54
130.75
129.96
129.80
129.13
127.81
127.47
127.21
126.82
126.38
124.55
120.64
115.89
87.62
60.75
45.31
38.59
36.43
28.32
14.31
8.99

### EXAMPLE 8

### Preparation of rac-Ketamine Diclofenate from Sodium Diclofenac and rac-Ketamine Hydrochloride

Aqueous solutions of sodium diclofenac (0.6378 g, 2.005 mmol in 150 mL of water) and (±)-2-(2-chlorophenyl)-2-(methylamino)cyclohexanone (referred to herein as rac-ketamine) hydrochloride (0.5427 g, 1.979 mmol in 50 mL of water) were combined into a 250 mL Erlenmeyer flask. A white precipitate formed and the solution was stirred for 15 minutes. The solid material was separated by filtration through a 0.45-μm polyvinylidene fluoride (PVDF) filter and the filter cake dissolved in methanol (25 mL). The methanol solution was removed by evaporation under nitrogen and the resulting oily material transferred to a round bottom flask using a small amount of diethyl ether. The diethyl ether was removed by rotary evaporation forming a white solid. The flask placed under vacuum to obtain a white solid product. The product was characterized by elemental analysis: Expected: 60.74% C, 5.10% H, 5.25% N, Obtained: 59.88% C, 4.87% H, 5.14% N; DSC: T_{g}: 40.3° C.; ¹H and ¹³C NMR, FTIR, and FT-Raman Spectroscopy (FT-Raman). Representative bands observed in the FTIR and FT-Raman spectra are listed in the Tables 5a and 5b, respectively. Resonances for the ¹H and ¹³C NMR spectra are listed in Tables 6a and 6b, respectively.

TABLE 5a
Observed bands for FTIR spectrum from Example 8.

| Band (cm⁻¹) | Intensity |
|---|---|
| 2942 | weak |
| 1724 | moderate |
| 1578 | moderate |
| 1505 | strong |
| 1452 | very strong |
| 1375 | weak |
| 1304 | moderate |
| 1229 | weak |
| 1196 | weak |
| 1149 | weak |
| 1113 | weak |
| 1088 | weak |
| 1049 | weak |
| 947 | weak |
| 892 | weak |
| 867 | weak |
| 747 | strong |
| 715 | moderate |

TABLE 5b
Observed bands for FT-Raman spectrum from Example 8.

| Band (cm⁻¹) | Intensity |
|---|---|
| 3070 | very strong |
| 2987 | moderate |
| 2969 | moderate |
| 2869 | weak |
| 1725 | weak |
| 1603 | strong |
| 1587 | strong |
| 1577 | very strong |
| 1448 | weak |
| 1277 | weak |
| 1250 | weak |
| 1234 | moderate |
| 1194 | weak |
| 1159 | weak |
| 1093 | weak |
| 1070 | moderate |
| 1047 | strong |
| 891 | weak |
| 837 | weak |
| 717 | weak |
| 653 | weak |
| 605 | weak |
| 517 | weak |
| 443 | moderate |
| 404 | weak |
| 363 | weak |
| 316 | weak |
| 216 | weak |
| 177 | weak |

TABLE 6a
Observed resonances for the ¹H NMR spectrum from Example 8 obtained
from d₆-DMSO solution.

| Resonance (ppm) | Multiplicity* | Number of Protons |
|---|---|---|
| 7.60 | dd | 1 |
| 7.52 | d | 2 |
| 7.26-7.39 | m | 3 |
| 7.17-7.22 | d, t | 2 |
| 7.06 | t | 1 |
| 6.86 | t | 1 |
| 6.29 | dd | 1 |
| 3.70 | s | 2 |
| 2.28-2.53 | m | 3 |
| 1.96 | s | 3 |
| 1.61-1.94 | m | 3 |

*s - singlet, d - doublet, dd - doublet of doublets, m - multiplet, b - broad, t - triplet, q - quartet.

TABLE 6b
Observed resonances for the ¹³C NMR spectrum from Example 8 obtained
from d₆-DMSO solution.
Resonance (ppm)

204.55
173.34
142.65
139.72
137.12
132.44
130.85
130.55
129.99
129.15
128.43
127.46
126.76
125.51
123.98
120.73
115.94
68.63
38.29
37.94
37.85
29.33
25.01
20.43

### EXAMPLE 9

### Preparation of rac-Methadone Diclofenate from Sodium Diclofenac and rac-Methadone Hydrochloride

Sodium diclofenac (0.6400 g, 2.012 mmol) was dissolved in water (150 mL) and placed in a 250 mL Erlenmeyer flask. A solution of (+)-6-dimethylamino-4,4-diphenyl-3-heptanone (herein referred to as rac-methadone) hydrochloride (0.6907 g, 1.997 mmol) in water (50 mL) was added to the diclofenate solution. A white precipitate formed and the solution was stirred for 15 minutes. After an attempt to remove the precipitate by filtration was unsuccessful, the aqueous solution and precipitate were transferred to a separatory funnel using a small portion of diethyl ether to aid in the transfer. Additional diethyl ether was added to the separatory funnel (250 mL) and any remaining precipitate was dissolved with shaking. After separation of the organic and aqueous layers, the aqueous solution was washed with additional diethyl ether (2×250 mL) to extract any remaining product. The organic layers were combined and the solvent removed by rotary evaporation and the product placed under reduced pressure overnight. The resulting white solid was characterized by elemental analysis: Expected: 69.42% C, 6.33% H, 4.63% N; Obtained: 68.78% C, 6.36% H, 4.55% N; DSC: T_{g}: 31.4° C.; NMR, FTIR, and FT-Raman. Representative bands observed in the FTIR and FT-Raman spectra are listed in the Tables 7a and 7b, respectively. Resonances for the ¹and ¹³C NMR spectra are listed in Tables 8a and 8b, respectively.

TABLE 7a
Observed bands for FTIR spectrum from Example 9.

| Band (cm⁻¹) | Intensity |
|---|---|
| 3061 | weak |
| 3028 | weak |
| 2969 | weak |
| 2936 | weak |
| 1706 | moderate |
| 1587 | moderate |
| 1577 | moderate |
| 1560 | weak |
| 1497 | moderate |
| 1451 | very strong |
| 1374 | weak |
| 1305 | weak |
| 1195 | weak |
| 1150 | weak |
| 1095 | weak |
| 1046 | weak |
| 934 | weak |
| 867 | weak |
| 765 | strong |
| 747 | strong |
| 703 | very strong |

TABLE 7b
Observed bands for FT-Raman spectrum from Example 9.

| Band (cm⁻¹) | Intensity |
|---|---|
| 3064 | very strong |
| 2969 | moderate |
| 2936 | moderate |
| 1705 | weak |
| 1601 | strong |
| 1577 | strong |
| 1451 | weak |
| 1274 | weak |
| 1248 | weak |
| 1236 | moderate |
| 1191 | moderate |
| 1159 | moderate |
| 1093 | weak |
| 1071 | weak |
| 1045 | moderate |
| 1035 | moderate |
| 1002 | strong |
| 838 | weak |
| 766 | weak |
| 718 | weak |
| 619 | weak |
| 605 | weak |
| 547 | weak |
| 532 | weak |
| 517 | weak |
| 444 | weak |
| 404 | weak |
| 366 | weak |
| 317 | weak |
| 289 | weak |
| 238 | weak |
| 215 | weak |

TABLE 8a
Observed resonances for the ¹H NMR spectrum from Example 9 obtained
from d₆-DMSO solution.

| Resonance (ppm) | Multiplicity* | Number of Protons |
|---|---|---|
| 7.88 | bs | 1 |
| 7.51 | d | 2 |
| 7.24-7.36 | m | 10 |
| 7.14-7.18 | d, t | 2 |
| 7.03 | t | 1 |
| 6.83 | t | 1 |
| 6.28 | dd | 1 |
| 3.64 | s | 2 |
| 2.91 | dd | 1 |
| 2.41 | q | 1 |
| 2.30 | m | 2 |
| 2.18 | s | 6 |
| 2.03 | dd | 1 |
| 0.73 | t | 3 |
| 0.46 | d | 3 |

*s—singlet, d—doublet, dd—doublet of doublets, m—multiplet, b—broad, t—triplet, q—quartet.

TABLE 8b
Observed resonances for the ¹³C NMR spectrum from Example 9 obtained
from d₆-DMSO solution.
Resonance (ppm)

209.34
173.93
142.75
142.52
141.98
137.32
130.68
129.66
129.60
129.12
128.84
128.20
127.89
127.05
126.91
126.58
125.10
125.02
120.57
115.89
64.32
55.48
41.42
31.53
12.28
9.25

### EXAMPLE 10

### Purity Determination of Propoxyphene Diclofenate by Supercritical Fluid Chromatography

The purity of the propoxyphene diclofenate prepared in the foregoing examples was determined by utilizing supercritical fluid chromatography (“SFC”) according to the following procedure.

SFC was performed with the Analytical SFC system (Berger Instrumets, Newark, Del.), utilizing a 4.6×250 mm cyano column (Berger instruments) maintained at 40° C. The mobile phase contained a 90:10 mixture of carbon dioxide: 2.5 mM ammonium acetate in methanol. The column outlet pressure was held at 120 bar at a flow rate of 3.0 mL/minute. Standards and sample solution were prepared in methanol at about 0.5 mg/mL. Injection volume for sample and standard preparations (USP diclofenac sodium; USP propoxyphene HCl) was 10 μL and run time was less than 10 minutes. UV detection was performed at 208 nm. The chromatographic data peak areas were collected and analyzed using Millenium³² chromatography software (Waters Corporation, Milford, Mass.) to generate the % w/w assay values for the samples.

### EXAMPLE 11

### Preparation of 5R,6S,9R,13S,14R Codeine Diclofenate from Sodium Diclofenac and 5R,6S,9R,13S,14R-Codeine Sulfate

Aqueous solutions of sodium diclofenac (1.2729 g, 4.001 mmol in 100 mL of water) and 7,8-didehydro-4,5R-epoxy-3-methoxy-9R,13S,14R,17-methylmorphinan-6S-ol (herein referred to as codeine) sulfate (1.4077 g, 2.020 mmol in 60 mL of water) were combined into a 250 mL round bottom flask. A white precipitate formed and the solution was stirred for 30 minutes. The contents of the 250 mL round bottom flask were transferred to a separatory funnel using a small portion of diethyl ether to aid in the transfer. Diethyl ether (90 mL) and chloroform (90 mL) were added to the separatory funnel and any remaining precipitate was dissolved with shaking. The organic layer was separated and the solvent removed by rotary evaporation. The resulting white solid was dissolved in diethyl ether (100 mL) and the solvent again removed by rotary evaporation. The resulting white solid was dried under reduced pressure overnight (30° C.). The product was characterized by means of DSC: T_{g} at 43.2° C. degradation at 182° C.; and spectroscopically by NMR, FTIR, and FT-Raman. Representative bands observed in the FTIR and FT-Raman spectra are listed in Tables 9a and 9b, respectively. Resonances for the ¹H and ¹³C NMR spectra are listed in Tables 10a and 10b, respectively.

TABLE 9a
Observed bands for FTIR spectrum from Example 11.

| Band (cm⁻¹) | Intensity |
|---|---|
| 2936 | weak |
| 2841 | weak |
| 1634 | weak |
| 1605 | weak |
| 1587 | moderate |
| 1577 | moderate |
| 1560 | weak |
| 1504 | strong |
| 1452 | very strong |
| 1369 | weak |
| 1303 | weak |
| 1285 | moderate |
| 1274 | moderate |
| 1192 | weak |
| 1180 | weak |
| 1159 | weak |
| 1121 | weak |
| 1090 | weak |
| 1071 | weak |
| 1050 | weak |
| 1024 | weak |
| 973 | weak |
| 942 | weak |
| 930 | weak |
| 919 | weak |
| 872 | weak |
| 836 | weak |
| 749 | very strong |
| 717 | moderate |
| 696 | weak |
| 666 | weak |

TABLE 9b
Observed bands for FT-Raman spectrum from Example 11.

| Band (cm⁻¹) | Intensity |
|---|---|
| 3069 | strong |
| 2986 | very strong |
| 2947 | strong |
| 2837 | weak |
| 1635 | weak |
| 1603 | strong |
| 1578 | strong |
| 1451 | weak |
| 1280 | weak |
| 1235 | weak |
| 1195 | weak |
| 1160 | weak |
| 1092 | weak |
| 1072 | weak |
| 1046 | moderate |
| 837 | weak |
| 715 | weak |
| 667 | weak |
| 628 | weak |
| 532 | weak |
| 443 | weak |
| 366 | weak |
| 312 | weak |
| 263 | weak |

TABLE 10a
Observed resonances for the ¹H NMR spectrum from Example 11 obtained
from d₆-DMSO solution.

| Resonance (ppm) | Multiplicity* | Number of Protons |
|---|---|---|
| 7.62 | bs | 1 |
| 7.51 | d | 2 |
| 7.15-7.20 | d, t | 2 |
| 7.05 | t | 1 |
| 6.85 | t | 1 |
| 6.63 | d | 1 |
| 4.48 | d | 1 |
| 6.28 | dd | 1 |
| 5.55 | m | 1 |
| 5.25 | m | 1 |
| 4.69 | dd | 1 |
| 4.11 | m | 1 |
| 3.72 | s | 3 |
| 3.67 | s | 2 |
| 3.41 | m | 1 |
| 2.97 | d | 1 |
| 2.57-2.65 | t, dd | 2 |
| 2.40 | s | 3 |
| 2.30-2.36 | m | 2 |
| 2.03 | m | 1 |
| 1.66 | dd | 1 |

*s—singlet, d—doublet, dd—doublet of doublets, m—multiplet, b—broad, t—triplet, q—quartet.

TABLE 10b
Observed resonances for the ¹³C NMR spectrum from Example 11
obtained from d₆-DMSO solution.
Resonance (ppm)

173.80
147.20
142.71
141.35
137.23
133.62
130.76
129.79
129.15
127.78
127.24
126.73
125.28
124.59
120.67
118.47
115.92
113.37
91.77
79.16
66.35
58.04
56.01
45.76
42.65
42.17
38.74
34.73
20.48

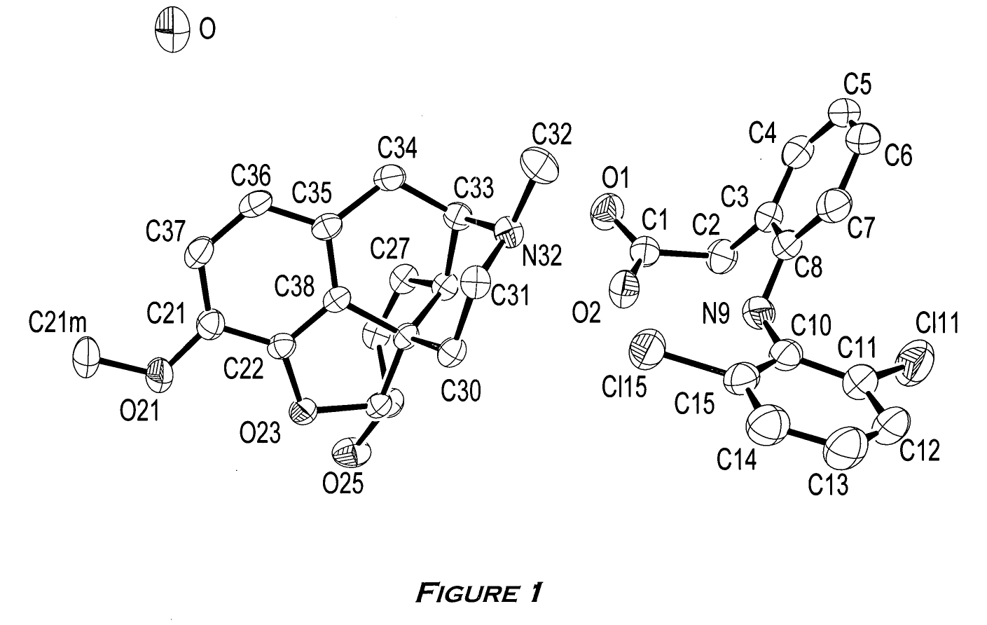

An attempt to recrystallize the product was performed. A portion of the solid (9 mg) was dissolved in a test tube with several drops of acetone. Water was added until a precipitate formed and the contents of the test tube transferred to a separatory funnel containing diethyl ether (8 mL). The solid product was dissolved in the diethyl ether, and then was extracted and the organic layer separated and set aside to evaporate. Upon evaporation of the diethyl ether, the resulting product was characterized by single crystal X-ray crystallography as codeine diclofenate monohydrate. An ORTEP representation of the product is shown in FIG. 1. Pertinent bond distances and angles corresponding to this structure are listed below in Tables 11a and 11b, respectively. Representative bands observed in the FTIR spectrum of the crystalline product are listed in Table 11c.

TABLE 11a
Selected Bond Distances from Example 11 crystalline product.

| Bond | Distance (Å) |
|---|---|
| Cl11—C11 | 1.722(3) |
| O1—C1 | 1.243(3) |
| N9—C10 | 1.388(3) |
| C1—C2 | 1.538(3) |
| C3—C4 | 1.390(3) |
| C4—C5 | 1.394(5) |
| C6—C7 | 1.374(4) |
| C10—C15 | 1.389(4) |
| C11—C12 | 1.381(4) |
| C13—C14 | 1.384(4) |
| O21—C21 | 1.367(3) |
| O23—C22 | 1.368(2) |
| O25—C25 | 1.416(3) |
| N32—C31 | 1.490(3) |
| C21—C37 | 1.385(3) |
| C22—C38 | 1.371(3) |
| C24—C29 | 1.542(3) |
| C26—C27 | 1.323(3) |
| C28—C29 | 1.539(2) |
| C29—C38 | 1.509(2) |
| C30—C31 | 1.512(3) |
| C34—C35 | 1.511(3) |
| C35—C36 | 1.398(3) |
| Cl15—C15 | 1.746(2) |
| O2—C1 | 1.260(3) |
| N9—C8 | 1.408(3) |
| C2—C3 | 1.509(4) |
| C3—C8 | 1.403(4) |
| C5—C6 | 1.372(6) |
| C7—C8 | 1.393(4) |
| C10—C11 | 1.411(3) |
| C12—C13 | 1.375(5) |
| C14—C15 | 1.376(4) |
| O21—C21M | 1.414(3) |
| O23—C24 | 1.471(2) |
| N32—C32 | 1.486(3) |
| N32—C33 | 1.513(2) |
| C21—C22 | 1.393(2) |
| C24—C25 | 1.531(3) |
| C25—C26 | 1.501(3) |
| C27—C28 | 1.513(3) |
| C28—C33 | 1.543(3) |
| C29—C30 | 1.536(2) |
| C33—C34 | 1.543(3) |
| C35—C38 | 1.383(2) |
| C36—C37 | 1.388(3) |

TABLE 11b
Selected Bond Angles from Example 11 crystalline product.

| Bond Angle | Degree |
|---|---|
| C10—N9—C8 | 122.7(2) |
| O1—C1—C2 | 119.4(2) |
| C3—C2—C1 | 111.5(2) |
| C4—C3—C2 | 121.3(3) |
| C3—C4—C5 | 121.7(3) |
| C5—C6—C7 | 119.7(3) |
| C7—C8—C3 | 119.6(2) |
| C3—C8—N9 | 118.8(2) |
| N9—C10—C11 | 121.7(3) |
| C12—C11—C10 | 121.9(3) |
| C10—C11—Cl11 | 119.4(2) |
| C15—C14—C13 | 118.9(3) |
| C14—C15—Cl15 | 118.1(2) |
| C21—O21—C21M | 117.4(2) |
| C32—N32—C31 | 110.9(2) |
| C31—N32—C33 | 111.8(2) |
| O21—C21—C22 | 116.5(2) |
| O23—C22—C38 | 113.2(1) |
| C38—C22—C21 | 120.9(2) |
| O23—C24—C29 | 107.2(1) |
| O25—C25—C26 | 110.4(2) |
| C26—C25—C24 | 111.5(2) |
| C26—C27—C28 | 118.5(2) |
| C27—C28—C33 | 114.0(2) |
| C38—C29—C30 | 112.8(2) |
| C30—C29—C28 | 109.8(1) |
| C30—C29—C24 | 111.8(1) |
| C31—C30—C29 | 111.7(2) |
| N32—C33—C28 | 106.0(1) |
| C28—C33—C34 | 114.3(2) |
| C38—C35—C36 | 116.0(12) |
| C36—C35—C34 | 125.0(2) |
| C21—C37—C36 | 121.9(2) |
| C22—C38—C29 | 109.8(1) |
| O1—C1—O2 | 124.1(2) |
| O2—C1—C2 | 116.5(2) |
| C4—C3—C8 | 117.9(3) |
| C8—C3—C2 | 120.7(2) |
| C6—C5—C4 | 119.6(3) |
| C6—C7—C8 | 121.5(3) |
| C7—C8—N9 | 121.6(3) |
| N9—C10—C15 | 122.8(2) |
| C15—C10—C11 | 115.4(2) |
| C12—C11—Cl11 | 118.8(2) |
| C13—C12—C11 | 120.1(3) |
| C14—C15—C10 | 123.7(2) |
| C10—C15—Cl15 | 118.2(2) |
| C22—O23—C24 | 107.0(1) |
| C32—N32—C33 | 113.8(2) |
| O21—C21—C37 | 126.7(2) |
| C37—C21—C22 | 116.7(2) |
| O23—C22—C21 | 125.7(2) |
| O23—C24—C25 | 110.7(2) |
| C25—C24—C29 | 111.7(2) |
| O25—C25—C24 | 113.2(2) |
| C27—C26—C25 | 119.6(2) |
| C27—C28—C29 | 108.7(1) |
| C29—C28—C33 | 107.7(2) |
| C38—C29—C28 | 105.4(1) |
| C38—C29—C24 | 100.5(1) |
| C28—C29—C24 | 116.1(2) |
| N32—C31—C30 | 111.1(2) |
| N32—C33—C34 | 112.4(2) |
| C35—C34—C33 | 114.9(2) |
| C38—C35—C34 | 118.5(2) |
| C37—C36—C35 | 121.1(2) |
| C22—C38—C35 | 122.8(2) |
| C35—C38—C29 | 126.6(2) |

TABLE 11c
Observed bands for FTIR spectrum from
Example 11 crystalline product.

| Band (cm⁻¹) | Intensity |
|---|---|
| 3282 | moderate, broad |
| 3221 | moderate, broad |
| 3072 | moderate |
| 3038 | moderate |
| 3013 | moderate |
| 2969 | moderate |
| 2945 | moderate |
| 2923 | moderate |
| 2834 | weak |
| 2778 | weak |
| 1631 | moderate |
| 1602 | moderate |
| 1586 | moderate |
| 1575 | moderate |
| 1560 | moderate |
| 1501 | strong |
| 1466 | moderate |
| 1448 | strong |
| 1369 | moderate |
| 1307 | strong |
| 1272 | strong |
| 1212 | moderate |
| 1190 | moderate |
| 1176 | moderate |
| 1161 | moderate |
| 1151 | moderate |
| 1122 | strong |
| 1097 | strong |
| 1044 | strong |
| 1019 | moderate |
| 982 | weak |
| 968 | moderate |
| 943 | moderate |
| 931 | moderate |
| 917 | weak |
| 883 | weak |
| 866 | moderate |
| 840 | moderate |
| 791 | moderate |
| 771 | strong |
| 753 | very strong |
| 717 | weak |
| 696 | weak |
| 676 | weak |

### EXAMPLE 12

### Preparation of Propoxyphene Salicylate from Propoxyphene Hydrochloride and sodium Salicylate

d-Propoxyphene hydrochloride (1.5020 g, 4.00 mmol) in water (50 mL) was placed in a 250 mL breaker. Sodium salicylate (0.6399 g, 4.00 mmol) in water (50 mL) was added to the propoxyphene solution forming a white precipitate. After mixing for 2 hours, the contents of the beaker were transferred to a separatory funnel with the aid of a small portion of diethyl ether. Additional diethyl ether was added to the separatory funnel (100 mL) and any remaining precipitate dissolved with shaking. The aqueous and organic layers were separated and the aqueous layer was washed with an additional portion of diethyl ether (100 mL) to extract any remaining product. The organic and aqueous layers were separated again, the organic layers combined, washed with water (50 mL), and the solvent removed by rotary evaporation. The resulting oily material was placed under reduced pressure to form a white solid. After drying to constant weight, the white solid was characterized by elemental analysis: Expected: 72.93% C, 7.39% H, 2.98% N; Obtained: 72.32% C, 7.38% H, 2.94% N; NMR, and FTIR. Representative bands are listed in Table 12a. Resonances for the ¹H and ¹³C NMR are listed in Tables 12b and 12c, respectively.

TABLE 12a
Observed bands for FTIR spectrum from Example 12.

| Band (cm⁻¹) | Intensity |
|---|---|
| 2974 | weak |
| 1736 | moderate |
| 1631 | moderate |
| 1594 | moderate |
| 1486 | strong |
| 1456 | strong |
| 1380 | strong |
| 1346 | moderate |
| 1323 | moderate |
| 1304 | moderate |
| 1259 | moderate |
| 1175 | strong |
| 1140 | moderate |
| 1081 | moderate |
| 1027 | moderate |
| 971 | weak |
| 916 | weak |
| 891 | weak |
| 858 | moderate |
| 807 | moderate |
| 761 | strong |
| 726 | moderate |
| 704 | very strong |
| 666 | moderate |

TABLE 12b
Observed resonances for the ¹H NMR spectrum from Example 12 obtained
from d₆-DMSO solution.

| Resonance (ppm) | Multiplicity* | Number of Protons |
|---|---|---|
| 7.70 | dd | 1 |
| 7.69-7.71 | s, d | 3 |
| 7.32-7.40 | m | 1 |
| 7.20-7.25 | m | 5 |
| 7.02-7.04 | s, d | 2 |
| 6.66-6.72 | m | 2 |
| 3.82 | q | 2 |
| 3.23 | d | 1 |
| 2.70 | m | 1 |
| 2.57 | s | 6 |
| 2.25-2.40 | m | 3 |
| 1.05 | d | 3 |
| 0.97 | t | 3 |

*s - singlet, d - doublet, dd - doublet of doublets, m - multiplet, b - broad, t - triplet, q - quartet.

TABLE 12c
Observed resonances for the ¹³C NMR spectrum from Example 12
obtained from d₆-DMSO solution.
Resonance (ppm)

172.69
172.16
162.17
138.91
136.02
132.28
130.02
128.00
127.77
127.14
126.61
126.20
118.74
116.75
116.00
86.64
59.36
43.36
38.05
35.42
28.05
14.22
8.82

### EXAMPLE 13

### Preparation of d-Propoxyphene Ibuprofenate from d-Propoxyphene Hydrochloride and rac-Ibuprofen

A solution of (±)-2-(4-Isobutylphenyl)propionic acid (herein referred to as rac-ibuprofen) (0.3071 g, 1.49 mmol) in ethanol (20 mL) was placed in a 50 mL breaker. Potassium hydroxide (0.083512 g, 1.49 mmol) in ethanol (5 mL) was added to the ibuprofen solution and stirred for 1 hour. The solvent was removed by rotary evaporation and the product dissolved in water (100 mL). Propoxyphene hydrochloride (0.5611 g, 1.49 mmol) in water (100 mL) was placed in a 500 mL beaker. The aqueous ibuprofen solution was added to the propoxyphene solution forming a white precipitate. After mixing for 1.5 hours, the contents of the 500 mL beaker were transferred to a separatory funnel with the aid of a small portion of diethyl ether. Additional diethyl ether was added to the separatory funnel (125 mL) and any remaining precipitate was dissolved with shaking. The aqueous and organic layers were separated and the aqueous layer washed with additional portions of diethyl ether (2×125 mL) to extract any remaining product. The organic layers were combined, washed with water (2×100 mL), and the solvent removed by rotary evaporation. The resulting oily material was placed under reduced pressure. After drying to constant weight, the oily material was characterized by elemental analysis: Expected: 77.03% C, 8.68% H, 2.57 % N, Obtained: 77.11% C, 8.63% H, 2.55% N; NMR, and FTIR. Representative bands are listed in Table 13a. Resonances for the ¹H and ¹³C NMR spectra are listed in Tables 13b and 13c, respectively.

TABLE 13a
Observed bands for FTIR spectrum from Example 13.

| Band (cm⁻¹) | Intensity |
|---|---|
| 2955 | moderate, broad |
| 2872 | weak |
| 1731 | strong |
| 1601 | weak |
| 1499 | weak |
| 1459 | moderate |
| 1384 | weak |
| 1223 | strong |
| 1083 | weak |
| 1079 | weak |
| 1025 | moderate |
| 964 | moderate, broad |
| 707 | weak |

TABLE 13b
Observed resonances for the ¹H NMR spectrum from Example 13 obtained
from d₆-DMSO solution.

| Resonance (ppm) | Multiplicity* | Number of Protons |
|---|---|---|
| 7.34-7.37 | m | 4 |
| 7.27-7.29 | m | 1 |
| 7.16-7.23 | m | 5 |
| 7.09 | d | 2 |
| 7.02 | dd | 2 |
| 3.81 | q | 2 |
| 3.62 | q | 1 |
| 2.63 | m | 1 |
| 2.41 | d | 2 |
| 2.23-2.28 | m | 3 |
| 1.98 | s | 6 |
| 1.80 | m | 1 |
| 1.49 | t | 1 |
| 1.35 | d | 3 |
| 0.92-0.99 | t, d | 6 |
| 0.85 | d | 6 |

*s - singlet, d - doublet, dd - doublet of doublets, m - multiplet, b - broad, t - triplet, q - quartet.

TABLE 13c
Observed resonances for the ¹³C NMR spectrum from Example 13
obtained from d₆-DMSO solution.
Resonance (ppm)

175.48
172.57
139.47
138.54
136.65
129.96
128.90
127.79
127.43
127.07
126.78
126.44
126.32
87.79
61.04
45.72
44.33
44.21
38.70
36.63
29.59
28.37
22.16
18.53
14.35
9.04

### EXAMPLE 14

### Preparation of 5R,6S,9R,13S,14R-Morphine Diclofenate from 5R,6S,9R,13S,14R-Morphine Sulfate Pentahydrate and Sodium Diclofenac

An aqueous solution (20 mL) of 7,8-didehydro-4,5R-epoxy-9R,13S,14R,17-methylmorphinan-3,6S-diol (referred to herein as morphine) sulfate pentahydrate (0.1508 g, 0.199 mmol) in water (20 mL) was placed in a 100 mL round bottom flask. Sodium diclofenac (0.1227 g, 0.386 mmol) in water (20 mL) was added to the morphine solution forming a white precipitate. After mixing for 1 hour, the precipitate was removed by filtration through a 0.45 μm polyvinylidene fluoride (PVDF) filter. The filtrate was transferred to a 50 mL round bottom flask with a small amount of acetonitrile and the solvent removed by rotary evaporation. The precipitate was dried under reduced pressure at 44° C. The white solid was characterized by FTIR with representative bands listed in Table 14a. Resonances for the ¹H and ¹³C NMR spectra are listed in Tables 14b and 14c, respectively

TABLE 14a
Observed bands for FTIR spectrum from Example 14.

| Band (cm⁻¹) | Intensity |
|---|---|
| 3034 | weak |
| 2936 | weak |
| 1636 | weak |
| 1603 | moderate |
| 1577 | moderate |
| 1557 | moderate |
| 1503 | strong |
| 1453 | very strong |
| 1373 | moderate |
| 1313 | moderate |
| 1277 | moderate |
| 1248 | moderate |
| 1192 | weak |
| 1176 | moderate |
| 1159 | moderate |
| 1123 | moderate |
| 1070 | weak |
| 1021 | weak |
| 961 | weak |
| 944 | weak |
| 871 | weak |
| 836 | weak |
| 782 | strong |
| 747 | strong |
| 714 | moderate |

TABLE 14b
Observed resonances for the ¹H NMR spectrum from Example 14 obtained
from d₆-DMSO solution.

| Resonance (ppm) | Multiplicity* | Number of Protons |
|---|---|---|
| 7.68 | bs | 1 |
| 7.51 | d | 2 |
| 7.15-7.20 | d, t | 2 |
| 7.05 | t | 1 |
| 6.85 | t | 1 |
| 6.46 | d | 1 |
| 6.36 | d | 1 |
| 6.29 | dd | 1 |
| 5.55 | d | 1 |
| 5.24 | m | 1 |
| 4.67 | dd | 1 |
| 4.09 | m | 1 |
| 3.66 | s | 2 |
| 3.40 | m | 1 |
| 2.93 | d | 1 |
| 2.58-2.63 | d, dd | 2 |
| 2.40 | s | 3 |
| 2.27-2.39 | dd, dd | 2 |
| 1.99-2.06 | m | 1 |
| 1.65 | dd | 1 |

*s - singlet, d - doublet, dd - doublet of doublets, m - multiplet, b - broad, t - triplet, q - quartet.

TABLE 14c
Observed resonances for the ¹³C NMR spectrum from Example 14
obtained from d₆-DMSO solution.
Resonance (ppm)

173.83
146.20
142.71
138.56
137.24
133.63
130.74
130.53
129.76
129.14
127.76
127.19
125.24
124.74
124.68
120.64
118.56
116.41
115.91
91.21
66.23
58.14
45.83
42.62
42.14
38.87
34.69
20.45

### EXAMPLE 15

### Preparation of 5R,9R,13R,14S-Oxycodone Diclofenate from 5R,9R,13R,14S-Oxycodone Hydrochloride and Sodium Diclofenac

An aqueous solution (20 mL) of 4,5R-epoxy-14S-hydroxy-3-methoxy-9R,13R,17-methylmorphinan-6-one (herein referred to as oxycodone) hydrochloride (0.3433 g, 0.976 mmol) in water (20 mL) was placed in a 100 mL round bottom flask. Sodium diclofenac (0.3125 g, 0.982 mmol) in water (20 mL) was added to the oxycodone solution forming a white precipitate. After mixing for 1 hour, the aqueous solution and precipitate were transferred to a separatory funnel and diethyl ether was added (20 mL). Diethyl ether (20 mL) was also added to the 100 mL round bottom flask to dissolve any remaining precipitate. This solution was added to the separatory funnel, and any precipitate in the separatory funnel was dissolved with shaking. the organic layer separated and the solvent removed by rotary evaporation. The resulting oily material was placed under reduced pressure to form a white solid. The white solid was characterized by elemental analysis: Expected: 62.85% C, 5.27% H, 4.58% N; Obtained: 62.44% C, 5.37% H, 4.41% N; NMR, and FTIR. The representative bands listed in Table 15a. Resonances for the ¹H and ¹³C NMR spectra are listed in Tables 15b and 15c, respectively.

TABLE 15a
Observed bands for FTIR spectrum from Example 15.

| Band (cm⁻¹) | Intensity |
|---|---|
| 2932 | weak |
| 2836 | weak |
| 1727 | moderate |
| 1605 | weak |
| 1578 | moderate |
| 1505 | strong |
| 1451 | very strong |
| 1383 | moderate |
| 1279 | strong |
| 1231 | weak |
| 1163 | moderate |
| 1148 | moderate |
| 1112 | weak |
| 1071 | weak |
| 1036 | moderate |
| 986 | weak |
| 944 | moderate |
| 906 | weak |
| 881 | weak |
| 848 | weak |
| 772 | strong |
| 746 | strong |
| 713 | weak |

TABLE 15b
Observed resonances for the ¹H NMR spectrum from Example 15 obtained
from d₆-DMSO solution.

| Resonance (ppm) | Multiplicity* | Number of Protons |
|---|---|---|
| 7.54 | bs | 1 |
| 7.51 | d | 2 |
| 7.15-7.21 | d, t | 2 |
| 7.05 | t | 1 |
| 6.85 | t | 1 |
| 6.76 | d | 1 |
| 6.68 | d | 1 |
| 6.29 | dd | 1 |
| 4.84 | s | 1 |
| 3.79 | s | 3 |
| 3.68 | s | 2 |
| 3.14 | d | 1 |
| 2.87-2.97 | d, m | 2 |
| 2.56 | dd | 1 |
| 2.37-2.45 | dd, s | 4 |
| 2.01-2.11 | m, dd | 2 |
| 1.75-1.80 | m | 1 |
| 1.45 | m | 1 |
| 1.31 | dd | 1 |

*s - singlet, d - doublet, dd - doublet of doublets, m - multiplet, b - broad, t - triplet, q - quartet.

TABLE 15c
Observed resonances for the ¹³C NMR spectrum from Example 15
obtained from d₆-DMSO solution.
Resonance (ppm)

208.35
173.60
144.41
142.68
142.03
137.19
130.79
129.86
129.42
129.14
127.28
125.34
125.30
124.39
120.66
119.33
115.90
114.79
89.71
69.82
63.80
56.32
49.42
45.14
38.45
35.58
31.14
29.52
21.66

### EXAMPLE 16

### Preparation of d-Propoxyphene Acetylsalicylate from d-Propoxyphene Hydrochloride and Acetylsalicylic Acid

Acetylsalicylic acid (0.5459 g, 3.03 mmol) in ethanol (60 mL) was placed in a 100 mL beaker. Potassium hydroxide (0.1694 g, 3.02 mmol) in ethanol (40 mL) was added to the acetylsalicylic acid solution and stirred for 1 hour. d-Propoxyphene hydrochloride (1.1278 g, 3.00 mmol) in water (80 mL) was placed in a 250 mL beaker. The ethanolic acetylsalicylate solution was added to the propoxyphene solution. The solution was transferred to a 500 mL round bottom flask and the volume reduced to 60 mL by rotary evaporation. After reduction, a white precipitate was observed. The contents of the 500 mL round bottom flask were transferred to a separatory funnel with the aid of a small amount of diethyl ether. Additional diethyl ether (90 mL) was added to the separatory funnel and any remaining precipitate was dissolved with shaking. The aqueous and organic layers were separated and the aqueous layer was washed with additional diethyl ether (3×90 mL) to extract any remaining product. The organic layers were combined and the solvent removed by rotary evaporation forming a viscous liquid. The viscous liquid was characterized by elemental analysis: Expected: 70.94% C, 7.94% H, 2.51% N; Obtained: 70.22% C, 7.16% H, 2.44% N (corrected for residual solvent content); TGA: 5.4% weight loss up to 160° C. and DSC: degradation >170° C.

### EXAMPLE 17

### Preparation of d-Propoxyphene Indomethacinate from d-Propoxyphene Hydrochloride and Indomethacin

d-Propoxyphene Indomethacinate may be prepared using the following synthetic scheme. A solution is prepared in a minimum volume of ethanol of 1-(4-chlorobenzoyl)-5-methoxy-2-methyl-1H-indole-3-acetic acid (herein referred to as indomethacin) (0.3578 g, 1.00 mmol) and combined with potassium hydroxide (0.05611 g, 1.00 mmol) dissolved in a minimal volume of ethanol and stirred for one hour. d-Propoxyphene hydrochloride (0.3759 g, 1.00 mmol) is dissolved in a minimal volume of water and combined with the ethanolic indomethacinate solution. The resulting solution is stirred and the total volume reduced to approximately 30 mL. The concentrated solution is transferred to a separatory funnel and the desired product extracted with diethyl ether (4×90 mL). The organic layers are combined and the solvent removed by rotary evaporation. The product is dried under vacuum overnight.

### EXAMPLE 18

### Preparation of d-Propoxyphene (S)-Naproxenate from d-Propoxyphene Hydrochloride and (S)-Naproxen Sodium

d-Propoxyphene naproxenate may be prepared using the following synthetic scheme. Aqueous solutions of (S)-6-methoxy-a-methyl-2-naphthaleneacetate (herein referred to as naproxen) sodium (0.2522 g, 1.00 mmol) and d-propoxyphene hydrochloride (0.3759 g, 1.00 mmol) are combined. The solution is stirred for 60 minutes. The resulting solution is extracted with diethyl ether (4×90 mL). The organic layers are combined and the solvent removed by rotary evaporation. The product is dried under vacuum overnight.

### EXAMPLE 19

### Preparation of d-Propoxyphene Etodolate from d-Propoxyphene Hydrochloride and Etodolac

d-Propoxyphene etodolate may be prepared using the following synthetic scheme. A solution of 1,8-diethyl-1,3,4,9-tetrahydropyrano[3,4-b]indole-1-acetic acid (herein referred to as etodolac) (0.2874 g, 1.00 mmol) in a minimal volume of ethanol is combined with potassium hydroxide (0.05611 g, 1.00 mmol) dissolved in a minimal volume of ethanol (20 mL) and stirred for 1 hour. d-Propoxyphene hydrochloride (0.3759 g, 1.00 mmol) is dissolved in a minimal volume of water and combined with the ethanolic etodolate solution. The resulting solution is stirred and the total volume reduced to approximately 30 mL. The concentrated solution is transferred to a separatory funnel and the desired product extracted with diethyl ether (4×90 mL). The organic layers are combined and the solvent removed by rotary evaporation. The product is dried under vacuum overnight.

### EXAMPLE 20

### Preparation of d-Propoxyphene (S)-Ketoprofenate from d-Propoxyphene Hydrochloride and (S)-Ketoprofen

d-Propoxyphene (S)-ketoprofenate may be prepared using the following synthetic scheme. A solution of (S)-2-(3-benzoylphenyl)propionic acid (herein referred to as ketoprofen) (0.2543 g, 1.00 mmol) prepared in a minimal volume of ethanol is combined with potassium hydroxide (0.05611 g, 1.00 mmol) dissolved in a minimal volume of ethanol and stirred for 1 hour. d-Propoxyphene hydrochloride (0.3759 g, 1.00 mmol) is dissolved in a minimal volume of water and combined with the ethanolic etodolate solution. The resulting solution is stirred and the total volume reduced to approximately 30 mL. The concentrated solution is transferred to a separatory funnel and the desired product extracted with diethyl ether (4×90 mL). The organic layers are combined and the solvent removed by rotary evaporation. The product is dried under vacuum overnight.

### EXAMPLE 21

### Preparation of d-Propoxyphene Sulindate from d-Propoxyphene Hydrochloride and Sulindac

d-Propoxyphene sulindate may be prepared using the following synthetic scheme. A solution of (Z)-5-Fluoro-2-methyl-1-[p-(methylsulfinyl)benzilidine]indenyl-3-acetic acid (herein referred to as sulindac) (0.3564 g, 1.00 mmol) is prepared in a minimal volume of ethanol and is combined with potassium hydroxide (0.05611 g, 1.00 mmol) dissolved in a minimal volume of ethanol and stirred for 1 hour. d-Propoxyphene hydrochloride (0.3759 g, 1.00 mmol) is dissolved in a minimal volume of water and combined with the ethanolic sulindate solution. The resulting solution is stirred and the total volume reduced to approximately 30 mL. The concentrated solution is transferred to a separatory funnel and the desired product extracted with diethyl ether (4×90 mL). The organic layers are combined and the solvent removed by rotary evaporation. The product is dried under vacuum overnight.

### EXAMPLE 22

### Preparation of d-Propoxyphene Suprofenate from d-Propoxyphene Hydrochloride and Suprofen

d-Propoxyphene suprofenate may be prepared using the following synthetic scheme. A solution of (α)-methyl-p-(2-thenoyl)phenylacetic acid (herein referred to as suprofen) (0.2543 g, 1.00 mmol) is prepared in a minimal volume of ethanol and is combined with potassium hydroxide (0.05611 g, 1.00 mmol) dissolved in a minimal volume of ethanol and stirred for 1 hour. d-Propoxyphene hydrochloride (0.3759 g, 1.00 mmol) is dissolved in a minimal volume of water and combined with the ethanolic suprofenate solution. The resulting solution is stirred and the total volume reduced to approximately 30 mL. The solution is transferred to a separatory funnel and the desired product extracted with diethyl ether (4×90 mL). The organic layers are combined and the solvent removed by rotary evaporation. The product is dried under vacuum overnight.

### EXAMPLE 23

### Preparation of d-Propoxyphene (S)-Flurbiprofenate from d-Propoxyphene Hydrochloride and (S)-Flurbiprofen

d-Propoxyphene (S)-flurbiprofenate may be prepared using the following synthetic scheme. A solution of (S)-2-Fluoro-α-methyl-4-biphenylacetic acid (herein referred to as flurbiprofen) (0.2443 g, 1.00 mmol) is prepared in a minimal volume of ethanol and is combined with potassium hydroxide (0.05611 g, 1.00 mmol) dissolved in a minimal volume of ethanol and stirred for 1 hour. d-Propoxyphene hydrochloride (0.3759 g, 1.00 mmol) is dissolved in a minimal volume of water and combined with the ethanolic flurbiprofenate solution. The solution is stirred and the total volume reduced to approximately 30 mL. The solution is transferred to a separatory funnel and the desired product extracted with diethyl ether (4×90 mL). The organic layers are combined and the solvent removed by rotary evaporation. The product is dried under vacuum overnight.

### EXAMPLE 24

### Preparation of d-Propoxyphene Tolmetinate from d-Propoxyphene Hydrochloride and Tolmetin Sodium Dihydrate

d-Propoxyphene tolmetinate may be prepared using the following synthetic scheme. Aqueous solutions of 1-methyl-5-(p-toluoyl)pyrrole-2-acetic acid (herein referred to as tolmetin) sodium dihydrate (0.3153 g, 1.00 mmol) and d-Propoxyphene hydrochloride (0.3759 g, 1.00 mmol) are combined into an suitable flask and stirred for 60 minutes. The resulting solution is transferred to a separatory funnel and the desired product extracted with diethyl ether (4×90 mL). The organic layers are combined and the solvent removed by rotary evaporation. The product is dried under vacuum overnight.

### EXAMPLE 25

### Preparation of d-Propoxyphene Fenoprofenate from d-Propoxyphene Hydrochloride and Fenoprofen Calcium Trihydrate

d-Propoxyphene fenoprofenate may be prepared using the following synthetic scheme. Aqueous solutions of (±)-2-(3-phenoxyphenyl)propionic acid (herein referred to as fenoprofen) calcium trihydrate (0.2884 g, 0.50 mmol) and d-propoxyphene hydrochloride (0.3759 g, 1.00 mmol) are combined into an suitable flask and stirred for 60 minutes. The resulting solution is transferred to a separatory funnel and the desired product extracted with diethyl ether (4×90 mL). The organic layers are combined and the solvent removed by rotary evaporation. The product is dried under vacuum overnight.

### EXAMPLE 26

### Preparation of d-Propoxyphene Oxaprozinate from d-Propoxyphene Hydrochloride and Oxaprozin

d-Propoxyphene oxaprozinate may be prepared using the following synthetic scheme. A solution of 4,5-diphenyl-2-oxazolepropionic acid (herein referred to as oxaprozin) (0.2933 g, 1.00 mmol) is prepared in a minimal volume of ethanol and is combined with potassium hydroxide (0.05611 g, 1.00 mmol) dissolved in a minimal volume of ethanol and stirred for 1 hour. d-Propoxyphene hydrochloride (0.3759 g, 1.00 mmol) is dissolved in a minimal volume of water and combined with the ethanolic oxaprozinate solution. The resulting solution is stirred and the total volume reduced to approximately 30 mL. The concentrated solution is transferred to a separatory funnel and the desired product extracted with diethyl ether (4×90 mL). The organic layers are combined and the solvent removed by rotary evaporation. The product is dried under vacuum overnight.

### EXAMPLE 27

### Preparation of d-Propoxyphene Difunisalate from d-Propoxyphene Hydrochloride and Difusinal

d-Propoxyphene difunisalate may be prepared using the following synthetic scheme. A solution of 5-(2,4-difluorophenyl)salicylic acid (herein referred to as difunisal) (0.2502 g, 1.00 mmol) is prepared in a minimal volume of ethanol and is combined with potassium hydroxide (0.05611 g, 1.00 mmol) dissolved in a minimal volume of ethanol and stirred for 1 hour. d-Propoxyphene hydrochloride (0.3759 g, 1.00 mmol) is dissolved in a minimal volume of water and combined with the ethanolic difunisalate solution. The resulting solution is stirred and the total volume reduced to approximately 30 mL. The concentrated solution is transferred to a separatory funnel and the desired product extracted with diethyl ether (4×90 mL). The organic layers are combined and the solvent removed by rotary evaporation. The product is dried under vacuum overnight.

### EXAMPLE 28

### Preparation of d-Propoxyphene Loxoprofenate from d-Propoxyphene Hydrochloride and Loxoprofen

d-Propoxyphene loxoprofenate may be prepared using the following synthetic scheme. A solution of α-methyl-{4-[(2-oxocyclopentyl)methyl]}phenylacetic acid (herein referred to as loxoprofen) (0.2463 g, 1.00 mmol) is prepared in a minimal volume of ethanol and is combined with potassium hydroxide (0.05611 g, 1.00 mmol) dissolved in a minimal volume of ethanol and stirred for 1 hour. d-Propoxyphene hydrochloride (0.3759 g, 1.00 mmol) is dissolved in a minimal volume of water and combined with the ethanolic loxoprofenate solution. The resulting solution is stirred and the total volume reduced to approximately 30 mL. The concentrated solution is transferred to a separatory funnel and the desired product extracted with diethyl ether (4×90 mL). The organic layers are combined and the solvent removed by rotary evaporation. The product is dried under vacuum overnight.

### EXAMPLE 29

### Preparation of rac-Ketamine Ibuprofenate from rac-Ketamine Hydrochloride and Ibuprofen

rac-Ketamine ibuprofenate may be prepared using the following synthetic scheme. Ibuprofen (0.2063 g, 1.00 mmol) dissolved in a minimal volume of ethanol is combined with potassium hydroxide (0.05611 g, 1.00 mmol) dissolved in a minimal volume of ethanol and stirred for 1 hour. rac-Ketamine hydrochloride (0.2742 g, 1.00 mmol) is dissolved in a minimal volume of water and combined with the ethanolic ibuprofenate solution. The resulting solution is stirred and the total volume reduced to approximately 30 mL. The concentrated solution is transferred to a separatory funnel and the desired product extracted with diethyl ether (4×90 mL). The organic layers are combined and the solvent removed by rotary evaporation. The product is dried under vacuum overnight.

### EXAMPLE 30

### Preparation of rac-Ketamine Acetylsalicylate from rac-Ketamine Hydrochloride and Acetylsalicylic Acid

rac-Ketamine acetylsalicylate may be prepared using the following synthetic scheme. Acetylsalicylic acid (0.3003 g, 1.00 mmol) dissolved in a minimal volume of ethanol is combined with potassium hydroxide (0.05611 g, 1.00 mmol) dissolved in a minimal volume of ethanol and stirred for 1 hour. rac-Ketamine hydrochloride (0.2742 g, 1.00 mmol) is dissolved in a minimal volume of water and combined with the ethanolic acetylsalicylate solution. The resulting solution is stirred and the total volume reduced to approximately 30 mL. The solution is transferred to a separatory funnel and the desired product extracted with diethyl ether (4×90 mL). The organic layers are combined and the solvent removed by rotary evaporation. The product is dried under vacuum overnight.

### EXAMPLE 31

### Preparation of rac-Ketamine Salicylate from rac-Ketamine Hydrochloride and Sodium Salicylate

rac-Ketamine salicylate may be prepared using the following synthetic scheme. Aqueous solutions of sodium salicylate (0.1601 g, 1.00 mmol) and of rac-ketamine hydrochloride (0.2742 g, 1.00 mmol) are combined into a suitable flask and stirred for 60 minutes. The resulting solution is transferred to a separatory funnel and the desired product extracted with diethyl ether (4×90 mL). The organic layers are combined and the solvent removed by rotary evaporation. The product is dried under vacuum overnight.

### EXAMPLE 32

### Preparation of rac-Ketamine Indomethacinate from rac-Ketamine Hydrochloride and Indomethacin

rac-Ketamine indomethacinate may be prepared using the following synthetic scheme. Indomethacin (0.3578 g, 1.00 mmol) dissolved in a minimal volume of ethanol is combined with potassium hydroxide (0.05611 g, 1.00 mmol) dissolved in a minimal volume of ethanol and stirred for 1 hour. rac-Ketamine hydrochloride (0.2742 g, 1.00 mmol) is dissolved in a minimal volume of water and combined with the ethanolic indomethacinate solution. The resulting solution is stirred and the total volume reduced to approximately 30 mL. The concentrated solution is transferred to a separatory funnel and the desired product extracted with diethyl ether (4×90 mL). The organic layers are combined and the solvent removed by rotary evaporation. The product is dried under vacuum overnight.

### EXAMPLE 33

### Preparation of rac-Ketamine Naproxenate from rac-Ketamine Hydrochloride and Naproxen Sodium

rac-Ketamine naproxenate may be prepared using the following synthetic scheme. Aqueous solutions of naproxen sodium (0.2522 g, 1.00 mmol) and of rac-ketamine hydrochloride (0.2742 g, 1.00 mmol) are combined into a suitable flask and stirred for approximately 60 minutes. The resulting solution is transferred to a separatory funnel and the desired product extracted with diethyl ether (4×90 mL). The organic layers are combined and the solvent removed by rotary evaporation. The product is dried under vacuum overnight.

### EXAMPLE 34

### Preparation of rac-Ketamine Etodolate from rac-Ketamine Hydrochloride and Etodolac

rac-Ketamine etodolate may be prepared using the following synthetic scheme. Etodolac (0.2874 g, 1.00 mmol) dissolved in a minimal volume of ethanol is combined with potassium hydroxide (0.05611 g, 1.00 mmol) dissolved in a minimal volume of ethanol and stirred for 1 hour. rac-Ketamine hydrochloride (0.2742 g, 1.00 mmol) is dissolved in a minimal volume of water and combined with the ethanolic etodolate solution. The resulting solution is stirred and the total volume reduced to approximately 30 mL. The concentrated solution is transferred to a separatory funnel and the desired product extracted with diethyl ether (4×90 mL). The organic layers are combined and the solvent removed by rotary evaporation. The product is dried under vacuum overnight.

### EXAMPLE 35

### Preparation of rac-Ketamine Sulindate from rac-Ketamine Hydrochloride and Sulindac

rac-Ketamine sulindate may be prepared using the following synthetic scheme. Sulindac (0.3564 g, 1.00 mmol) dissolved in a minimal volume of ethanol is combined with potassium hydroxide (0.05611 g, 1.00 mmol) dissolved in a minimal volume of ethanol and stirred for 1 hour. rac-Ketamine hydrochloride (0.2742 g, 1.00 mmol) is dissolved in a minimal volume of water and combined with the ethanolic sulindate solution. The resulting solution is stirred and the total volume reduced to approximately 30 mL. The concentrated solution is transferred to a separatory funnel and the desired product extracted with diethyl ether (4×90 mL). The organic layers are combined and the solvent removed by rotary evaporation. The product is dried under vacuum overnight.

### EXAMPLE 36

### Preparation of rac-Ketamine (S)-Ketoprofenate from rac-Ketamine Hydrochloride and (S)-Ketoprofen

rac-Ketamine (S)-ketoprofenate may be prepared using the following synthetic scheme. (S)-Ketoprofen (0.2543 g, 1.00 mmol) dissolved in a minimal volume of ethanol is combined with potassium hydroxide (0.05611 g, 1.00 mmol) dissolved in a minimal volume of ethanol and stirred for 1 hour. rac-Ketamine hydrochloride (0.2742 g, 1.00 mmol) is dissolved in a minimal volume of water and combined with the ethanolic ketoprofenate solution. The resulting solution is stirred and the total volume reduced to approximately 30 mL. The concentrated solution is transferred to a separatory funnel and the desired product extracted with diethyl ether (4×90 mL). The organic layers are combined and the solvent removed by rotary evaporation. The product is dried under vacuum overnight.

### EXAMPLE 37

### Preparation of rac-Ketamine Suprofenate from rac-Ketamine Hydrochloride and Suprofen

rac-Ketamine suprofenate may be prepared using the following synthetic scheme. Suprofen (0.2603 g, 1.00 mmol) dissolved in a minimal volume of ethanol is combined with potassium hydroxide (0.05611 g, 1.00 mmol) dissolved in a minimal volume of ethanol and stirred for 1 hour. rac-Ketamine hydrochloride (0.2742 g, 1.00 mmol) dissolved in a minimal volume of water is combined with the ethanolic suprofenate solution. The resulting solution is stirred and the total volume reduced to approximately 30 mL. The concentrated solution is transferred to a separatory funnel and the desired product extracted with diethyl ether (4×90 mL). The organic layers are combined and the solvent removed by rotary evaporation. The product is dried under vacuum overnight.

### EXAMPLE 38

### Preparation of rac-Ketamine (S)-Flurbiprofenate from rac-Ketamine Hydrochloride and (S)-Flurbiprofen

rac-Ketamine (S)-Flurbiprofenate may be prepared using the following synthetic scheme. (S)-Flurbiprofen (0.2443 g, 1.00 mmol) dissolved in a minimal volume of ethanol is combined with potassium hydroxide (0.05611 g, 1.00 mmol) dissolved in a minimal volume of ethanol and stirred for 1 hour. rac-Ketamine hydrochloride (0.2742 g, 1.00 mmol) is dissolved in a minimal volume of water and combined with the ethanolic flurbiprofenate solution. The resulting solution is stirred and the total volume reduced to approximately 30 mL. The solution is transferred to a separatory funnel and the desired product extracted with diethyl ether (4×90 mL). The organic layers are combined and the solvent removed by rotary evaporation. The product is dried under vacuum overnight.

### EXAMPLE 39

### Preparation of rac-Ketamine Tolmetinate from rac-Ketamine Hydrochloride and Tolmetin Sodium Dihydrate

rac-Ketamine tolmetinate may be prepared using the following synthetic scheme. Aqueous solutions of tolmetin sodium dihydrate (0.3153 g, 1.00 mmol) and of rac-ketamine hydrochloride (0.2742 g, 1.00 mmol) are combined into a suitable flask and stirred for 60 minutes. The resulting solution is transferred to a separatory funnel and the desired product extracted with diethyl ether (4×90 mL). The organic layers are combined and the solvent removed by rotary evaporation. The product is dried under vacuum overnight.

### EXAMPLE 40

### Preparation of rac-Ketamine Fenoprofenate from rac-Ketamine Hydrochloride and Fenoprofen Calcium Trihydrate

rac-Ketamine fenoprofenate may be prepared using the following synthetic scheme. Aqueous solutions of fenoprofen calcium trihydrate (0.2884 g, 0.50 mmol) and of rac-ketamine hydrochloride (0.2742 g, 1.00 mmol) are combined into a suitable flask and stirred for 60 minutes. The resulting solution is transferred to a separatory funnel and the desired product extracted with diethyl ether (4×90 mL). The organic layers are combined and the solvent removed by rotary evaporation. The product is dried under vacuum overnight.

### EXAMPLE 41

### Preparation of rac-Ketamine Oxaprozinate from rac-Ketamine Hydrochloride and Oxaprozin

rac-Ketamine oxaprozinate may be prepared using the following synthetic scheme. Oxaprozin (0.2933 g, 1.00 mmol) dissolved in a minimal volume of ethanol is combined with potassium hydroxide (0.05611 g, 1.00 mmol) dissolved in a minimal volume of ethanol and stirred for 1 hour. rac-Ketamine hydrochloride (0.2742 g, 1.00 mmol) is dissolved in a minimal volume of water and combined with the ethanolic oxaprozinate solution. The resulting solution is stirred and the total volume reduced to approximately 30 mL. The concentrated solution is transferred to a separatory funnel and the desired product extracted with diethyl ether (4×90 mL). The organic layers are combined and the solvent removed by rotary evaporation. The product is dried under vacuum overnight.

### EXAMPLE 42

### Preparation of rac-Ketamine Difunisalate from rac-Ketamine Hydrochloride and Difunisal

rac-Ketamine difunisalate may be prepared using the following synthetic scheme. Difunisal (0.2502 g, 1.00 mmol) dissolved in a minimal volume of ethanol is combined with potassium hydroxide (0.05611 g, 1.00 mmol) dissolved in a minimal volume of ethanol and stirred for 1 hour. rac-Ketamine hydrochloride (0.2742 g, 1.00 mmol) is dissolved in a minimal volume of water and combined with the ethanolic difunisalate solution. The resulting solution is stirred and the total volume reduced to approximately 30 mL. The concentrated solution is transferred to a separatory funnel and the desired product extracted with diethyl ether (4×90 mL). The organic layers are combined and the solvent removed by rotary evaporation. The product is dried under vacuum overnight.

### EXAMPLE 42

### Preparation of rac-Ketamine Loxoprofenate from rac-Ketamine Hydrochloride and Loxoprofen

rac-Ketamine loxoprofenate may be prepared using the following synthetic scheme. Loxoprofen (0.2463 g, 1.00 mmol) dissolved in a minimal volume of ethanol is combined with potassium hydroxide (0.05611 g, 1.00 mmol) dissolved in a minimal volume of ethanol and stirred for 1 hour. rac-Ketamine hydrochloride (0.2742 g, 1.00 mmol) is dissolved in a minimal volume of water and combined with the ethanolic loxoprofenate solution. The resulting solution is stirred and the total volume reduced to approximately 30 mL. The concentrated solution is transferred to a separatory funnel and the desired product extracted with diethyl ether (4×90 mL). The organic layers are combined and the solvent removed by rotary evaporation. The product is dried under vacuum overnight.

### EXAMPLE 43

### Preparation of (S)-Ketamine Ibuprofenate from (S)-Ketamine Hydrochloride and Ibuprofen

(S)-Ketamine ibuprofenate may be prepared using the following synthetic scheme. Ibuprofen (0.2063 g, 1.00 mmol) dissolved in minimal volume of ethanol is combined with potassium hydroxide (0.05611 g, 1.00 mmol) dissolved in a minimal volume of ethanol and stirred for 1 hour. (S)-Ketamine hydrochloride (0.2742 g, 1.00 mmol) is dissolved in a minimal volume of water and combined with the ethanolic ibuprofenate solution. The resulting solution is stirred and the total volume reduced to approximately 30 mL. The concentrated solution is transferred to a separatory funnel and the desired product extracted with diethyl ether (4×90 mL). The organic layers are combined and the solvent removed by rotary evaporation. The product is dried under vacuum overnight.

### EXAMPLE 44

### Preparation of (S)-Ketamine Acetylsalicylate from (S)-Ketamine Hydrochloride and Acetylsalicylic Acid

(S)-Ketamine acetylsalicylate may be prepared using the following synthetic scheme. Acetylsalicylic acid (0.3003 g, 1.00 mmol) dissolved in a minimal volume of ethanol is combined with potassium hydroxide (0.05611 g, 1.00 mmol) dissolved in a minimal volume of ethanol and stirred for 1 hour. (S)-Ketamine hydrochloride (0.2742 g, 1.00 mmol) is dissolved in a minimal amount of water and combined with the ethanolic acetylsalicylate solution. The resulting solution is stirred and the total volume reduced to approximately 30 mL. The concentrated solution is transferred to a separatory funnel and the desired product extracted with diethyl ether (4×90 mL). The organic layers are combined and the solvent removed by rotary evaporation. The product is dried under vacuum overnight.

### EXAMPLE 45

### Preparation of (S)-Ketamine Salicylate from (S)-Ketamine Hydrochloride and Sodium Salicylate

(S)-Ketamine Salicylate may be prepared using the following synthetic scheme. Aqueous solutions of sodium salicylate (0.1601 g, 1.00 mmol) and of (S)-ketamine hydrochloride (0.2742 g, 1.00 mmol) are combined into a suitable flask and stirred for 60 minutes. The resulting solution is transferred to a separatory funnel and the desired product extracted with diethyl ether (4×90 mL). The organic layers are combined and the solvent removed by rotary evaporation. The product is dried under vacuum overnight.

### EXAMPLE 46

### Preparation of (S)-Ketamine Indomethacinate from (S)-Ketamine Hydrochloride and Indomethacin

(S)-Ketamine indomethacinate may be prepared using the following synthetic scheme. Indomethacin (0.3578 g, 1.00 mmol) dissolved in a minimal volume of ethanol is combined with potassium hydroxide (0.05611 g, 1.00 mmol) dissolved in a minimal volume of ethanol and stirred for 1 hour. (S)-Ketamine hydrochloride (0.2742 g, 1.00 mmol) is dissolved in a minimal volume of water and combined with the ethanolic indomethacinate solution. The resulting solution is stirred and the total volume reduced to approximately 30 mL. The concentrated solution is transferred to a separatory funnel and the desired product extracted with diethyl ether (4×90 mL). The organic layers are combined and the solvent removed by rotary evaporation. The product is dried under vacuum overnight.

### EXAMPLE 47

### Preparation of (S)-Ketamine Naproxenate from (S)-Ketamine Hydrochloride and Naproxen Sodium

(S)-Ketamine Naproxenate may be prepared using the following synthetic scheme. Aqueous solutions of naproxen sodium (0.2522 g, 1.00 mmol) and of (S)-ketamine hydrochloride (0.2742 g, 1.00 mmol) are combined into a suitable flask and stirred for 60 minutes. The resulting solution is transferred to a separatory funnel and the desired product extracted with diethyl ether (4×90 mL). The organic layers are combined and the solvent removed by rotary evaporation. The product is dried under vacuum overnight.

### EXAMPLE 48

### Preparation of (S)-Ketamine Etodolate from (S)-Ketamine Hydrochloride and Etodolac

(S)-Ketamine etodolate may be prepared using the following synthetic scheme. Etodolac (0.2874 g, 1.00 mmol) dissolved in a minimal volume of ethanol is combined with potassium hydroxide (0.05611 g, 1.00 mmol) dissolved in a minimal volume of ethanol and stirred for 1 hour. (S)-Ketamine hydrochloride (0.2742 g, 1.00 mmol) is dissolved in a minimal volume of water and combined with the ethanolic etodolate solution. The resulting solution is stirred and the total volume reduced to approximately 30 mL. The concentrated solution is transferred to a separatory funnel and the desired product extracted with diethyl ether (4×90 mL). The organic layers are combined and the solvent removed by rotary evaporation. The product is dried under vacuum overnight.

### EXAMPLE 49

### Preparation of (S)-Ketamine Sulindate from (S)-Ketamine Hydrochloride and Sulindac

(S)-Ketamine sulindate may be prepared using the following synthetic scheme. Sulindac (0.3564 g, 1.00 mmol) dissolved in a minimal volume of ethanol is combined with potassium hydroxide (0.05611 g, 1.00 mmol) dissolved in a minimal volume of ethanol and stirred for 1 hour. (S)-Ketamine hydrochloride (0.2742 g, 1.00 mmol) is dissolved in a minimal volume of water and combined with the ethanolic sulindate solution. The resulting solution is stirred and the total volume reduced to approximately 30 mL. The concentrated solution is transferred to a separatory funnel and the desired product extracted with diethyl ether (4×90 mL). The organic layers are combined and the solvent removed by rotary evaporation. The product is dried under vacuum overnight.

### EXAMPLE 50

### Preparation of (S)-Ketamine (S)-Ketoprofenate from (S)-Ketamine Hydrochloride and (S)-Ketoprofen

(S)-Ketamine (S)-ketoprofenate may be prepared using the following synthetic scheme. (S)-Ketoprofen (0.2543 g, 1.00 mmol) dissolved in a minimal volume of ethanol is combined with potassium hydroxide (0.05611 g, 1.00 mmol) dissolved in a minimal volume of ethanol and stirred for 1 hour. (S)-Ketamine hydrochloride (0.2742 g, 1.00 mmol) is dissolved in a minimal volume of water and combined with the ethanolic ketoprofenate solution. The resulting solution is stirred and the total volume reduced to approximately 30 mL. The concentrated solution is transferred to a separatory funnel and the desired product extracted with diethyl ether (4×90 mL). The organic layers are combined and the solvent removed by rotary evaporation. The product is dried under vacuum overnight.

### EXAMPLE 51

### Preparation of (S)-Ketamine Suprofenate from (S)-Ketamine Hydrochloride and Suprofen

(S)-Ketamine suprofenate may be prepared using the following synthetic scheme. Suprofen (0.2603 g, 1.00 mmol) dissolved in a minimal volume of ethanol is combined with potassium hydroxide (0.05611 g, 1.00 mmol) dissolved in a minimal volume of ethanol and stirred for 1 hour. (S)-Ketamine hydrochloride (0.2742 g, 1.00 mmol) is dissolved in a minimal volume of water and combined with the ethanolic suprofenate solution. The resulting solution is stirred and total volume reduced to approximately 30 mL. The concentrated solution is transferred to a separatory funnel and the desired product extracted with diethyl ether (4×90 mL). The organic layers are combined and the solvent removed by rotary evaporation. The product is dried under vacuum overnight.

### EXAMPLE 52

### Preparation of (S)-Ketamine (S)-Flurbiprofenate from (S)-Ketamine Hydrochloride and (S)-Flurbiprofen

(S)-Ketamine (S)-flurbiprofenate may be prepared using the following synthetic scheme. (S)-Flurbiprofen (0.2443 g, 1.00 mmol) dissolved in a minimal volume of ethanol is combined with potassium hydroxide (0.05611 g, 1.00 mmol) dissolved in a minimal volume of ethanol and stirred for 1 hour. (S)-Ketamine hydrochloride (0.2742 g, 1.00 mmol) is dissolved in a minimal volume of water and combined with the ethanolic flurbiprofenate solution. The resulting solution is stirred and the total volume reduced to approximately 30 mL. The concentrated solution is transferred to a separatory funnel and the desired product extracted with diethyl ether (4×90 mL). The organic layers are combined and the solvent removed by rotary evaporation. The product is dried under vacuum overnight.

### EXAMPLE 53

### Preparation of (S)-Ketamine Tolmetinate from (S)-Ketamine Hydrochloride and Tolmetin Sodium Dihydrate

(S)-Ketamine tolmetinate may be prepared using the following synthetic scheme. Aqueous solutions of tolmetin sodium dihydrate (0.3153 g, 1.00 mmol) and of (S)-ketamine hydrochloride (0.2742 g, 1.00 mmol) are combined into a suitable flask and stirred for 60 minutes. The resulting solution is transferred to a separatory funnel and the desired product extracted with diethyl ether (4×90 mL). The organic layers are combined and the solvent removed by rotary evaporation. The product is dried under vacuum overnight.

### EXAMPLE 54

### Preparation of (S)-Ketamine Fenoprofenate from (S)-Ketamine Hydrochloride and Fenoprofen Calcium Trihydrate

(S)-Ketamine Fenoprofenate may be prepared using the following synthetic scheme. Aqueous solutions of fenoprofen calcium trihydrate (0.2884 g, 0.50 mmol) and of (S)-ketamine hydrochloride (0.2742 g, 1.00 mmol) are combined into a suitable flask and stirred for 60 minutes. The resulting solution is transferred to a separatory funnel and the desired product extracted with diethyl ether (4×90 mL). The organic layers are combined and the solvent removed by rotary evaporation. The product is dried under vacuum overnight.

### EXAMPLE 55

### Preparation of (S)-Ketamine Oxaprozinate from (S)-Ketamine Hydrochloride and Oxaprozin

(S)-Ketamine oxaprozinate may be prepared using the following synthetic scheme. Oxaprozin (0.2933 g, 1.00 mmol) dissolved in a minimal volume of ethanol is combined with potassium hydroxide (0.05611 g, 1.00 mmol) dissolved in a minimal volume of ethanol and stirred for 1 hour. (S)-Ketamine hydrochloride (0.2742 g, 1.00 mmol) is dissolved in a minimal volume of water and combined with the ethanolic oxaprozinate solution. The resulting solution is stirred and the total volume reduced to approximately 30 mL. The concentrated solution is transferred to a separatory funnel and the desired product extracted with diethyl ether (4×90 mL). The organic layers are combined and the solvent removed by rotary evaporation. The product is dried under vacuum overnight.

### EXAMPLE 56

### Preparation of (S)-Ketamine Difunisalate from (S)-Ketamine Hydrochloride and Difunisal

(S)-Ketamine difunisalate may be prepared using the following synthetic scheme. Difunisal (0.2502 g, 1.00 mmol) dissolved in a minimal volume of ethanol is combined with potassium hydroxide (0.05611 g, 1.00 mmol) dissolved in a minimal volume of ethanol and stirred for 1 hour. (S)-Ketamine hydrochloride (0.2742 g, 1.00 mmol) is dissolved in a minimal volume of water and combined with the ethanolic difunisalate solution. The resulting solution is stirred and the total volume reduced to approximately 30 mL. The concentrated solution is transferred to a separatory funnel and the desired product extracted with diethyl ether (4×90 mL). The organic layers are combined and the solvent removed by rotary evaporation.

### EXAMPLE 55

### Preparation of (S)-Ketamine Loxoprofenate from (S)-Ketamine Hydrochloride and Loxoprofen

(S)-Ketamine loxoprofenate may be prepared using the following synthetic scheme. Loxoprofen (0.2463 g, 1.00 mmol) dissolved in a minimal volume of ethanol is combined with potassium hydroxide (0.05611 g, 1.00 mmol) dissolved in a minimal volume of ethanol and stirred for 1 hour. (S)-Ketamine hydrochloride (0.2742 g, 1.00 mmol) is dissolved in a minimal volume of water and combined with the ethanolic loxoprofenate solution. The resulting solution is stirred and the total volume reduced to approximately 30 mL. The concentrated solution is transferred to a separatory funnel and the desired product extracted with diethyl ether (4×90 mL). The organic layers are combined and the solvent removed by rotary evaporation. The product is dried under vacuum overnight.

### EXAMPLE 56

### Preparation of (S)-Ketamine Diclofenate from of (S)-Ketamine Hydrochloride and Sodium Diclofenac

(S)-Ketamine diclofenate may be prepared using the following synthetic scheme. Aqueous solutions of sodium diclofenac (0.3181 g, 1.00 mmol) and of (S)-ketamine hydrochloride (0.2742 g, 1.00 mmol) are combined into a suitable flask and stirred for 60 minutes. The resulting solution is transferred to a separatory funnel and the desired product extracted with diethyl ether (4×90 mL). The organic layers are combined and the solvent removed by rotary evaporation. The product is dried under vacuum overnight.

### EXAMPLE 57

### Preparation of rac-Methadone Ibuprofenate from rac-Methadone Hydrochloride and Ibuprofen

rac-Methadone ibuprofenate may be prepared using the following synthetic scheme. Ibuprofen (0.2063 g, 1.00 mmol) dissolved in a minimal volume of ethanol is combined with potassium hydroxide (0.05611 g, 1.00 mmol) dissolved in a minimal volume of ethanol and stirred for 1 hour. rac-Methadone hydrochloride (0.3459 g, 1.00 mmol) is dissolved in a minimal volume of water and combined with the ethanolic ibuprofenate solution. The resulting solution is stirred and the total volume is reduced to approximately 30 mL. The concentrated solution is transferred to a separatory funnel and the desired product extracted with diethyl ether (4×90 mL). The organic layers are combined and the solvent removed by rotary evaporation. The product is dried under vacuum overnight.

### EXAMPLE 58

### Preparation of rac-Methadone Acetylsalicylate from rac-Methadone Hydrochloride and Acetylsalicylic Acid

rac-Methadone acetylsalicylate may be prepared using the following synthetic scheme. Acetylsalicylic acid (0.3003 g, 1.00 mmol) dissolved in a minimal volume of ethanol is combined with potassium hydroxide (0.05611 g, 1.00 mmol) dissolved in a minimal volume of ethanol and stirred for 1 hour. rac-Methadone hydrochloride (0.3459 g, 1.00 mmol) is dissolved in a minimal volume of water and combined with the ethanolic acetylsalicylate solution. The resulting solution is stirred and the total volume reduced to approximately 30 mL. The solution is transferred to a separatory funnel and the desired product extracted with diethyl ether (4×90 mL). The organic layers are combined and the solvent removed by rotary evaporation. The product is dried under vacuum overnight.

### EXAMPLE 59

### Preparation of rac-Methadone Salicylate from rac-Methadone Hydrochloride and Sodium Salicylate

rac-Methadone salicylate may be prepared using the following synthetic scheme. Aqueous solutions of sodium salicylate (0.1601 g, 1.00 mmol) and of rac-methadone hydrochloride (0.3459 g, 1.00 mmol) are combined into a suitable flask and stirred for 60 minutes. The resulting solution is transferred to a separatory funnel and the desired product extracted with diethyl ether (4×90 mL). The organic layers are combined and the solvent removed by rotary evaporation. The product is dried under vacuum overnight.

### EXAMPLE 60

### Preparation of rac-Methadone Indomethacinate from rac-Methadone Hydrochloride and Indomethacin

rac-Methadone indomethacinate may be prepared using the following synthetic scheme. Indomethacin (0.3578 g, 1.00 mmol) dissolved in a minimal volume of ethanol is combined with potassium hydroxide (0.05611 g, 1.00 mmol) dissolved in a minimal volume of ethanol and stirred for 1 hour. rac-Methadone hydrochloride (0.3459 g, 1.00 mmol) is dissolved in a minimal volume of water and combined with the ethanolic indomethacinate solution. The resulting solution is stirred and the total volume reduced to approximately 30 mL. The concentrated solution is transferred to a separatory funnel and the desired product extracted with diethyl ether (4×90 mL). The organic layers are combined and the solvent removed by rotary evaporation. The product is dried under vacuum overnight.

### EXAMPLE 61

### Preparation of rac-Methadone Naproxenate from rac-Methadone Hydrochloride and Naproxen Sodium

rac-Methadone naproxenate may be prepared using the following synthetic scheme. Aqueous solutions of naproxen sodium (0.2522 g, 1.00 mmol) and of rac-methadone hydrochloride (0.3459 g, 1.00 mmol) are combined into a suitable flask and stirred for 60 minutes. The resulting solution is transferred to a separatory funnel and the desired product extracted with diethyl ether (4×90 mL). The organic layers are combined and the solvent removed by rotary evaporation. The product is dried under vacuum overnight.

### EXAMPLE 62

### Preparation of rac-Methadone Etodolate from rac-Methadone Hydrochloride and Etodolac

rac-Methadone etodolate may be prepared using the following synthetic scheme. Etodolac (0.2874 g, 1.00 mmol) dissolved in a minimal volume of ethanol is combined with potassium hydroxide (0.05611 g, 1.00 mmol) dissolved in a minimal volume of ethanol and stirred for 1 hour. rac-Methadone hydrochloride (0.3459 g, 1.00 mmol) is dissolved in a minimal volume of water and combined with the ethanolic etodolate solution. The resulting solution is stirred and the total volume reduced to approximately 30 mL. The concentrated solution is transferred to a separatory funnel and the desired product extracted with diethyl ether (4×90 mL). The organic layers are combined and the solvent removed by rotary evaporation. The product is dried under vacuum overnight.

### EXAMPLE 63

### Preparation of rac-Methadone Sulindate from rac-Methadone Hydrochloride and Sulindac

rac-Methadone sulindate may be prepared using the following synthetic scheme. Sulindac (0.3564 g, 1.00 mmol) dissolved in a minimal volume of ethanol is combined with potassium hydroxide (0.05611 g, 1.00 mmol) dissolved in a minimal volume of ethanol and stirred for 1 hour. rac-Methadone hydrochloride (0.3459 g, 1.00 mmol) is dissolved in a minimal volume of water and combined with the ethanolic sulindate solution. The resulting solution is stirred and the total volume reduced to approximately 30 mL. The concentrated solution is transferred to a separatory funnel and the desired product extracted with diethyl ether (4×90 mL). The organic layers are combined and the solvent removed by rotary evaporation. The product is dried under vacuum overnight.

### EXAMPLE 64

### Preparation of rac-Methadone (S)-Ketoprofenate from rac-Methadone Hydrochloride and (S)-Ketoprofen

rac-Methadone (S)-ketoprofen may be prepared using the following synthetic scheme. (S)-Ketoprofen (0.2543 g, 1.00 mmol) dissolved in a minimal volume of ethanol is combined with potassium hydroxide (0.05611 g, 1.00 mmol) dissolved in a minimal volume of ethanol and stirred for 1 hour. rac-Methadone hydrochloride (0.3459 g, 1.00 mmol) is dissolved in a minimal volume of water and combined with the ethanolic ketoprofenate solution. The resulting solution is stirred and the total volume reduced to approximately 30 mL. The concentrated solution is transferred to a separatory funnel and the desired product extracted with diethyl ether (4×90 mL). The organic layers are combined and the solvent removed by rotary evaporation. The product is dried under vacuum overnight.

### EXAMPLE 65

### Preparation of rac-Methadone Suprofenate from rac-Methadone Hydrochloride and Suprofen

rac-Methadone suprofenate may be prepared using the following synthetic scheme. Suprofen (0.2603 g, 1.00 mmol) dissolved in a minimal volume of ethanol is combined with potassium hydroxide (0.05611 g, 1.00 mmol) dissolved in a minimal volume of ethanol and stirred for 1 hour. rac-Methadone hydrochloride (0.3459 g, 1.00 mmol) is dissolved in a minimal volume of water and combined with the ethanolic suprofenate solution. The resulting solution is stirred and the total volume reduced to approximately 30 mL. The concentrated solution is transferred to a separatory funnel and the desired product extracted with diethyl ether (4×90 mL). The organic layers are combined and the solvent removed by rotary evaporation. The product is dried under vacuum overnight.

### EXAMPLE 66

### Preparation of rac-Methadone (S)-Flurbiprofenate from rac-Methadone Hydrochloride and (S)-Flurbiprofen

rac-Methadone (S)-flurbiprofen may be prepared using the following synthetic scheme. (S)-Flurbiprofen (0.2443 g, 1.00 mmol) dissolved in a minimal volume of ethanol is combined with potassium hydroxide (0.05611 g, 1.00 mmol) dissolved in a minimal volume of ethanol and stirred for 1 hour. rac-Methadone hydrochloride (0.3459 g, 1.00 mmol) is dissolved in a minimal volume of water and combined with the ethanolic flurbiprofenate solution. The resulting solution is stirred and the total volume reduced to approximately 30 mL. The concentrated solution is transferred to a separatory funnel and the desired product extracted with diethyl ether (4×90 mL). The organic layers are combined and the solvent removed by rotary evaporation. The product is dried under vacuum overnight.

### EXAMPLE 67

### Preparation of rac-Methadone Tolmetinate from rac-Methadone Hydrochloride and Tolmetin Sodium Dihydrate

rac-Methadone tolmetinate may be prepared using the following synthetic scheme. Aqueous solutions of tolmetin sodium dihydrate (0.3153 g, 1.00 mmol) and of rac-Methadone hydrochloride (0.3459 g, 1.00 mmol) are combined into a suitable flask and stirred for 60 minutes. The resulting solution is transferred to a separatory funnel and the desired product extracted with diethyl ether (4×90 mL). The organic layers are combined and the solvent removed by rotary evaporation. The product is dried under vacuum overnight.

### EXAMPLE 68

### Preparation of rac-Methadone Fenoprofenate from rac-Methadone Hydrochloride and Fenoprofen Calcium Trihydrate

rac-Methadone fenoprofenate may be prepared using the following synthetic scheme. Aqueous solutions of fenoprofen calcium trihydrate (0.2884 g, 0.50 mmol) and of rac-methadone hydrochloride (0.3459 g, 1.00 mmol) are combined into a suitable flask and stirred for 60 minutes. The resulting solution is transferred to a separatory funnel and the desired product extracted with diethyl ether (4×90 mL). The organic layers are combined and the solvent removed by rotary evaporation. The product is dried under vacuum overnight.

### EXAMPLE 69

### Preparation of rac-Methadone Oxaprozinate from rac-Methadone Hydrochloride and Oxaprozin

rac-Methadone oxaprozinate may be prepared using the following synthetic scheme. Oxaprozin (0.2933 g, 1.00 mmol) dissolved in a minimal volume of ethanol is combined with potassium hydroxide (0.05611 g, 1.00 mmol) dissolved in a minimal volume of ethanol and stirred for 1 hour. rac-Methadone hydrochloride (0.3459 g, 1.00 mmol) is dissolved in a minimal volume of water and combined with the ethanolic oxaprozinate solution. The resulting solution is stirred and the total volume reduced to approximately 30 mL. The concentrated solution is transferred to a separatory funnel and the desired product extracted with diethyl ether (4×90 mL). The organic layers are combined and the solvent removed by rotary evaporation. The product is dried under vacuum overnight.

### EXAMPLE 70

### Preparation of rac-Methadone Difunisalate from rac-Methadone Hydrochloride and Difunisal

rac-Methadone difunisalate may be prepared using the following synthetic scheme. Difunisal (0.2502 g, 1.00 mmol) dissolved in a minimal volume of ethanol is combined with potassium hydroxide (0.05611 g, 1.00 mmol) dissolved in a minimal volume of ethanol and stirred for 1 hour. rac-Methadone hydrochloride (0.3459 g, 1.00 mmol) is dissolved in a minimal volume of water and combined with the ethanolic difunisalate solution. The resulting solution is stirred and the total volume reduced to approximately 30 mL. The concentrated solution is transferred to a separatory funnel and the desired product extracted with diethyl ether (4×90 mL). The organic layers are combined and the solvent removed by rotary evaporation. The product is dried under vacuum overnight.

### EXAMPLE 71

### Preparation of rac-Methadone Loxoprofenate from rac-Methadone Hydrochloride and Loxoprofen

rac-Methadone loxoprofenate may be prepared using the following synthetic scheme. Loxoprofen (0.2463 g, 1.00 mmol) is dissolved in a minimal volume of ethanol and combined with potassium hydroxide (0.05611 g, 1.00 mmol) dissolved in a minimal volume of ethanol and stirred for 1 hour. rac-Methadone hydrochloride (0.3459 g, 1.00 mmol) is dissolved in a minimal volume of water and combined with the ethanolic loxoprofenate solution. The resulting solution is stirred and the total volume reduced to approximately 30 mL. The concentrated solution is transferred to a separatory funnel and the desired product extracted with diethyl ether (4×90 mL). The organic layers are combined and the solvent removed by rotary evaporation. The product is dried under vacuum overnight.

### EXAMPLE 72

### Preparation of 5R,9R,13S,14R-Hydrocodone Ibuprofenate from 5R,9R,13S,14R-Hydrocodone Bitartrate Hemipentahydrate and Ibuprofen

5R,9R,13S,14R-Hydrocodone ibuprofenate may be prepared using the following synthetic scheme. Ibuprofen (0.2063 g, 1.00 mmol) dissolved in a minimal volume of ethanol is combined with potassium hydroxide (0.05611 g, 1.00 mmol) dissolved in a minimal volume of ethanol and stirred for 1 hour. 5R,9R,13S,14R-hydrocodone bitartrate hemipentahydrate (0.4549 g, 1.00 mmol) is dissolved in a minimal volume of water and combined with the ethanolic ibuprofenate solution. The resulting solution is stirred and the total volume reduced to approximately 30 mL. The concentrated solution is transferred to a separatory funnel and the desired product extracted with diethyl ether (4×90 mL). The organic layers are combined and the solvent removed by rotary evaporation. The product is dried under vacuum overnight.

### EXAMPLE 73

### Preparation of 5R,9R,13S,14R-Hydrocodone Acetylsalicylate from 5R,9R,13S,14R-Hydrocodone Bitartrate Hemipentahydrate and Acetylsalicylic Acid

5R,9R,13S,14R-Hydrocodone acetylsalicylate may be prepared using the following synthetic scheme. Acetylsalicylic acid (0.3003 g, 1.00 mmol) dissolved in a minimal volume of ethanol is combined with potassium hydroxide (0.05611 g, 1.00 mmol) dissolved in a minimal volume of ethanol and stirred for 1 hour. 5R,9R,13S,14R-hydrocodone bitartrate hemipentahydrate (0.4945 g, 1.00 mmol) is dissolved in a minimal volume of water and combined with the ethanolic acetylsalicylate solution. The resulting solution is stirred and the total volume reduced to approximately 30 mL. The solution is transferred to a separatory funnel and the desired product extracted with diethyl ether (4×90 mL). The organic layers are combined and the solvent removed by rotary evaporation. The product is dried under vacuum overnight.

### EXAMPLE 74

### Preparation of 5R,9R,13S,14R-Hydrocodone Salicylate from 5R,9R,13S,14R-Hydrocodone Bitartrate Hemipentahydrate and Sodium Salicylate

5R,9R,13S,14R-Hydrocodone salicylate may be prepared using the following synthetic scheme. Aqueous solutions of sodium salicylate (0.1601 g, 1.00 mmol) and of 5R,9R,13S,14R-hydrocodone bitartrate hemipentahydrate (0.4945 g, 1.00 mmol) are combined into a suitable flask and stirred for 60 minutes. The resulting solution is transferred to a separatory funnel and the desired product extracted with diethyl ether (4×90 mL). The organic layers are combined and the solvent removed by rotary evaporation. The product is dried under vacuum overnight.

### EXAMPLE 75

### Preparation of 5R,9R,13S,14R-Hydrocodone Indomethacinate from 5R,9R,13S,14R-Hydrocodone Bitartrate Hemipentahydrate and Indomethacin

5R,9R,13S,14R-Hydrocodone indomethacinate may be prepared using the following synthetic scheme. Indomethacin (0.3578 g, 1.00 mmol) dissolved in a minimal volume of ethanol is combined with potassium hydroxide (0.05611 g, 1.00 mmol) dissolved in a minimal volume of ethanol and stirred for 1 hour. 5R,9R,13S,14R-Hydrocodone bitartrate hemipentahydrate (0.4945 g, 1.00 mmol) is dissolved in a minimal volume of water and combined with the ethanolic indomethacinate solution. The resulting solution is stirred and the total volume reduced to approximately 30 mL. The solution is transferred to a separatory funnel and the desired product extracted with diethyl ether (4×90 mL). The organic layers are combined and the solvent removed by rotary evaporation. The product is dried under vacuum overnight.

### EXAMPLE 76

### Preparation of 5R,9R,13S,14R-Hydrocodone Naproxenate from 5R,9R,13S,14R-Hydrocodone Bitartrate Hemipentahydrate and Naproxen Sodium

5R,9R,13S,14R-Hydrocodone naproxenate may be prepared using the following synthetic scheme. Aqueous solutions of naproxen sodium (0.2522 g, 1.00 mmol) and of 5R,9R,13S,14R-hydrocodone bitartrate hemipentahydrate (0.4945 g, 1.00 mmol) are combined into a suitable flask and stirred for 60 minutes. The resulting solution is transferred to a separatory funnel and the desired product extracted with diethyl ether (4×90 mL). The organic layers are combined and the solvent removed by rotary evaporation. The product is dried under vacuum overnight.

### EXAMPLE 77

### Preparation of 5R,9R,13S,14R-Hydrocodone Etodolate from 5R,9R,13S,14R-Hydrocodone Bitartrate Hemipentahydrate and Etodolac

5R,9R,13S,14R-Hydrocodone etodolate may be prepared using the following synthetic scheme. Etodolac (0.2874 g, 1.00 mmol) dissolved in a minimal volume of ethanol is combined with potassium hydroxide (0.05611 g, 1.00 mmol) dissolved in a minimal volume of ethanol and stirred for 1 hour. 5R,9R,13S,14R-Hydrocodone bitartrate hemipentahydrate (0.4945 g, 1.00 mmol) is dissolved in a minimal volume of water and combined with the ethanolic etodolate solution. The resulting solution is stirred and the total volume reduced to approximately 30 mL. The concentrated solution is transferred to a separatory funnel and the desired product extracted with diethyl ether (4×90 mL). The organic layers are combined and the solvent removed by rotary evaporation. The product is dried under vacuum overnight.

### EXAMPLE 78

### Preparation of 5R,9R,13S,14R-Hydrocodone Sulindate from 5R,9R,13S,14R-Hydrocodone Bitartrate Hemipentahydrate and Sulindac

R,9R,13S,14R-Hydrocodone sulindate may be prepared using the following synthetic scheme. Sulindac (0.3564 g, 1.00 mmol) dissolved in a minimal volume of ethanol is combined with potassium hydroxide (0.05611 g, 1.00 mmol) dissolved in a minimal amount of ethanol and stirred for 1 hour. 5R,9R,13S,14R-Hydrocodone bitartrate hemipentahydrate (0.4945 g, 1.00 mmol) is dissolved in a minimal volume of water and combined with the ethanolic sulindate solution. The resulting solution is stirred and the total volume reduced to approximately 30 mL. The concentrated solution is transferred to a separatory funnel and the desired product extracted with diethyl ether (4×90 mL). The organic layers are combined and the solvent removed by rotary evaporation. The product is dried under vacuum overnight.

### EXAMPLE 79

### Preparation of 5R,9R,13S,14R-Hydrocodone (S)-Ketoprofenate from 5R,9R,13S,14R-Hydrocodone Bitartrate Hemipentahydrate and (S)-Ketoprofen

5R,9R,13S,14R-Hydrocodone (S)-ketoprofenate may be prepared using the following synthetic scheme. (S)-Ketoprofen (0.2543 g, 1.00 mmol) dissolved in a minimal volume of ethanol is combined with potassium hydroxide (0.05611 g, 1.00 mmol) dissolved in a minimal volume of ethanol and stirred for 1 hour. 5R,9R,13S,14R-Hydrocodone bitartrate hemipentahydrate (0.4945 g, 1.00 mmol) is dissolved in a minimal volume of water and combined with the ethanolic ketoprofenate solution. The resulting solution is stirred and the total volume reduced to approximately 30 mL. The solution is transferred to a separatory funnel and the desired product extracted with diethyl ether (4×90 mL). The organic layers are combined and the solvent removed by rotary evaporation. The product is dried under vacuum overnight.

### EXAMPLE 80

### Preparation of 5R,9R,13S,14R-Hydrocodone Suprofenate from 5R,9R,13S,14R-Hydrocodone Bitartrate Hemipentahydrate and Suprofen

5R,9R,13S,14R-Hydrocodone suprofenate may be prepared using the following synthetic scheme. Suprofen (0.2603 g, 1.00 mmol) is dissolved in a minimal volume of ethanol and combined with potassium hydroxide (0.05611 g, 1.00 mmol) dissolved in a minimal volume of ethanol and stirred for 1 hour. 5R,9R,13S,14R-Hydrocodone bitartrate hemipentahydrate (0.4945 g, 1.00 mmol) is dissolved in a minimal volume of water and combined with the ethanolic suprofenate solution. The resulting solution is stirred and the total volume reduced to approximately 30 mL. The concentrated solution is transferred to a separatory funnel and the desired product extracted with diethyl ether (4×90 mL). The organic layers are combined and the solvent removed by rotary evaporation. The product is dried under vacuum overnight.

### EXAMPLE 81

### Preparation of 5R,9R,13S,14R-Hydrocodone (S)-Flurbiprofenate from 5R,9R,13S,14R-Hydrocodone Bitartrate Hemipentahydrate and (S)-Flurbiprofen

5R,9R,13S,14R-Hydrocodone (S)-flurbiprofenate may be prepared using the following synthetic scheme. (S)-Flurbiprofen (0.2443 g, 1.00 mmol) dissolved in a minimal volume of ethanol is combined with potassium hydroxide (0.05611 g, 1.00 mmol) dissolved in a minimal volume of ethanol and stirred for 1 hour. 5R,9R,13S,14R-Hydrocodone bitartrate hemipentahydrate (0.4945 g, 1.00 mmol) is dissolved in a minimal volume of water and combined with the ethanolic flurbiprofenate solution. The resulting solution is stirred and the total volume reduced to approximately 30 mL. The concentrated solution is transferred to a separatory funnel and the desired product extracted with diethyl ether (4×90 mL). The organic layers are combined and the solvent removed by rotary evaporation. The product is dried under vacuum overnight.

### EXAMPLE 82

### Preparation of 5R,9R,13S,14R-Hydrocodone Tolmetinate from 5R,9R,13S,14R-Hydrocodone Bitartrate Hemipentahydrate and Tolmetin Sodium Dihydrate

5R,9R,13S,14R-Hydrocodone tolmetinate may be prepared using the following synthetic scheme. Aqueous solutions of tolmetin sodium dihydrate (0.3153 g, 1.00 mmol) and of 5R,9R,13S,14R-Hydrocodone bitartrate hemipentahydrate (0.4945 g, 1.00 mmol) are combined into a suitable flask and stirred for 60 minutes The resulting solution is transferred to a separatory funnel and the desired product extracted with diethyl ether (4×90 mL). The organic layers are combined and the solvent removed by rotary evaporation. The product is dried under vacuum overnight.

### EXAMPLE 83

### Preparation of 5R,9R,13S,14R-Hydrocodone Fenoprofenate from 5R,9R,13S,14R-Hydrocodone Bitartrate Hemipentahydrate and Fenoprofen Calcium Trihydrate

5R,9R,13S,14R-Hydrocodone fenoprofenate may be prepared using the following synthetic scheme. Aqueous solutions of fenoprofen calcium trihydrate (0.2884 g, 0.50 mmol) and of 5R,9R,13S,14R-hydrocodone bitartrate hemipentahydrate (0.4945 g, 1.00 mmol) are combined into a suitable flask and stirred for 60 minutes. The resulting solution is transferred to a separatory funnel and the desired product extracted with diethyl ether (4×90 mL). The organic layers are combined and the solvent removed by rotary evaporation. The product is dried under vacuum overnight.

### EXAMPLE 84

### Preparation of 5R,9R,13S,14R-Hydrocodone Oxaprozinate from 5R,9R,13S,14R-Hydrocodone Bitartrate Hemipentahydrate and Oxaprozin

5R,9R,13S,14R-Hydrocodone oxaprozinate may be prepared using the following synthetic scheme. Oxaprozin (0.2933 g, 1.00 mmol) dissolved in a minimal volume of ethanol is combined with potassium hydroxide (0.05611 g, 1.00 mmol) dissolved in a minimal volume of ethanol and stirred for 1 hour. 5R, 9R, 13S, 14R-Hydrocodone bitartrate hemipentahydrate (0.4945 g, 1.00 mmol) is dissolved in a minimal volume of water and combined with the ethanolic oxaprozinate solution. The resulting solution is stirred and the total volume reduced to approximately 30 mL. The concentrated solution is transferred to a separatory funnel and the desired product extracted with diethyl ether (4×90 mL). The organic layers are combined and the solvent removed by rotary evaporation. The product is dried under vacuum overnight.

### EXAMPLE 85

### Preparation of 5R,9R,13S,14R-Hydrocodone Difunisalate from 5R,9R,13S,14R-Hydrocodone Bitartrate Hemipentahydrate and Difunisal

5R,9R,13S,14R-Hydrocodone difunisalate may be prepared using the following synthetic scheme. Difunisal (0.2502 g, 1.00 mmol) dissolved in a minimal volume of ethanol is combined with potassium hydroxide (0.05611 g, 1.00 mmol) dissolved in a minimal volume of ethanol and stirred for 1 hour. 5R,9R,13S,14R-Hydrocodone bitartrate hemipentahydrate (0.4945 g, 1.00 mmol) is dissolved in a minimal volume of water and combined with the ethanolic difunisalate solution. The resulting solution is stirred and the total volume reduced to approximately 30 mL. The concentrated solution is transferred to a separatory funnel and the desired product extracted with diethyl ether (4×90 mL). The organic layers are combined and the solvent removed by rotary evaporation. The product is dried under vacuum overnight.

### EXAMPLE 86

### Preparation of 5R,9R,13S,14R-Hydrocodone Loxoprofenate from 5R,9R,13S,14R-Hydrocodone Bitartrate Hemipentahydrate and Loxoprofen

5R,9R,13S,14R-Hydrocodone loxoprofenate may be prepared using the following synthetic scheme. Loxoprofen (0.2463 g, 1.00 mmol) dissolved in a minimal volume of ethanol is combined with potassium hydroxide (0.05611 g, 1.00 mmol) dissolved in a minimal volume of ethanol and stirred for 1 hour. 5R,9R,13S,14R-Hydrocodone bitartrate hemipentahydrate (0.4945 g, 1.00 mmol) is dissolved in a minimal volume of water and combined with the ethanolic loxoprofenate solution. The resulting solution is stirred and the total volume reduced to approximately 30 mL. The concentrated solution is transferred to a separatory funnel and the desired product extracted with diethyl ether (4×90 mL). The organic layers are combined and the solvent removed by rotary evaporation. The product is dried under vacuum overnight.

### EXAMPLE 87

### Preparation of 5R,6S,9R,13S,14R-Codeine Ibuprofenate from 5R,6S,9R,13S,14R-Codeine Sulfate and Ibuprofen

5R,6S,9R,13S,14R-Codeine ibuprofenate may be prepared using the following synthetic scheme. Ibuprofen (0.2063 g, 1.00 mmol) dissolved in a minimal volume of ethanol and combined with potassium hydroxide (0.05611 g, 1.00 mmol) dissolved in a minimal volume of ethanol and stirred for 1 hour. Codeine sulfate (0.3484 g, 0.50 mmol) is dissolved in a minimal volume of water and combined with the ethanolic ibuprofenate solution. The resulting solution is stirred and the total volume reduced to approximately 30 mL. The concentrated solution is transferred to a separatory funnel and the desired product extracted with diethyl ether (4×90 mL). The organic layers are combined and the solvent removed by rotary evaporation. The product is dried under vacuum overnight.

### EXAMPLE 88

### Preparation of 5R,6S,9R,13S,14R-Codeine Acetylsalicylate from 5R,6S,9R,13S,14R-Codeine Sulfate and Acetylsalicylic Acid

5R,6S,9R,13S,14R-Codeine acetylsalicylate may be prepared using the following synthetic scheme. Acetylsalicylic acid (0.3003 g, 1.00 mmol) is dissolved in a minimal volume of ethanol and combined with potassium hydroxide (0.05611 g, 1.00 mmol) dissolved in a minimal volume of ethanol and stirred for 1 hour. Codeine sulfate (0.3484 g, 0.50 mmol) is dissolved in a minimal volume of water and combined with the ethanolic acetylsalicylate solution. The resulting solution is stirred and the total volume reduced to approximately 30 mL. The concentrated solution is transferred to a separatory funnel and the desired product extracted with diethyl ether (4×90 mL). The organic layers are combined and the solvent removed by rotary evaporation. The product is dried under vacuum overnight.

### EXAMPLE 89

### Preparation of 5R,6S,9R,13S,14R-Codeine Salicylate from 5R,6S,9R,13S,14R-Codeine Sulfate and Sodium Salicylate

5R,6S,9R,13S,14R-Codeine salicylate may be prepared using the following synthetic scheme. Aqueous solutions of sodium salicylate (0.1601 g, 1.00 mmol) and of codeine sulfate (0.3484 g, 0.50 mmol) are combined into a suitable flask and stirred for 60 minutes. The resulting solution is transferred to a separatory funnel and the desired product extracted with diethyl ether (4×90 mL). The organic layers are combined and the solvent removed by rotary evaporation. The product is dried under vacuum overnight.

### EXAMPLE 90

### Preparation of 5R,6S,9R,13S,14R-Codeine Indomethacinate from 5R,6S,9R,13S,14R-Codeine Sulfate and Indomethacin

5R,6S,9R,13S,14R-Codeine indomethacinate may be prepared using the following synthetic scheme. Indomethacin (0.3578 g, 1.00 mmol) is dissolved in a minimal volume of ethanol and combined with potassium hydroxide (0.05611 g, 1.00 mmol) dissolved in a minimal volume of ethanol and stirred for 1 hour. Codeine sulfate (0.3484 g, 0.50 mmol) is dissolved in a minimal volume of water and combined with the ethanolic indomethacinate solution. The resulting solution is stirred and the total volume reduced to approximately 30 mL. The concentrated solution is transferred to a separatory funnel and the desired product extracted with diethyl ether (4×90 mL). The organic layers are combined and the solvent removed by rotary evaporation. The product is dried under vacuum overnight.

### EXAMPLE 91

### Preparation of 5R,6S,9R,13S,14R-Codeine Naproxenate from 5R,6S,9R,13S,14R-Codeine Sulfate and Naproxen Sodium

5R,6S,9R,13S,14R-Codeine naproxenate may be prepared using the following synthetic scheme. Aqueous solutions of naproxen sodium (0.2522 g, 1.00 mmol) and of codeine sulfate (0.3484 g, 0.50 mmol) are combined into a suitable flask and stirred for 60 minutes. The resulting solution is transferred to a separatory funnel and the desired product extracted with diethyl ether (4×90 mL). The organic layers are combined and the solvent removed by rotary evaporation. The product is dried under vacuum overnight.

### EXAMPLE 92

### Preparation of 5R,6S,9R,13S,14R-Codeine Etodolate from 5R,6S,9R,13S,14R-Codeine Sulfate and Etodolac

5R,6S,9R,13S,14R-Codeine etodolate may be prepared using the following synthetic scheme. Etodolac (0.2874 g, 1.00 mmol) dissolved in a minimal volume of ethanol is combined with potassium hydroxide (0.05611 g, 1.00 mmol) dissolved in a minimal volume of ethanol and stirred for 1 hour. Codeine sulfate (0.3484 g, 0.50 mmol) is dissolved in a minimal volume of water and combined with the ethanolic etodolate solution. The resulting solution is stirred and the total volume reduced to approximately 30 mL. The concentrated solution is transferred to a separatory funnel and the desired product extracted with diethyl ether (4×90 mL). The organic layers are combined and the solvent removed by rotary evaporation. The product is dried under vacuum overnight.

### EXAMPLE 93

### Preparation of 5R,6S,9R,13S,14R-Codeine Sulindate from 5R,6S,9R,13S,14R-Codeine Sulfate and Sulindac

5R,6S,9R,13S,14R-Codeine sulindate may be prepared using the following synthetic scheme. Sulindac (0.3564 g, 1.00 mmol) dissolved in a minimal volume of ethanol is combined with potassium hydroxide (0.05611 g, 1.00 mmol) dissolved in a minimal volume of ethanol and stirred for 1 hour. Codeine sulfate (0.3484 g, 0.50 mmol) is dissolved in a minimal volume of water and combined with the ethanolic sulindate solution. The resulting solution is stirred and the total volume reduced to approximately 30 mL. The concentrated solution is transferred to a separatory funnel and the desired product extracted with diethyl ether (4×90 mL). The organic layers are combined and the solvent removed by rotary evaporation. The product is dried under vacuum overnight.

### EXAMPLE 94

### Preparation of 5R,6S,9R,13S,14R-Codeine (S)-Ketoprofenate from 5R,6S,9R,13S,14R-Codeine Sulfate and (S)-Ketoprofen

5R,6S,9R,13S,14R-Codeine (S)-ketoprofenate may be prepared using the following synthetic scheme. (S)-Ketoprofen (0.2543 g, 1.00 mmol) dissolved in a minimal volume of ethanol and combined with potassium hydroxide (0.05611 g, 1.00 mmol) dissolved in a minimal volume of ethanol and stirred for 1 hour. Codeine sulfate (0.3484 g, 0.50 mmol) is dissolved in a minimal volume of water and combined with the ethanolic ketoprofenate solution. The resulting solution is stirred and the total volume reduced to approximately 30 mL. The concentrated solution is transferred to a separatory funnel and the desired product extracted with diethyl ether (4×90 mL). The organic layers are combined and the solvent removed by rotary evaporation. The product is dried under vacuum overnight.

### EXAMPLE 95

### Preparation of 5R,6S,9R,13S,14R-Codeine Suprofenate from 5R,6S,9R,13S,14R-Codeine Sulfate and Suprofen

5R,6S,9R,13S,14R-Codeine suprofenate may be prepared using the following synthetic scheme. Suprofen (0.2603 g, 1.00 mmol) dissolved in a minimal volume of ethanol is combined with potassium hydroxide (0.05611 g, 1.00 mmol) dissolved in a minimal volume of ethanol and stirred for 1 hour. Codeine sulfate (0.3484 g, 0.50 mmol) is dissolved in a minimal volume of water and combined with the ethanolic suprofenate solution. The resulting solution is stirred and the total volume reduced to approximately 30 mL. The condensed solution is transferred to a separatory funnel and the desired product extracted with diethyl ether (4×90 mL). The organic layers are combined and the solvent removed by rotary evaporation. The product is dried under vacuum overnight.

### EXAMPLE 96

### Preparation of 5R,6S,9R,13S,14R-Codeine (S)-Flurbiprofenate from 5R,6S,9R,13S,14R-Codeine Sulfate and (S)-Flurbiprofen

5R,6S,9R,13S,14R-Codeine (S)-flurbiprofenate may be prepared using the following synthetic scheme. (S)-Flurbiprofen (0.2443 g, 1.00 mmol) dissolved in a minimal volume of ethanol is combined with potassium hydroxide (0.05611 g, 1.00 mmol) dissolved in a minimal volume of ethanol and stirred for 1 hour. Codeine sulfate (0.3484 g, 0.50 mmol) is dissolved in a minimal volume of water and combined with the ethanolic flurbiprofenate solution. The resulting solution is stirred and the total volume reduced to approximately 30 mL. The concentrated solution is transferred to a separatory funnel and the desired product extracted with diethyl ether (4×90 mL). The organic layers are combined and the solvent removed by rotary evaporation. The product is dried under vacuum overnight.

### EXAMPLE 97

### Preparation of 5R,6S,9R,13S,14R-Codeine Tolmetinate from 5R,6S,9R,13S,14R-Codeine Sulfate and Tolmetin Sodium Dihydrate

5R,6S,9R,13S,14R-Codeine tolmetinate may be prepared using the following synthetic scheme. Aqueous solutions of tolmetin sodium dihydrate (0.3153 g, 1.00 mmol) and of codeine sulfate (0.3484 g, 0.50 mmol) are combined into a suitable flask and stirred for 60 minutes. The resulting solution is transferred to a separatory funnel and the desired product extracted with diethyl ether (4×90 mL). The organic layers are combined and the solvent removed by rotary evaporation. The product is dried under vacuum overnight.

### EXAMPLE 98

### Preparation of 5R,6S,9R,13S,14R-Codeine Fenoprofenate from 5R,6S,9R,13S,14R-Codeine Sulfate and Fenoprofen Calcium Trihydrate

5R,6S,9R,13S,14R-Codeine fenoprofenate may be prepared using the following synthetic scheme. Aqueous solutions of fenoprofen calcium trihydrate (0.2884 g, 0.50 mmol) and of codeine sulfate (0.3484 g, 0.50 mmol) are combined into a suitable flask and stirred for 60 minutes. The resulting solution is transferred to a separatory funnel and the desired product extracted with diethyl ether (4×90 mL). The organic layers are combined and the solvent removed by rotary evaporation. The product is dried under vacuum overnight.

### EXAMPLE 99

### Preparation of 5R,6S,9R,13S,14R-Codeine Oxaprozinate from 5R,6S,9R,13S,14R-Codeine Sulfate and Oxaprozin

5R,6S,9R,13S,14R-Codeine oxaprozinate may be prepared using the following synthetic scheme. Oxaprozin (0.2933 g, 1.00 mmol) dissolved in a minimal volume of ethanol is combined with potassium hydroxide (0.05611 g, 1.00 mmol) dissolved in a minimal volume of ethanol and stirred for 1 hour. Codeine sulfate (0.3484 g, 0.50 mmol) is dissolved in a minimal volume of water and combined with the ethanolic oxaprozinate solution. The resulting solution is stirred and the total volume reduced to approximately 30 mL. The concentrated solution is transferred to a separatory funnel and the desired product extracted with diethyl ether (4×90 mL). The organic layers are combined and the solvent removed by rotary evaporation. The product is dried under vacuum overnight.

### EXAMPLE 100

### Preparation of 5R,6S,9R,13S,14R-Codeine Difunisalate from 5R,6S,9R,13S,14R-Codeine Sulfate and Difunisal

5R,6S,9R,13S,14R-Codeine difunisalate may be prepared using the following synthetic scheme. Difunisal (0.2502 g, 1.00 mmol) dissolved in a minimal volume of ethanol is combined with potassium hydroxide (0.05611 g, 1.00 mmol) dissolved in a minimal volume of ethanol and stirred for 1 hour. Codeine sulfate (0.3484 g, 0.50 mmol) is dissolved in a minimal volume of water and combined with the ethanolic difunisalate solution. The resulting solution is stirred and the total volume reduced to approximately 30 mL. The concentrated solution is transferred to a separatory funnel and the desired product extracted with diethyl ether (4×90 mL). The organic layers are combined and the solvent removed by rotary evaporation. The product is dried under vacuum overnight.

### EXAMPLE 101

### Preparation of 5R,6S,9R,13S,14R-Codeine Loxoprofenate from 5R,6S,9R,13S,14R-Codeine Sulfate and Loxoprofen

5R,6S,9R,13S,14R-Codeine loxoprofenate may be prepared using the following synthetic scheme. Loxoprofen (0.2463 g, 1.00 mmol) dissolved in a minimal volume of ethanol is combined with potassium hydroxide (0.05611 g, 1.00 mmol) dissolved in a minimal volume of ethanol and stirred for 1 hour. Codeine sulfate (0.3484 g, 0.50 mmol) is dissolved in a minimal volume of water and combined with the ethanolic loxoprofenate solution. The resulting solution is stirred and the total volume reduced to approximately 30 mL. The concentrated solution is transferred to a separatory funnel and the desired product extracted with diethyl ether (4×90 mL). The organic layers are combined and the solvent removed by rotary evaporation. The product is dried under vacuum overnight.

### EXAMPLE 102

### Preparation of 5R,6S,9R,13S,14R-Morphine Ibuprofenate from 5R,6S,9R,13S,14R-Morphine Sulfate Pentahydrate and Ibuprofen

5R,6S,9R,13S,14R-Morphine ibuprofenate may be prepared using the following synthetic scheme. Ibuprofen (0.2063 g, 1.00 mmol) dissolved in a minimal volume of ethanol is combined with potassium hydroxide (0.05611 g, 1.00 mmol) dissolved in a minimal volume of ethanol and stirred for 1 hour. Morphine sulfate pentahydrate (0.3794 g, 0.50 mmol) is dissolved in a minimal volume of water and combined with the ethanolic ibuprofenate solution. The resulting solution is stirred and the total volume reduced to approximately 30 mL. The solution is transferred to a separatory funnel and the desired product extracted with diethyl ether (4×90 mL). The organic layers are combined and the solvent removed by rotary evaporation. The product is dried under vacuum overnight.

### EXAMPLE 103

### Preparation of 5R,6S,9R,13S,14R-Morphine Acetylsalicylate from 5R,6S,9R,13S,14R-Morphine Sulfate Pentahydrate and Acetylsalicylic Acid

5R,6S,9R,13S,14R-Morphine acetylsalicylate may be prepared using the following synthetic scheme. Acetylsalicylic acid (0.3003 g, 1.00 mmol) dissolved in a minimal volume of ethanol is combined with potassium hydroxide (0.05611 g, 1.00 mmol) dissolved in a minimal volume of ethanol and stirred for 1 hour. Morphine sulfate pentahydrate (0.3794 g, 0.50 mmol) is dissolved in a minimal volume of water and combined with the ethanolic acetylsalicylate solution. The resulting solution is stirred and the total volume reduced to approximately 30 mL. The concentrated solution is transferred to a separatory funnel and the desired product extracted with diethyl ether (4×90 mL). The organic layers are combined and the solvent removed by rotary evaporation. The product is dried under vacuum overnight.

### EXAMPLE 104

### Preparation of 5R,6S,9R,13S,14R-Morphine Salicylate from 5R,6S,9R,13S,14R-Morphine Sulfate Pentahydrate and Sodium Salicylate

5R,6S,9R,13S,14R-Morphine salicylate may be prepared using the following synthetic scheme. Aqueous solutions of sodium salicylate (0.1601 g, 1.00 mmol) and of morphine sulfate pentahydrate (0.3794 g, 0.50 mmol) are combined into a suitable flask and stirred for 60 minutes. The resulting solution is transferred to a separatory funnel and the desired product extracted with diethyl ether (4×90 mL). The organic layers are combined and the solvent removed by rotary evaporation. The product is dried under vacuum overnight.

### EXAMPLE 105

### Preparation of 5R,6S,9R,13S,14R-Morphine Indomethacinate from 5R,6S,9R,13S,14R-Morphine Sulfate Pentahydrate and Indomethacin

5R,6S,9R,13S,14R-Morphine indomethacinate may be prepared using the following synthetic scheme. Indomethacin (0.3578 g, 1.00 mmol) dissolved in a minimal volume of ethanol is combined with potassium hydroxide (0.05611 g, 1.00 mmol) dissolved in a minimal volume of ethanol and stirred for 1 hour. Morphine sulfate pentahydrate (0.3794 g, 0.50 mmol) is dissolved in a minimal volume of water and combined with the ethanolic indomethacinate solution. The resulting solution is stirred and the total volume reduced to approximately 30 mL. The solution is transferred to a separatory funnel and the desired product extracted with diethyl ether (4×90 mL). The organic layers are combined and the solvent removed by rotary evaporation. The product is dried under vacuum overnight.

### EXAMPLE 106

### Preparation of 5R,6S,9R,13S,14R-Morphine Naproxenate from 5R,6S,9R,13S,14R-Morphine Sulfate Pentahydrate and Naproxen Sodium

5R,6S,9R,13S,14R-Morphine naproxenate may be prepared using the following synthetic scheme. Aqueous solutions of naproxen sodium (0.2522 g, 1.00 mmol) and of morphine sulfate pentahydrate (0.3794 g, 0.50 mmol) are combined into a suitable flask and stirred for 60 minutes. The resulting solution is transferred to a separatory funnel and the desired product extracted with diethyl ether (4×90 mL). The organic layers are combined and the solvent removed by rotary evaporation. The product is dried under vacuum overnight.

### EXAMPLE 107

### Preparation of 5R,6S,9R,13S,14R-Morphine Etodolate from 5R,6S,9R,13S,14R-Morphine Sulfate Pentahydrate and Etodolac

5R,6S,9R,13S,14R-Morphine etodolate may be prepared using the following synthetic scheme. Etodolac (0.2874 g, 1.00 mmol) dissolved in a minimal volume of ethanol is combined with potassium hydroxide (0.05611 g, 1.00 mmol) dissolved in a minimal volume of ethanol and stirred for 1 hour. Morphine sulfate pentahydrate (0.3794 g, 0.50 mmol) is dissolved in a minimal volume of water and combined with the ethanolic etodolate solution. The resulting solution is stirred and the total volume reduced to approximately 30 mL. The concentrated solution is transferred to a separatory funnel and the desired product extracted with diethyl ether (4×90 mL). The organic layers are combined and the solvent removed by rotary evaporation.

### EXAMPLE 108

### Preparation of 5R,6S,9R,13S,14R-Morphine Sulindate from 5R,6S,9R,13S,14R-Morphine Sulfate Pentahydrate and Sulindac

5R,6S,9R,13S,14R-Morphine sulindate may be prepared using the following synthetic scheme. Sulindac (0.3564 g, 1.00 mmol) dissolved in a minimal volume of ethanol is combined with potassium hydroxide (0.05611 g, 1.00 mmol) dissolved in a minimal volume of ethanol and stirred for 1 hour. Morphine sulfate pentahydrate (0.3794 g, 0.50 mmol) is dissolved in a minimal volume of water and combined with the ethanolic sulindate solution. The resulting solution is stirred and the total volume is reduced to approximately 30 mL. The concentrated solution is transferred to a separatory funnel and the desired product extracted with diethyl ether (4×90 mL). The organic layers are combined and the solvent removed by rotary evaporation. The product is dried under vacuum overnight.

### EXAMPLE 109

### Preparation of 5R,6S,9R,13S,14R-Morphine (S)-Ketoprofenate from 5R,6S,9R,13S,14R-Morphine Sulfate Pentahydrate and (S)-Ketoprofen

5R,6S,9R,13S,14R-Morphine (S)-ketoprofenate may be prepared using the following synthetic scheme. (S)-Ketoprofen (0.2543 g, 1.00 mmol) dissolved in a minimal volume of ethanol is combined with potassium hydroxide (0.05611 g, 1.00 mmol) dissolved in a minimal volume of ethanol and stirred for 1 hour. Morphine sulfate pentahydrate (0.3794 g, 0.50 mmol) is dissolved in a minimal volume of water and combined with the ethanolic ketoprofenate solution. The resulting solution is stirred and the total volume is reduced to approximately 30 mL. The concentrated solution is transferred to a separatory funnel and the desired product extracted with diethyl ether (4×90 mL). The organic layers are combined and the solvent removed by rotary evaporation. The product is dried under vacuum overnight.

### EXAMPLE 110

### Preparation of 5R,6S,9R,13S,14R-Morphine Suprofenate from 5R,6S,9R,13S,14R-Morphine Sulfate Pentahydrate and Suprofen

5R,6S,9R,13S,14R-Morphine suprofenate may be prepared using the following synthetic scheme. Suprofen (0.2603 g, 1.00 mmol) dissolved in a minimal volume of ethanol is combined with potassium hydroxide (0.05611 g, 1.00 mmol) dissolved in a minimal volume of ethanol and stirred for 1 hour. Morphine sulfate pentahydrate (0.3794 g, 0.50 mmol) is dissolved in a minimal volume of water and the ethanolic suprofenate solution. The resulting solution is stirred and the total volume reduced to approximately 30 mL. The concentrated solution is transferred to a separatory funnel and the desired product extracted with diethyl ether (4×90 mL). The organic layers are combined and the solvent removed by rotary evaporation. The product is dried under vacuum overnight.

### EXAMPLE 111

### Preparation of 5R,6S,9R,13S,14R-Morphine (S)-Flurbiprofenate from 5R,6S,9R,13S,14R-Morphine Sulfate Pentahydrate and (S)-Flurbiprofen

5R,6S,9R,13S,14R-Morphine (S)-flurbiprofenate may be prepared using the following synthetic scheme. (S)-Flurbiprofen (0.2443 g, 1.00 mmol) dissolved in a minimal volume of ethanol is combined with potassium hydroxide (0.05611 g, 1.00 mmol) dissolved in a minimal volume of ethanol and stirred for 1 hour. Morphine sulfate pentahydrate (0.3794 g, 0.50 mmol) is dissolved in a minimal volume of water and combined with the ethanolic flurbiprofenate solution. The resulting solution is stirred and the total volume reduced to approximately 30 mL. The concentrated solution is transferred to a separatory funnel and the desired product extracted with diethyl ether (4×90 mL). The organic layers are combined and the solvent removed by rotary evaporation. The product is dried under vacuum overnight.

### EXAMPLE 112

### Preparation of 5R,6S,9R,13S,14R-Morphine Tolmetinate from 5R,6S,9R,13S,14R-Morphine Sulfate Pentahydrate and Tolmetin Sodium Dihydrate

5R,6S,9R,13S,14R-Morphine tolmetinate may be prepared using the following synthetic scheme. Aqueous solutions of tolmetin sodium dihydrate (0.3153 g, 1.00 mol) and of Morphine sulfate pentahydrate (0.3794 g, 0.50 mmol) are combined into a suitable flask and stirred for 60 minutes. The resulting solution is transferred to a separatory funnel and the desired product extracted with diethyl ether (4×90 mL). The organic layers are combined and the solvent removed by rotary evaporation. The product is dried under vacuum overnight.

### EXAMPLE 113

### Preparation of 5R,6S,9R,13S,14R-Morphine Fenoprofenate from 5R,6S,9R,13S,14R-Morphine Sulfate Pentahydrate and Fenoprofen Calcium Trihydrate

5R,6S,9R,13S,14R-Morphine fenoprofenate may be prepared using the following synthetic scheme. Aqueous solutions of fenoprofen calcium trihydrate (0.2884 g, 0.50 mmol) and of morphine sulfate pentahydrate (0.3794 g, 0.50 mmol) are combined into a suitable flask and stirred for 60 minutes. The resulting solution is transferred to a separatory funnel and the desired product extracted with diethyl ether (4×90 mL). The organic layers are combined and the solvent removed by rotary evaporation. The product is dried under vacuum overnight.

### EXAMPLE 114

### Preparation of 5R,6S,9R,13S,14R-Morphine Oxaprozinate from 5R,6S,9R,13S,14R-Morphine Sulfate Pentahydrate and Oxaprozin

5R,6S,9R,13S,14R-Morphine oxaprozinate may be prepared using the following synthetic scheme. Oxaprozin (0.2933 g, 1.00 mmol) dissolved in a minimal volume of ethanol is combined with potassium hydroxide (0.05611 g, 1.00 mmol) dissolved in a minimal volume of ethanol and stirred for 1 hour. Morphine sulfate pentahydrate (0.3794 g, 0.50 mmol) is dissolved in a minimal volume of water and combined with the ethanolic oxaprozinate solution. The resulting solution is stirred and the total volume reduced to approximately 30 mL. The concentrated solution is transferred to a separatory funnel and the desired product extracted with diethyl ether (4×90 mL). The organic layers are combined and the solvent removed by rotary evaporation. The product is dried under vacuum overnight.

### EXAMPLE 115

### Preparation of 5R,6S,9R,13S,14R-Morphine Difunisalate from 5R,6S,9R,13S,14R-Morphine Sulfate Pentahydrate and Difunisal

5R,6S,9R,13S,14R-Morphine difunisalate may be prepared using the following synthetic scheme. Difunisal (0.2502 g, 1.00 mmol) dissolved in a minimal volume of ethanol is combined with potassium hydroxide (0.05611 g, 1.00 mmol) dissolved in a minimal volume of ethanol and stirred for 1 hour. Morphine sulfate pentahydrate (0.3794 g, 0.50 mmol) is dissolved in a minimal volume of water and combined with the ethanolic difunisalate solution. The resulting solution is stirred and the total volume reduced to approximately 30 mL. The concentrated solution is transferred to a separatory funnel and the desired product extracted with diethyl ether (4×90 mL). The organic layers are combined and the solvent removed by rotary evaporation. The product is dried under vacuum overnight.

### EXAMPLE 116

### Preparation of 5R,6S,9R,13S,14R-Morphine Loxoprofenate from 5R,6S,9R,13S,14R-Morphine Sulfate Pentahydrate and Loxoprofen

5R,6S,9R,13S,14R-Morphine loxoprofenate may be prepared using the following synthetic scheme. Loxoprofen (0.2463 g, 1.00 mmol) dissolved in a minimal volume of ethanol is combined with potassium hydroxide (0.05611 g, 1.00 mmol) dissolved in a minimal volume of ethanol and stirred for 1 hour. Morphine sulfate pentahydrate (0.3794 g, 0.50 mmol) is dissolved in a minimal volume of water and combined with the ethanolic loxoprofenate solution. The resulting solution is stirred and the total volume reduced to approximately 30 mL. The concentrated solution is transferred to a separatory funnel and the desired product extracted with diethyl ether (4×90 mL). The organic layers are combined and the solvent removed by rotary evaporation. The product is dried under vacuum overnight.

### EXAMPLE 117

### Preparation of Levorphanol Ibuprofenate from Levorphanol Tartrate Dihydrate and Ibuprofen

Levorphanol ibuprofenate may be prepared using the following synthetic scheme. Ibuprofen (0.2063 g, 1.00 mmol) dissolved in a minimal volume of ethanol is combined with potassium hydroxide (0.05611 g, 1.00 mmol) dissolved in a minimal volume of ethanol and stirred for 1 hour. A solution of (l)-3-hydroxy-N-methylmorphinan (herein referred to as levorphanol) tartrate dihydrate (0.4435 g, 1.00 mmol) is prepared in a minimal volume of water and combined with the ethanolic ibuprofenate solution. The resulting solution is stirred and the total volume reduced to approximately 30 mL. The concentrated solution is transferred to a separatory funnel and the desired product extracted with diethyl ether (4×90 mL). The organic layers are combined and the solvent removed by rotary evaporation. The product is dried under vacuum overnight.

### EXAMPLE 118

### Preparation of Levorphanol Acetylsalicylate from Levorphanol Tartrate Dihydrate and Acetylsalicylic Acid

Levorphanol acetylsalicylate may be prepared using the following synthetic scheme. Acetylsalicylic acid (0.3003 g, 1.00 mmol) dissolved in a minimal volume of ethanol is combined with potassium hydroxide (0.05611 g, 1.00 mmol) dissolved in a minimal volume of ethanol and stirred for 1 hour. Levorphanol tartrate dihydrate (0.4435 g, 1.00 mmol) is dissolved in a minimal volume of water and combined with the ethanolic acetylsalicylate solution. The resulting solution is stirred and the total volume reduced to approximately 30 mL. The concentrated solution is transferred to a separatory funnel and the desired product extracted with diethyl ether (4×90 mL). The organic layers are combined and the solvent removed by rotary evaporation. The product is dried under vacuum overnight.

### EXAMPLE 119

### Preparation of Levorphanol Salicylate from Levorphanol Tartrate Dihydrate and Sodium Salicylate

Levorphanol salicylate may be prepared using the following synthetic scheme. Aqueous solutions of sodium salicylate (0.1601 g, 1.00 mmol) and of levorphanol tartrate dihydrate (0.4435 g, 1.00 mmol) are combined into a suitable flask and stirred for 60 minutes. The resulting solution is transferred to a separatory funnel and the desired product extracted with diethyl ether (4×90 mL). The organic layers are combined and the solvent removed by rotary evaporation. The product is dried under vacuum overnight.

### EXAMPLE 120

### Preparation of Levorphanol Indomethacinate from Levorphanol Tartrate Dihydrate and Indomethacin

Levorphanol indomethacinate may be prepared using the following synthetic scheme. Indomethacin (0.3578 g, 1.00 mmol) dissolved in a minimal volume of ethanol is combined with potassium hydroxide (0.05611 g, 1.00 mmol) dissolved in a minimal volume of ethanol and stirred for 1 hour. Levorphanol tartrate dihydrate (0.4435 g, 1.00 mmol) is dissolved in a minimal volume of water and combined with the ethanolic indomethacinate solution. The resulting solution is stirred and the total volume reduced to approximately 30 mL. The concentrated solution is transferred to a separatory funnel and the desired product extracted with diethyl ether (4×90 mL). The organic layers are combined and the solvent removed by rotary evaporation. The product is dried under vacuum overnight.

### EXAMPLE 121

### Preparation of Levorphanol Naproxenate from Levorphanol Tartrate Dihydrate and Naproxen Sodium

Levorphanol naproxenate may be prepared using the following synthetic scheme. Aqueous solutions of naproxen sodium (0.2522 g, 1.00 mmol) and of levorphanol tartrate dihydrate (0.4435 g, 1.00 mmol) are combined into a suitable flask and stirred for 60 minutes. The resulting solution is transferred to a separatory funnel and the desired product extracted with diethyl ether (4×90 mL). The organic layers are combined and the solvent removed by rotary evaporation. The product is dried under vacuum overnight.

### EXAMPLE 122

### Preparation of Levorphanol Etodolate from Levorphanol Tartrate Dihydrate and Etodolac

Levorphanol etodolate may be prepared using the following synthetic scheme. Etodolac (0.2874 g, 1.00 mmol) dissolved in a minimal volume of ethanol is combined with potassium hydroxide (0.05611 g, 1.00 mmol) dissolved in a minimal volume of ethanol and stirred for 1 hour. Levorphanol tartrate dihydrate (0.4435 g, 1.00 mmol) is dissolved in a minimal volume of water and combined with the ethanolic etodolate solution. The resulting solution is stirred and the total volume reduced to approximately 30 mL. The concentrated solution is transferred to a separatory funnel and the desired product extracted with diethyl ether (4×90 mL). The organic layers are combined and the solvent removed by rotary evaporation. The product is dried under vacuum overnight.

### EXAMPLE 123

### Preparation of Levorphanol Sulindate from Levorphanol Tartrate Dihydrate and Sulindac

Levorphanol sulindate may be prepared using the following synthetic scheme. Sulindac (0.3564 g, 1.00 mmol) dissolved in a minimal volume of ethanol is combined with potassium hydroxide (0.05611 g, 1.00 mmol) dissolved in a minimal volume of ethanol and stirred for 1 hour. Levorphanol tartrate dihydrate (0.4435 g, 1.00 mmol) is dissolved in a minimal volume of water and combined with the ethanolic sulindate solution. The resulting solution is stirred and the total volume reduced to approximately 30 mL. The concentrated solution is transferred to a separatory funnel and the desired product extracted with diethyl ether (4×90 mL). The organic layers are combined and the solvent removed by rotary evaporation. The product is dried under vacuum overnight.

### EXAMPLE 124

### Preparation of Levorphanol (S)-Ketoprofenate from Levorphanol Tartrate Dihydrate and (S)-Ketoprofen

Levorphanol (S)-ketoprofenate may be prepared using the following synthetic scheme. (S)-Ketoprofen (0.2543 g, 1.00 mmol) dissolved in a minimal volume of ethanol is combined with potassium hydroxide (0.05611 g, 1.00 mmol) dissolved in a minimal volume of ethanol and stirred for 1 hour. Levorphanol tartrate dihydrate (0.4435 g, 1.00 mmol) is dissolved in a minimal volume of water and combined with the ethanolic ketoprofenate solution. The resulting solution is stirred and the total volume reduced to approximately 30 mL. The concentrated solution is transferred to a separatory funnel and the desired product extracted with diethyl ether (4×90 mL). The organic layers are combined and the solvent removed by rotary evaporation. The product is dried under vacuum overnight.

### EXAMPLE 125

### Preparation of Levorphanol Suprofenate from Levorphanol Tartrate Dihydrate and Suprofen

Levorphanol suprofenate may be prepared using the following synthetic scheme. Suprofen (0.2603 g, 1.00 mmol) dissolved in a minimal volume of ethanol is combined with potassium hydroxide (0.05611 g, 1.00 mmol) dissolved in a minimal volume of ethanol and stirred for 1 hour. Levorphanol tartrate dihydrate (0.4435 g, 1.00 mmol) is dissolved in a minimal volume of water and combined with the ethanolic suprofenate solution. The resulting solution is stirred and the total volume reduced to approximately 30 mL. The concentrated solution is transferred to a separatory funnel and the desired product extracted with diethyl ether (4×90 mL). The organic layers are combined and the solvent removed by rotary evaporation. The product is dried under vacuum overnight.

### EXAMPLE 126

### Preparation of Levorphanol (S)-Flurbiprofenate from Levorphanol Tartrate Dihydrate and (S)-Flurbiprofen

Levorphanol (S)-flurbiprofenate may be prepared using the following synthetic scheme. (S)-Flurbiprofen (0.2443 g, 1.00 mmol) dissolved in a minimal volume of ethanol is combined with potassium hydroxide (0.05611 g, 1.00 mmol) dissolved in a minimal volume of ethanol and stirred for 1 hour. Levorphanol tartrate dihydrate (0.4435 g, 1.00 mmol) is dissolved in a minimal volume of water and combined with the ethanolic flurbiprofenate solution. The resulting solution is stirred and the total volume reduced to approximately 30 mL. The concentrated solution is transferred to a separatory funnel and the desired product extracted with diethyl ether (4×90 mL). The organic layers are combined and the solvent removed by rotary evaporation. The product is dried under vacuum overnight.

### EXAMPLE 127

### Preparation of Levorphanol Tolmetinate from Levorphanol Tartrate Dihydrate and Tolmetin Sodium Dihydrate

Levorphanol tolmetinate may be prepared using the following synthetic scheme. Aqueous solutions of tolmetin sodium dihydrate (0.3153 g, 1.00 mmol) and of levorphanol tartrate dihydrate (0.4435 g, 1.00 mmol) are combined into a suitable flask and stirred for 60 minutes. The resulting solution is transferred to a separatory funnel and the desired product extracted with diethyl ether (4×90 mL). The organic layers are combined and the solvent removed by rotary evaporation. The product is dried under vacuum overnight.

### EXAMPLE 128

### Preparation of Levorphanol Fenoprofenate from Levorphanol Tartrate Dihydrate and Fenoprofen Calcium Trihydrate

Levorphanol fenoprofenate may be prepared using the following synthetic scheme. Aqueous solutions of fenoprofen calcium trihydrate (0.2884 g, 0.50 mmol) and of levorphanol tartrate dihydrate (0.4435 g, 1.00 mmol) are combined into a suitable flask and stirred for 60 minutes. The resulting solution is transferred to a separatory funnel and the desired product extracted with diethyl ether (4×90 mL). The organic layers are combined and the solvent removed by rotary evaporation. The product is dried under vacuum overnight.

### EXAMPLE 129

### Preparation of Levorphanol Oxaprozinate from Levorphanol Tartrate Dihydrate and Oxaprozin

Levorphanol oxaprozinate may be prepared using the following synthetic scheme. Oxaprozin (0.2933 g, 1.00 mmol) dissolved in a minimal volume of ethanol is combined with potassium hydroxide (0.05611 g, 1.00 mmol) dissolved in a minimal volume of ethanol and stirred for 1 hour. Levorphanol tartrate dihydrate (0.4435 g, 1.00 mmol) is dissolved in a minimal volume of water and combined with the ethanolic oxaprozinate solution. The resulting solution is stirred and the total volume reduced to approximately 30 mL. The concentrated solution is transferred to a separatory funnel and the desired product extracted with diethyl ether (4×90 mL). The organic layers are combined and the solvent removed by rotary evaporation. The product is dried under vacuum overnight.

### EXAMPLE 130

### Preparation of Levorphanol Difunisalate from Levorphanol Tartrate Dihydrate and Difunisal

Levorphanol difunisalate may be prepared using the following synthetic scheme. Difunisal (0.2502 g, 1.00 mmol) dissolved in a minimal volume of ethanol is combined with potassium hydroxide (0.05611 g, 1.00 mmol) dissolved in a minimal volume of ethanol and stirred for 1 hour. Levorphanol tartrate dihydrate (0.4435 g, 1.00 mmol) is dissolved in a minimal volume of water and combined with the ethanolic difunisalate solution. The resulting solution is stirred and the total volume reduced to approximately 30 mL. The concentrated solution is transferred to a separatory funnel and the desired product extracted with diethyl ether (4×90 mL). The organic layers are combined and the solvent removed by rotary evaporation. The product is dried under vacuum overnight.

### EXAMPLE 131

### Preparation of Levorphanol Loxoprofenate from Levorphanol Tartrate Dihydrate and Loxoprofen

Levorphanol loxoprofenate may be prepared using the following synthetic scheme. Loxoprofen (0.2463 g, 1.00 mmol) dissolved in a minimal volume of ethanol is combined with potassium hydroxide (0.05611 g, 1.00 mmol) dissolved in a minimal volume of ethanol and stirred for 1 hour. Levorphanol tartrate dihydrate (0.4435 g, 1.00 mmol) is dissolved in a minimal volume of water and combined with the ethanolic loxoprofenate solution. The resulting solution is stirred and the total volume is reduced to approximately 30 mL. The concentrated solution is transferred to a separatory funnel and the desired product extracted with diethyl ether (4×90 mL). The organic layers are combined and the solvent removed by rotary evaporation. The product is dried under vacuum overnight.

### EXAMPLE 132

### Preparation of Levorphanol Diclofenate from Levorphanol Tartrate Dihydrate and Sodium Diclofenac

Levorphanol diclofenate may be prepared using the following synthetic scheme. Aqueous solutions of sodium diclofenac (0.3181 g, 1.00 mmol) and of levorphanol tartrate dihydrate (0.4435 g, 1.00 mmol) are combined into a suitable flask and stirred for 60 minutes. The resulting solution is transferred to a separatory funnel and the desired product extracted with diethyl ether (4×90 mL). The organic layers are combined and the solvent removed by rotary evaporation. The product is dried under vacuum overnight.

### EXAMPLE 133

### Preparation of 5R,9R,13R,14S-Oxycodone Ibuprofenate from 5R,9R,13R,14S-Oxycodone Hydrochloride and Ibuprofen

5R,9R,13R,14S-Oxycodone ibuprofenate may be prepared using the following synthetic scheme. Ibuprofen (0.2063 g, 1.00 mmol) dissolved in a minimal volume of ethanol is combined with potassium hydroxide (0.05611 g, 1.00 mmol) dissolved in a minimal volume of ethanol and stirred for 1 hour. Oxycodone hydrochloride (0.3518 g, 1.00 mmol) is dissolved in a minimal volume of water and combined with the ethanolic ibuprofenate solution. The resulting solution is stirred and the total volume is reduced to approximately 30 mL. The concentrated solution is transferred to a separatory funnel and the desired product extracted with diethyl ether (4×90 mL). The organic layers are combined and the solvent removed by rotary evaporation. The product is dried under vacuum overnight.

### EXAMPLE 134

### Preparation of 5R,9R,13R,14S-Oxycodone Acetylsalicylate from 5R,9R,13R,14S-Oxycodone Hydrochloride and Acetylsalicylic Acid

R,9R,13R,14S-Oxycodone acetylsalicylate may be prepared using the following synthetic scheme. Acetylsalicylic acid (0.3003 g, 1.00 mmol) dissolved in a minimal volume of ethanol is combined with potassium hydroxide (0.05611 g, 1.00 mmol) dissolved in a minimal volume of ethanol and stirred for 1 hour. Oxycodone hydrochloride (0.3518 g, 1.00 mmol) is dissolved in a minimal volume of water and combined with the ethanolic acetylsalicylate solution. The resulting solution is stirred and the total volume reduced to approximately 30 mL. The concentrated solution is transferred to a separatory funnel and the desired product extracted with diethyl ether (4×90 mL). The organic layers are combined and the solvent removed by rotary evaporation. The product is dried under vacuum overnight.

### EXAMPLE 135

### Preparation of 5R,9R,13R,14S-Oxycodone Salicylate from 5R,9R,13R,14S-Oxycodone Hydrochloride and Sodium Salicylate

5R,9R,13R,14S-Oxycodone salicylate may be prepared using the following synthetic scheme. Aqueous solutions of sodium salicylate (0.1601 g, 1.00 mmol) and of oxycodone hydrochloride (0.3518 g, 1.00 mmol) are combined into a suitable flask and stirred for 60 minutes. The resulting solution is transferred to a separatory funnel and the desired product extracted with diethyl ether (4×90 mL). The organic layers are combined and the solvent removed by rotary evaporation. The product is dried under vacuum overnight.

### EXAMPLE 136

### Preparation of 5R,9R,13R,14S-Oxycodone Indomethacinate from 5R,9R,13R,14S-Oxycodone Hydrochloride and Indomethacin

5R,9R,13R,14S-Oxycodone indomethacinate may be prepared using the following synthetic scheme. Indomethacin (0.3578 g, 1.00 mmol) dissolved in a minimal volume of ethanol and combined with potassium hydroxide (0.05611 g, 1.00 mmol) dissolved in a minimal volume of ethanol and stirred for 1 hour. Oxycodone hydrochloride (0.3518 g, 1.00 mmol) is dissolved in a minimal volume of water and combined with the ethanolic indomethacinate solution. The resulting solution is stirred and the total volume is reduced to approximately 30 mL. The solution is transferred to a separatory funnel and the desired product extracted with diethyl ether (4×90 mL). The organic layers are combined and the solvent removed by rotary evaporation. The product is dried under vacuum overnight.

### EXAMPLE 137

### Preparation of 5R,9R,13R,14S-Oxycodone Naproxenate from 5R,9R,13R,14S-Oxycodone Hydrochloride and Naproxen Sodium

5R,9R,13R,14S-Oxycodone naproxenate may be prepared using the following synthetic scheme. Aqueous solutions of naproxen sodium (0.2522 g, 1.00 mmol) and of oxycodone hydrochloride (0.3518 g, 1.00 mmol) are combined into a suitable flask and stirred for 60 minutes. The resulting solution is transferred to a separatory funnel and the desired product extracted with diethyl ether (4×90 mL). The organic layers are combined and the solvent removed by rotary evaporation. The product is dried under vacuum overnight.

### EXAMPLE 137

### Preparation of 5R,9R,13R,14S-Oxycodone Etodolate from 5R,9R,13R,14S-Oxycodone Hydrochloride and Etodolac

5R,9R,13R,14S-Oxycodone etodolate may be prepared using the following synthetic scheme. Etodolac (0.2874 g, 1.00 mmol) dissolved in a minimal volume of ethanol is combined with potassium hydroxide (0.05611 g, 1.00 mmol) dissolved in a minimal volume of ethanol and stirred for 1 hour. Oxycodone hydrochloride (0.3518 g, 1.00 mmol) is dissolved in a minimal volume of water and combined with the ethanolic etodolate solution. The resulting solution is stirred and the total volume reduced to approximately 30 mL. The concentrated solution is transferred to a separatory funnel and the desired product extracted with diethyl ether (4×90 mL). The organic layers are combined and the solvent removed by rotary evaporation. The product is dried under vacuum overnight.

### EXAMPLE 138

### Preparation of 5R,9R,13R,14S-Oxycodone Sulindate from 5R,9R,13R,14S-Oxycodone Hydrochloride and Sulindac

5R,9R,13R,14S-Oxycodone sulindate may be prepared using the following synthetic scheme. Sulindac (0.3564 g, 1.00 mmol) dissolved in a minimal volume of ethanol is combined with potassium hydroxide (0.05611 g, 1.00 mmol) dissolved in a minimal volume of ethanol and stirred for 1 hour. Oxycodone hydrochloride (0.3518 g, 1.00 mol) is dissolved in a minimal volume of water and combined with the ethanolic sulindate solution. The resulting solution is stirred and the total volume reduced to approximately 30 mL. The concentrated solution is transferred to a separatory funnel and the desired product extracted with diethyl ether (4×90 mL). The organic layers are combined and the solvent removed by rotary evaporation. The product is dried under vacuum overnight.

### EXAMPLE 139

### Preparation of 5R,9R,13R,14S-Oxycodone (S)-Ketoprofenate from 5R,9R,13R,14S-Oxycodone Hydrochloride and (S)-Ketoprofen

5R,9R,13R,14S-Oxycodone (S)-ketoprofenate may be prepared using the following synthetic scheme. (S)-Ketoprofen (0.2543 g, 1.00 mmol) dissolved in a minimal volume of ethanol is combined with potassium hydroxide (0.05611 g, 1.00 mmol) dissolved in a minimal volume of ethanol and stirred for 1 hour. Oxycodone hydrochloride (0.3518 g, 1.00 mmol) is dissolved in a minimal volume of water and combined with the ethanolic ketoprofenate solution. The resulting solution is stirred and the total volume reduced to approximately 30 mL. The concentrated solution is transferred to a separatory funnel and the desired product extracted with diethyl ether (4×90 mL). The organic layers are combined and the solvent removed by rotary evaporation. The product is dried under vacuum overnight.

### EXAMPLE 140

### Preparation of 5R,9R,13R,14S-Oxycodone Suprofenate from 5R,9R,13R,14S-Oxycodone Hydrochloride and Suprofen

5R,9R,13R,14S-Oxycodone suprofenate may be prepared using the following synthetic scheme. Suprofen (0.2603 g, 1.00 mmol) dissolved in a minimal volume of ethanol is combined with potassium hydroxide (0.05611 g, 1.00 mmol) dissolved in a minimal volume of ethanol and stirred for 1 hour. Oxycodone hydrochloride (0.3518 g, 1.00 mol) is dissolved in a minimal volume of water and combined with the ethanolic suprofenate solution. The resulting solution is stirred and the total volume reduced to approximately 30 mL. The concentrated solution is transferred to a separatory funnel and the desired product extracted with diethyl ether (4×90 mL). The organic layers are combined and the solvent removed by rotary evaporation. The product is dried under vacuum overnight.

### EXAMPLE 141

### Preparation of 5R,9R,13R,14S-Oxycodone (S)-Flurbiprofenate from 5R,9R,13R,14S-Oxycodone Hydrochloride and (S)-Flurbiprofen

5R,9R,13R,14S-Oxycodone (S)-flurbiprofenate may be prepared using the following synthetic scheme. (S)-Flurbiprofen (0.2443 g, 1.00 mmol) dissolved in a minimal volume of ethanol is combined with potassium hydroxide (0.05611 g, 1.00 mmol) dissolved in a minimal volume of ethanol and stirred for 1 hour. Oxycodone hydrochloride (0.3518 g, 1.00 mmol) is dissolved in a minimal volume of water and combined with the ethanolic flurbiprofenate solution. The resulting solution is stirred and the total volume reduced to approximately 30 mL. The concentrated solution is transferred to a separatory funnel and the desired product extracted with diethyl ether (4×90 mL). The organic layers are combined and the solvent removed by rotary evaporation. The product is dried under vacuum overnight.

### EXAMPLE 142

### Preparation of 5R,9R,13R,14S-Oxycodone Tolmetinate from 5R,9R,13R,14S-Oxycodone Hydrochloride and Tolmetin Sodium Dihydrate

5R,9R,13R,14S-Oxycodone tolmetinate may be prepared using the following synthetic scheme. Aqueous solutions of tolmetin sodium dihydrate (0.3153 g, 1.00 mmol) and of oxycodone hydrochloride (0.3518 g, 1.00 mmol) are combined into a suitable flask and stirred for 60 minutes. The resulting solution is transferred to a separatory funnel and the desired product extracted with diethyl ether (4×90 mL). The organic layers are combined and the solvent removed by rotary evaporation. The product is dried under vacuum overnight.

### EXAMPLE 143

### Preparation of 5R,9R,13R,14S-Oxycodone Fenoprofenate from 5R,9R,13R,14S-Oxycodone Hydrochloride and Fenoprofen Calcium Trihydrate

5R,9R,13R,14S-Oxycodone fenoprofenate may be prepared using the following synthetic scheme. Aqueous solutions of fenoprofen calcium trihydrate (0.2884 g, 0.50 mmol) and of oxycodone hydrochloride (0.3518 g, 1.00 mmol) are combined into a suitable flask and stirred for 60 minutes. The resulting solution is transferred to a separatory funnel and the desired product extracted with diethyl ether (4×90 mL). The organic layers are combined and the solvent removed by rotary evaporation. The product is dried under vacuum overnight.

### EXAMPLE 144

### Preparation of 5R,9R,13R,14S-Oxycodone Oxaprozinate from 5R,9R,13R,14S-Oxycodone Hydrochloride and Oxaprozin

5R,9R,13R,14S-Oxycodone oxaprozinate may be prepared using the following synthetic scheme. Oxaprozin (0.2933 g, 1.00 mmol) dissolved in a minimal volume of ethanol is combined with potassium hydroxide (0.05611 g, 1.00 mmol) dissolved in a minimal volume of ethanol and stirred for 1 hour. Oxycodone hydrochloride (0.3518 g, 1.00 mmol) is dissolved in a minimal volume of water and combined with the ethanolic oxaprozinate solution. The resulting solution is stirred and the total volume reduced to approximately 30 mL. The concentrated solution is transferred to a separatory funnel and the desired product extracted with diethyl ether (4×90 mL). The organic layers are combined and the solvent removed by rotary evaporation. The product is dried under vacuum overnight.

### EXAMPLE 145

### Preparation of 5R,9R,13R,14S-Oxycodone Difunisalate from 5R,9R,13R,14S-Oxycodone Hydrochloride and Difunisal

5R,9R,13R,14S-Oxycodone difunisalate may be prepared using the following synthetic scheme. Difunisal (0.2502 g, 1.00 mmol) dissolved in a minimal volume of ethanol is combined with potassium hydroxide (0.05611 g, 1.00 mmol) dissolved in a minimal volume of ethanol and stirred for 1 hour. Oxycodone hydrochloride (0.3518 g, 1.00 mmol) is dissolved in a minimal volume of water and combined with the ethanolic difunisalate solution. The resulting solution is stirred and the total volume reduced to approximately 30 mL. The concentrated solution is transferred to a separatory funnel and the desired product extracted with diethyl ether (4×90 mL). The organic layers are combined and the solvent removed by rotary evaporation. The product is dried under vacuum overnight.

### EXAMPLE 146

### Preparation of 5R,9R,13R,14S-Oxycodone Loxoprofenate from 5R,9R,13R,14S-Oxycodone Hydrochloride and Loxoprofen

5R,9R,13R,14S-Oxycodone loxoprofenate may be prepared using the following synthetic scheme. Loxoprofen (0.2463 g, 1.00 mmol) is dissolved in a minimal volume of ethanol and combined with potassium hydroxide (0.05611 g, 1.00 mmol) dissolved in a minimal volume of ethanol and stirred for 1 hour. Oxycodone hydrochloride (0.3518 g, 1.00 mmol) is dissolved in a minimal volume of water and combined with the ethanolic loxoprofenate solution. The resulting solution is stirred and the total volume reduced to approximately 30 mL. The concentrated solution is transferred to a separatory funnel and the desired product extracted with diethyl ether (4×90 mL). The organic layers are combined and the solvent removed by rotary evaporation. The product is dried under vacuum overnight.

### EXAMPLE 147

### Particle Size Resulting When a Propoxyphene Diclofenate Solution is Added to Hydrochloric Acid

Particle size of a methanolic solution of propoxyphene diclofenate (61 mg/mL, 0.096 mmol/mL, 25 mL) was monitored for 2.5 hours by adding this solution to HCl (0.1 N, 25 mL). Measurements were obtained using a Sympatec HELOS model KF particle sizer with SUCELL and R5 lens (0.5-875 μm), pump, and stirrer speeds set to 50% of the maximum value. The SUCELL was filled with water and reference measurements were acquired before adding an appropriate amount of propoxyphene diclofenate solution for an approximate optical concentration of 10% at timepoints of 15, 45, 75, 120, and 150 minutes. The results are tabulated in Table 15.

TABLE 15
Results for Propoxyphene Diclofenate (61 mg/mL) in MeOH

| Timepoint | | | |
|---|---|---|---|
| (minutes) | Mean (μm) | ≦50% (μm) | ≦90% (μm) |
| 15 | 152.4 | 141.2 | 287.9 |
| 45 | 155.5 | 143.0 | 293.9 |
| 75 | 161.7 | 140.5 | 308.6 |
| 120 | 149.2 | 135.4 | 286.3 |
| 150 | 124.2 | 110.2 | 238.4 |

### EXAMPLE 148

Particle Size Resulting when a Propoxyphene Diclofenate Formulation is Added to Hydrochloric Acid

A propoxyphene diclofenate solution was prepared by adding propoxyphene diclofenate (12.5 mg, 0.020 mmol) to the contents of a placebo 50 mg capsule containing a dispersant and a solubilizer. The particle size of this solution was monitored for about 2 hours by adding the solution of propoxyphene diclofenate to Hcl (0.1 N, 25 mL). Measurements were obtained using a Sympatec HELOS model KF particle sizer with SUCELL and R5 lens (0.5-875 um), pump and stirrer speeds set to 50% of the maximum value. The SUCELL was filled with water and reference measurements were acquired before adding an appropriate amount of propoxyphene diclofenate solution for an approximate optical concentration of 10% at timepoints of 5, 10, 20, 30, 40, 60, 80, and 100 minutes. The data are shown in Table 16.

TABLE 16
Particle Size Results for Propoxyphene Diclofenate Formulation

| Timepoint | | | |
|---|---|---|---|
| (minutes) | Mean (μm) | ≦50% (μm) | ≦90% (μm) |
| 5 | 6.6 | 5.7 | 11.7 |
| 10 | 6.6 | 5.6 | 11.6 |
| 20 | 6.2 | 5.6 | 11.2 |
| 30 | 6.6 | 5.6 | 11.4 |
| 40 | 6.1 | 5.5 | 10.9 |
| 60 | 6.1 | 5.5 | 10.9 |
| 80 | 6.0 | 5.4 | 10.7 |
| 100 | 6.8 | 5.6 | 11.4 |

### EXAMPLE 49

### Particle Size Resulting When A Propoxyphene Diclofenate Formulation is Added to Hydrochloric Acid

A propoxyphene diclofenate solution was prepared by adding propoxyphene diclofenate (25 mg, 0.039 mmol) to the contents of a placebo 50 mg capsule containing a dispersant and a solubilizer. The particle size of this solution was monitored for about 2 hours by adding the solution of propoxyphene diclofenate to HCl (0.1 N, 25 mL). Measurements were obtained using a Sympatec HELOS model KF particle sizer with SUCELL and R5 lens (0.5-875 μm), pump, and stirrer speeds set to 50% of the maximum value. The SUCELL was filled with water and reference measurements were acquired before adding an appropriate amount of Propoxyphene Diclofenate solution for an approximate optical concentration of 10% at timepoints of 5, 10, 20, 30, 40, 60, 80, and 100 minutes. The data are shown in Table 17.

TABLE 17
Particle Size Results for Propoxyphene Diclofenate Formulation

| Timepoint | | | |
|---|---|---|---|
| (minutes) | Mean (μm) | ≦50% (μm) | ≦90% (μm) |
| 5 | 8.8 | 6.9 | 17.5 |
| 10 | 8.0 | 6.5 | 15.1 |
| 20 | 7.4 | 6.1 | 13.8 |
| 30 | 7.5 | 6.0 | 13.5 |
| 40 | 7.1 | 5.9 | 12.9 |
| 60 | 6.9 | 5.8 | 12.4 |
| 80 | 7.1 | 5.7 | 12.3 |
| 100 | 6.8 | 5.7 | 12.1 |

### EXAMPLE 150

### Particle Size Resulting when a Propoxyphene Diclofenate Formulation is Added to Hydrochloric Acid

A propoxyphene diclofenate solution was prepared by adding propoxyphene diclofenate (40 mg, 0.063 mmol) to the contents of a placebo 50 mg capsule containing a dispersant and a solubilizer. The particle size of this solution was monitored for about 2 hours by adding the solution of propoxyphene diclofenate to HCl (0.1 N, 25 mL). Measurements were obtained using a Sympatec HELOS model KF particle sizer with SUCELL and R5 lens (0.5-875 mm), pump, and stirrer speeds set to 50% of the maximum value. The SUCELL was filled with water and reference measurements were acquired before adding an appropriate amount of propoxyphene diclofenate solution for an approximate optical concentration of 10% at timepoints of 20, 40, 60, 90, and 120 minutes. The data are shown in Table 18.

TABLE 18
Particle Size Results for Propoxyphene Diclofenate Formulation

| Timepoint | | | |
|---|---|---|---|
| (minutes) | Mean (μm) | ≦50% (μm) | ≦90% (μm) |
| 20 | 9.1 | 7.0 | 18.0 |
| 40 | 8.8 | 6.7 | 17.3 |
| 60 | 8.5 | 6.4 | 16.0 |
| 90 | 7.9 | 6.1 | 14.8 |
| 120 | 7.8 | 6.0 | 14.4 |

### EXAMPLE 151

### Particle Size Resulting when a Propoxyphene Diclofenate Formulation is Added to Hydrochloric Acid

A propoxyphene diclofenate solution was prepared by adding propoxyphene diclofenate (50 mg, 0.079 mmol) to the contents of a placebo 50 mg capsule containing a dispersant and a solubilizer. The particle size of this solution was monitored for about 2 hours by adding the solution of propoxyphene diclofenate to HCl (0.1 N, 25 mL). Measurements were obtained using a Sympatec HELOS model KF particle sizer with SUCELL and R5 lens (0.5-875 mm), pump, and stirrer speeds set to 50% of the maximum value. The SUCELL was filled with water and reference measurements were acquired before adding an appropriate amount of propoxyphene diclofenate solution for an approximate optical concentration of 10% at timepoints of 5, 10, 20, 30, 40, 60, 80, and 120 minutes. The data are shown in Table 19.

TABLE 19
Particle Size Results for Propoxyphene Diclofenate Formulation

| Timepoint | | | |
|---|---|---|---|
| (minutes) | Mean (μm) | ≦50% (μm) | ≦90% (μm) |
| 5 | 9.1 | 7.0 | 17.9 |
| 10 | 9.4 | 6.9 | 18.4 |
| 20 | 9.1 | 6.6 | 17.4 |
| 30 | 8.6 | 6.3 | 16.1 |
| 40 | 8.3 | 6.1 | 15.4 |
| 60 | 8.5 | 5.9 | 15.1 |
| 80 | 8.0 | 5.8 | 14.3 |
| 120 | 7.8 | 5.7 | 14.2 |

### EXAMPLE 152

### Solubility when Propoxyphene Diclofenate is Added to Water at Various pH Values

Propoxyphene diclofenate solutions were prepared by adding propoxyphene diclofenate (approx. 48 mg, 0.076 mmol) to dissolution vessels containing water (400 mL) at pH of 2, 3, 5, 7, 9 and 11 and equilibrated at 36.8° C. The solutions were stirred by paddles at 150 RPM for approximately 12 hours. Final sample solutions were prepared by diluting 12.5 mL of the propoxyphene diclofenate solution from each vessel filtered through a 0.45 μm Nylon filter to 50.0 mL with methanol. Five standard solutions of propoxyphene diclofenate were prepared at concentrations ranging from 0.00091 to 0.02914 mg/mL. The standards and sample preparations were measured at 282 nm in a 1 cm cell using a UV spectrophotometer. The results were determined from the linearity curve generated from the standard data. The data are shown in Table 20.

TABLE 20
Solubility Results for Propoxyphene
Diclofenate as a Function of pH

| | mg Dissolved from | | |
|---|---|---|---|
| pH | 48 mg Solution | mg/mL Dissolved | % Dissolved |
| 3 | 0.004 | 1.0528 × 10⁻⁵ | 0.009 |
| 5 | 36.005 | 0.09 | 74.878 |
| 7 | 36.644 | 0.0916 | 75.677 |
| 9 | 34.018 | 0.08504 | 71.168 |
| 11 | 18.700 | 0.04676 | 39.395 |

### EXAMPLE 153

### Solubility of Propoxyphene Diclofenate in Polyethylene Glycol

The total solubility of propoxyphene diclofenate in polyethylene glycol 400 was determined to exceed 670 mg/mL. The solubility was determined by UV detection using a standard solution (0.049 mmol/L).

### EXAMPLE 154

### Preparation of (2S,3R)-(+)-4-(Dimethylamino)-3-methyl-1,2-diphenyl-2-butanol Propionate Diclofenate from Sodium Diclofenac and (2S,3R)-(+)-4-(Dimethylamino)-3-methyl-1,2-diphenyl-2-butanol Propionate (Propoxyphene) Hydrochloride

Sodium diclofenac (133.7 g, 0.4203 mol) was dissolved in water (2500 mL) at about 50° C. with mechanical stirring. To this a 50° C. solution of (2S,3R)-(+)-4-(dimethylamino)-3-methyl-1,2-diphenyl-2-butanol propionate hydrochloride (158.6 g, 0.4219 mol) in water (600 mL) was slowly added while vigorously stirring the mixture with a mechanical stirrer and maintaining the temperature at about 50° C. A thick sticky white precipitate formed as the mixture was stirred. The reaction was monitored by HPLC to calculate the amount of propoxyphene remaining in solution. When the reaction was considered complete, as evidence by the disappearance of propoxyphene from solution, the solution was decanted, and the solid product washed with multiple aliquots of water (about 2000 mL) at 50° C. with mechanical stirring until HPLC confirmed only low levels of unreacted sodium diclofenac present. The solid material was then dissolved in a minimal amount of acetone and the acetone subsequently removed by rotary evaporation under vacuum to yield a white solid. The white solid was then removed from the flask and spread over the bottom of a crystallizing dish which is placed in a vacuum oven for prolonged drying at 30° C. to remove any odor of residual acetone. Yield of the title compound was 239.2 g (0.3763 mol; 89.5%).

### EXAMPLE 155

### Preparation of (2S,3R)-(+)-4-(Dimethylamino)-3-methyl-1,2-diphenyl-2-butanol Propionate Diclofenate from Sodium Diclofenac and (2S,3R)-(+)-4-(Dimethylamino)-3-methyl-1,2-diphenyl-2-butanol Propionate (Propoxyphene) Hydrochloride

Propoxyphene hydrochloride (117.0 g, 0.3112 mol) was dissolved in water (1500 mL) at about 50° C. with mechanical stirring. To this a 50° C. solution of sodium diclofenac (108.2 g, 0.34 mol) in water (2000 mL) was slowly added while vigorously stirring the mixture with a mechanical stirrer and maintaining the temperature at about 50° C. A thick sticky white precipitate formed as the solution was stirred. Completeness of reaction was confirmed by HPLC to determine the amount of unreacted propoxyphene hydrochloride remaining in solution (about 1 mg/mL remained). The reaction was considered complete, the solution was decanted, and the solid product washed with numerous aliquots of water (about 300 mL for each washing) at about 50° C. with mechanical stirring until HPLC confirmed only low levels of unreacted sodium diclofenate remained (about 0.2 mg/mL). The solid material was then dissolved in a minimal amount of acetone and the acetone subsequently removed by rotary evaporation to yield a white solid. The white solid was then removed from the flask and spread over the bottom of a crystallizing dish which was placed in a vacuum oven for prolonged drying at 30° C. to remove residual acetone. Acetone removal was considered complete when no odor of residual acetone remained. Yield of the title compound was 150.9 g (0.2374 mol; 76.3%).

### EXAMPLE 156

### Preparation of (2S,3R)-(+)-4-(Dimethylamino)-3-methyl-1,2-diphenyl-2-butanol Propionate Diclofenate from Potassium Diclofenac and (2S,3R)-(+)-4-(Dimethylamino)-3-methyl-1,2-diphenyl-2-butanol Propionate (Propoxyphene) Hydrochloride

Potassium diclofenac (335.2 g, 1.003 mol) was dissolved in water (2000 mL) at about 50° C. with mechanical stirring. To this a 50° C. solution of (2S,3R)-(+)-4-(dimethylamino)-3-methyl-1,2-diphenyl-2-butanol propionate hydrochloride (376.6 g, 1.002 mol) in water (700 mL) was slowly added while vigorously stirring the mixture with a mechanical stirrer and maintaining the temperature at about 50° C. A thick sticky white precipitate formed as the solution was stirred over several hours. Completeness of reaction was confirmed by HPLC to determine the amount of unreacted propoxyphene hydrochloride remaining in solution (about 1 mg/mL remained). The reaction was considered complete when about 1 mg/mL propoxyphene hydrochloride remained in solution. The solvent was decanted, and the solid product washed with numerous aliquots of water (about 500 mL for each washing) at about 50° C. with mechanical stirring until HPLC confirmed only low levels of unreacted sodium diclofenate remained (about 0.2 mg/mL). The solid material was then dissolved in a minimal amount of acetone and the acetone subsequently removed by rotary evaporation to yield a white solid. The white solid was then removed from the flask and spread over the bottom of a crystallizing dish which was placed in a vacuum oven for prolonged drying at 30° C. to remove residual acetone. Acetone removal was considered complete when no odor of residual acetone remained. Yield of the title compound was 613.2 g (0.9647 mol; 96.3%).

### EXAMPLE 157

### Analysis of Washings of Propoxyphene Diclofenate Synthesis Using High Pressure Liquid Chromatography (HPLC)

During the synthesis of propoxyphene diclofenate in the foregoing examples, the product was washed with water to remove excess diclofenac (sodium or potassium). The levels of diclofenac salts were monitored to determine the reaction end point by HPLC according to the following procedure.

HPLC was performed with the HP1100 system (Hewlett Packard, Palo Alto, Calif.). The method utilized a 4.6×150 mm C₁₈ column (Waters Corporation, Milford, Mass.) maintained at room temperature. The mobile phase was gradient controlled, consisting of Mobile Phase A (MP_{A}), a 90:10 mixture of water (4 drops trifluoroacetic acid (TFA) per 900 mL); and Mobile Phase B (MP_{B}), a 70:30 mixture of acetonitril:water. The gradient program was set as follows:

| Time (min) | % MP_{A} | % MP_{B} |
|---|---|---|
| 0.0 | 95.0 | 95.0 |
| 30.0 | 5.0 | 95.0 |
| 31.0 | 5.0 | 95.0 |
| 32.0 | 95.0 | 5.0 |

The flow rate was maintained at 1.0 mL/minute. Standards and sample solutions were prepared in water at concentrations of 0.3, 0.6, and 1.0 mM. Injection volume for sample and standard preparations (diclofenac potassium (Yung Zip); propoxyphene HCl (Mallinckrodt)) was 10 μL and run time was about 32 minutes. UV detection was performed at 217 nm. The chromatographic data peak areas were collected and analyzed using Millenium³² chromatography software (Waters Corporation, Milford, Mass.) to generate the % w/w assay values for the samples.

### EXAMPLE 158

### Analysis of the Aqueous Mother Liquor and Subsequent Washings During Propoxyphene Diclofenate Synthesis Using High Pressure Liquid Chromatography

During the synthesis of propoxyphene diclofenate as described in the foregoing examples the reaction was considered complete once the reaction mixture contained an acceptably low level of propoxyphene as determined by HPLC. When the reaction was complete, the aqueous mother liquor was decanted and the product washed with numerous aqueous rinses to remove excess diclofenac (sodium or potassium). Using an HPLC analysis according to the following procedure, the propoxyphene and diclofenac levels were monitored during the reaction to determine the end point of the reaction, and the point when sufficient washing had been accomplished.

For each of the procedures above, HPLC was performed with the HP 1100 system (Hewlett Packard, Palo Alto, Calif.). The method utilized a 4.6×150 mm C₁₈ column (Waters Corporation, Milford, Mass.) maintained at room temperature. The mobile phase was gradient controlled, consisting of Mobile Phase A; a 90:10 mixture of water (4 drops trifluoroacetic acid (TFA) per 900 mL):acetonitrile, and Mobile Phase B; a 70:30 mixture of acetonitrile:water. The gradient program was set as follows:

| Time (min) | % MP_{A} | % MP_{B} |
|---|---|---|
| 0.0 | 95.0 | 95.0 |
| 30.0 | 5.0 | 95.0 |
| 31.0 | 5.0 | 95.0 |
| 32.0 | 95.0 | 5.0 |

The flow rate was maintained at 1.0 mL/minute. Standard and sample solutions were prepared in water at concentrations of 0.3, 0.6, and 1.0 mM. Injection volume for the sample and standard preparations (diclofenac potassium (Yung Zip); propoxyphene HCl (Mallinckrodt)) was 10 μL and runtime of the analysis was about 32 minutes. UV detection was performed at 217 nm. The chromatographic data peak areas were collected and analyzed using Millennium³² chromatography software (Waters Corporation, Milford, Mass.) to generate the % w/w values for the samples.

1. An ion pair compound according to general formula I:
[narcotic]⁺[A]⁻ (I)
wherein
[narcotic]⁺ represents at least one cation of at least one narcotic agent or one or more stereochemical isomers thereof, and
[A]⁻ represents an anion of at least one NSAID or one or more stereochemical isomers thereof,
or a pharmaceutically acceptable solvate, hydrate, polymorph, isotopically labeled version, or combination thereof.

2. The ion pair compound according to claim 1, wherein the narcotic in [narcotic]⁺ is selected from the group consisting of ketamine, oxycodone, propoxyphene, methadone, hydrocodone, morphine, codeine, fentanyl, meperidine, hydromorphone, oxymorphone, dihydrocodeine, nalbuphine, and buprenorphine.

3. The ion pair compound according to claim 2, wherein the narcotic in [narcotic]⁺ is selected from the group consisting of ketamine, oxycodone, propoxyphene, methadone, hydrocodone, morphine, and codeine.

4. The ion pair compound according to claim 3, wherein the narcotic is propoxyphene.

5. The ion pair compound according to claim 3, wherein the narcotic is codeine.

6. The ion pair compound according to claim 3, wherein the narcotic is hydrocodone.

7. The ion pair compound according to claim 3, wherein the narcotic is oxycodone.

8. The ion pair compound according to claim 3, wherein the narcotic is morphine.

9. The ion pair compound according to claim 3, wherein the narcotic is methadone.

10. The ion pair compound according to claim 1, wherein A in [A]⁻ is selected from the group consisting of nonselective COX inhibitors, selective COX-2 inhibitors, COX-LOX inhibitors, PLA₂ inhibitors, and combinations thereof.

11. The ion pair compound according to claim 10, wherein said NSAID is a nonselective COX inhibitor.

12. The ion pair compound according to claim 10, wherein said NSAID is a selective COX-2 inhibitor.

13. The ion pair compound according to claim 10, wherein said NSAID is a COX-LOX inhibitor.

14. The ion pair compound according to claim 10, wherein said NSAID is a PLA₂ inhibitor.

15. The ion pair compound according to claim 2, wherein A in [A]⁻ is selected from the group consisting of nonselective COX inhibitors, selective COX-2 inhibitors, COX-LOX inhibitors, PLA₂ inhibitors, and combinations thereof.

16. The ion pair compound according to claim 15, wherein said NSAID is a nonselective COX inhibitor.

17. The ion pair compound according to claim 15, wherein said NSAID is a selective COX-2 inhibitor.

18. The ion pair compound according to claim 15, wherein said NSAID is a COX-LOX inhibitor.

19. The ion pair compound according to claim 15, wherein said NSAID is a PLA₂ inhibitor.

20. The ion pair compound according to claim 1, wherein the NSAID is selected from the group consisting of diclofenac, etodolac, sulindac, alclofenac, fenclofenac, diflunisal, benorylate, fosfosal, salicylic acid, acetylsalicylic acid, ibuprofen, ketoprofen, naproxen, carprofen, fenbufen, flurbiprofen, oxaprozin, suprofen, triaprofenic acid, fenoprofen, indoprofen, piroprofen, flufenamic, mefenamic, meclofenamic, niflumic, salsalate, rolmerin, fentiazac, tilomisole, oxyphenbutazone, phenylbutazone, apazone, feprazone, sudoxicam, isoxicam, tenoxicam, piroxicam, indomethacin, meloxicam, nabumetone, tolmetin, lumiracoxib, and parecoxib.

21. The ion pair compound according to claim 20, wherein the NSAID is selected from the group consisting of diclofenac, etodolac, sulindac, salicylic acid, acetylsalicylic acid, ibuprofen, ketoprofen, naproxen, lumiracoxib, and parecoxib.

22. The ion pair compound according to claim 20, wherein the NSAID is diclofenac.

23. The ion pair compound according to claim 20, wherein the NSAID is lumiracoxib.

24. The ion pair compound according to claim 2, wherein the NSAID is selected from the group consisting of diclofenac, etodolac, sulindac, alclofenac, fenclofenac, diflunisal, benorylate, fosfosal, salicylic acid, acetylsalicylic acid, ibuprofen, ketoprofen, naproxen, carprofen, fenbufen, flurbiprofen, oxaprozin, suprofen, triaprofenic acid, fenoprofen, indoprofen, piroprofen, flufenamic, mefenamic, meclofenamic, niflumic, salsalate, rolmerin, fentiazac, tilomisole, oxyphenbutazone, phenylbutazone, apazone, feprazone, sudoxicam, isoxicam, tenoxicam, piroxicam, indomethacin, meloxicam, nabumetone, tolmetin, lumiracoxib, and parecoxib.

25. The ion pair compound according to claim 24, wherein the NSAID is selected from the group consisting of diclofenac, etodolac, sulindac, salicylic acid, acetylsalicylic acid, ibuprofen, ketoprofen, naproxen, lumiracoxib, and parecoxib.

26. The ion pair compound according to claim 1, wherein the compound is selected from the group consisting of propoxyphene diclofenate, ketamine diclofenate, methadone diclofenate, hydrocodone diclofenate, codeine diclofenate, propoxyphene salicylate, propoxyphene acetylsalicylate, propoxyphene ibuprofenate, morphine diclofenate, and oxycodone diclofenate.

27. The ion pair compound according to claim 24, wherein the compound is selected from the group consisting of propoxyphene diclofenate, ketamine diclofenate, methadone diclofenate, hydrocodone diclofenate, codeine diclofenate, morphine diclofenate, and oxycodone diclofenate.

28. The ion pair compound according to claim 24, wherein the compound is selected from the group consisting of propoxyphene salicylate, propoxyphene acetylsalicylate, and propoxyphene ibuprofenate.

29. The ion pair compound according to claim 1, wherein the compound is selected from the group consisting of propoxyphene lumiracoxibate, ketamine lumiracoxibate, methadone lumiracoxibate, hydrocodone lumiracoxibate, codeine lumiracoxibate, morphine lumiracoxibate, and oxycodone lumiracoxibate.

30. The ion pair compound according to claim 1, wherein the compound is selected from the group consisting of propoxyphene parecoxibate, ketamine parecoxibate, methadone parecoxibate, hydrocodone parecoxibate, codeine parecoxibate, morphine parecoxibate, and oxycodone parecoxibate.

31. The ion pair compound according to claim 1, wherein the compound is selected from the group consisting of propoxyphene naproxenate, propoxyphene etodolate, propoxyphene ketoprofenate, propoxyphene sulindate, propoxyphene suprofenate, propoxyphene flurbiprofenate, propoxyphene tolmetinate, propoxyphene fenoprofenate, propoxyphene oxaprozinate, propoxyphene difunisalate, propoxyphene loxoprofenate,
ketamine ibuprofenate, ketamine acetylsalicylate, ketamine indomethacinate, ketamine naproxenate, ketamine etodolate, ketamine sulindate, ketamine ketoprofenate, ketamine suprofenate, ketamine flurbiprofenate, ketamine tolmetinate, ketamine fenoprofenate, ketamine oxaprozinate, ketamine difunisalate, ketamine loxoprofenate, ketamine salicylate, ketamine diclofenate,
methadone ibuprofenate, methadone acetylsalicylate, methadone salicylate, methadone indomethacinate, methadone naproxenate, methadone etodolate, methadone sulinate, methadone ketoprofenate, methadone suprofenate, methadone flurbiprofenate, methadone tolmetinate, methadone fenoprofenate, methadone oxaprozinate, methadone difunisalate, methadone loxoprofenate,
hydrocodone ibuprofenate, hydrocodone acetylsalicylate, hydrocodone salicylate, hydrocodone indomethacinate, hydrocodone naproxenate, hydrocodone etodolate, hydrocodone sulindate, hydrocodone ketoprofenate, hydrocodone suprofenate, hydrocodone flurbiprofenate, hydrocodone tolmetinate, hydrocodone fenoprofenate, hydrocodone oxaprozinate, hydrocodone difunisalate, hydrocodone loxoprofenate,
codeine ibuprofenate, codeine acetylsalicylate, codeine salicylate, codeine indomethacinate, codeine naproxenate, codeine etodolate, codeine sulindate, codeine ketoprofenate, codeine suprofenate, codeine flurbiprofenate, codeine tolmetinate, codeine fenoprofenate, codeine oxaprozinate, codeine difunisalate, codeine loxoprofenate,
morphine ibuprofenate, morphine acetylsalicylate, morphine salicylate, morphine indomethacinate, morphine naproxenate, morphine etodolate, morphine sulindate, morphine ketoprofenate, morphine suprofenate, morphine flurbiprofenate, morphine tolmetinate, morphine fenoprofenate, morphine oxaprozinate, morphine difunisalate, morphine loxoprofenate,
levorphanol ibuprofenate, levorphanol acetylsalicylate, levorphanol salicylate, levorphanol indomethacinate, levorphanol naproxenate, levorphanol etodolate, levorphanol sulindate, levorphanol ketoprofenate, levorphanol suprofenate, levorphanol flurbiprofenate, levorphanol tolmetinate, levorphanol fenoprofenate, levorphanol oxaprozinate, levorphanol difunisalate, levorphanol loxoprofenate,
oxycodone ibuprofenate, oxycodone acetylsalicylate, oxycodone salicylate, oxycodone indomethacinate, oxycodone naproxenate, oxycodone etodolate, oxycodone sulindate, oxycodone ketoprofenate, oxycodone suprofenate, oxycodone flurbiprofenate, oxycodone tolmetinate, oxycodone fenoprofenate, oxycodone oxaprozinate, oxycodone difunisalate, oxycodone loxoprofenate,
fentanyl naproxenate, fentanyl etodolate, fentanyl ketoprofenate, fentanyl sulindate, fentanyl suprofenate, fentanyl flurbiprofenate, fentanyl tolmetinate, fentanyl fenoprofenate, fentanyl oxaprozinate, fentanyl difinisalate, fentanyl loxoprofenate,
meperidine naproxenate, meperidine etodolate, meperidine ketoprofenate, meperidine sulindate, meperidine suprofenate, meperidine flurbiprofenate, meperidine tolmetinate, meperidine fenoprofenate, meperidine oxaprozinate, meperidine difunisalate, meperidine loxoprofenate,
hydromorphone naproxenate, hydromorphone etodolate, hydromorphone ketoprofenate, hydromorphone sulindate, hydromorphone suprofenate, hydromorphone flurbiprofenate, hydromorphone tolmetinate, hydromorphone fenoprofenate, hydromorphone oxaprozinate, hydromorphone difunisalate, hydromorphone loxoprofenate,
oxymorphone naproxenate, oxymorphone etodolate, oxymorphone ketoprofenate, oxymorphone sulindate, oxymorphone suprofenate, oxymorphone flurbiprofenate, oxymorphone tolmetinate, oxymorphone fenoprofenate, oxymorphone oxaprozinate, oxymorphone difunisalate, oxymorphone loxoprofenate,
dihydrocodeine naproxenate, dihydrocodeine etodolate, dihydrocodeine ketoprofenate, dihydrocodeine sulindate, dihydrocodeine suprofenate, dihydrocodeine flurbiprofenate, dihydrocodeine tolmetinate, dihydrocodeine fenoprofenate, dihydrocodeine oxaprozinate, dihydrocodeine difunisalate, and dihydrocodeine loxoprofenate.

32. The ion pair compound according to claim 1, wherein the compound is propoxyphene diclofenate.

33. A pharmaceutical composition comprising a therapeutically effective amount of the ion pair compound according to claim 1 and a pharmaceutically acceptable carrier, diluent, excipient, stimulant, or combination thereof.

34. The pharmaceutical composition according to claim 33, wherein the composition further comprises a therapeutically effective amount of an additional NSAID.

35. The pharmaceutical composition according to claim 34, wherein the additional NSAID is the same as A, or a pharmaceutically acceptable salt, solvate, hydrate, polymorph, isotopically labeled version, or combination thereof.

36. The pharmaceutical composition according to claim 35, wherein the ion pair compound is propoxyphene diclofenate.

37. The pharmaceutical composition according to claim 36, wherein the additional NSAID is selected from the group consisting of diclofenac free acid and pharmaceutically acceptable salts thereof.

38. The pharmaceutical composition according to claim 37, wherein the pharmaceutically acceptable salts are selected from sodium and potassium salts.

39. The pharmaceutical composition according to claim 34, wherein the additional NSAID is different from A.

40. The pharmaceutical composition according to claim 33, wherein the composition further comprises a therapeutically effective amount of an additional narcotic,

41. The pharmaceutical composition according to claim 40, wherein the additional narcotic is the same as the narcotic in [narcotic]⁺, or a pharmaceutically acceptable salt, solvate, hydrate, polymorph, isotopically labeled version, or combination thereof.

42. The pharmaceutical composition according to claim 41, wherein the additional narcotic is different from the narcotic in [narcotic]⁺.

43. The pharmaceutical composition according to claim 33, wherein the ion pair compound is selected from the group consisting of propoxyphene diclofenate, ketamine diclofenate, methadone diclofenate, hydrocodone diclofenate, codeine diclofenate, propoxyphene salicylate, propoxyphene acetylsalicylate propoxyphene ibuprofenate, morphine diclofenate, and oxycodone diclofenate.

44. The pharmaceutical composition according to claim 33, wherein the compound is selected from the group consisting of propoxyphene naproxenate, propoxyphene etodolate, propoxyphene ketoprofenate, propoxyphene sulindate, propoxyphene suprofenate, propoxyphene flurbiprofenate, propoxyphene tolmetinate, propoxyphene fenoprofenate, propoxyphene oxaprozinate, propoxyphene difunisalate, propoxyphene loxoprofenate,
ketamine ibuprofenate, ketamine acetylsalicylate, ketamine indomethacinate, ketamine naproxenate, ketamine etodolate, ketamine sulindate, ketamine ketoprofenate, ketamine suprofenate, ketamine flurbiprofenate, ketamine tolmetinate, ketamine fenoprofenate, ketamine oxaprozinate, ketamine difunisalate, ketamine loxoprofenate, ketamine salicylate, ketamine diclofenate,
methadone ibuprofenate, methadone acetylsalicylate, methadone salicylate, methadone indomethacinate, methadone naproxenate, methadone etodolate, methadone sulinate, methadone ketoprofenate, methadone suprofenate, methadone flurbiprofenate, methadone tolmetinate, methadone fenoprofenate, methadone oxaprozinate, methadone difunisalate, methadone loxoprofenate,
hydrocodone ibuprofenate, hydrocodone acetylsalicylate, hydrocodone salicylate, hydrocodone indomethacinate, hydrocodone naproxenate, hydrocodone etodolate, hydrocodone sulindate, hydrocodone ketoprofenate, hydrocodone suprofenate, hydrocodone flurbiprofenate, hydrocodone tolmetinate, hydrocodone fenoprofenate, hydrocodone oxaprozinate, hydrocodone difunisalate, hydrocodone loxoprofenate,
codeine ibuprofenate, codeine acetylsalicylate, codeine salicylate, codeine indomethacinate, codeine naproxenate, codeine etodolate, codeine sulindate, codeine ketoprofenate, codeine suprofenate, codeine flurbiprofenate, codeine tolmetinate, codeine fenoprofenate, codeine oxaprozinate, codeine difunisalate, codeine loxoprofenate,
morphine ibuprofenate, morphine acetylsalicylate, morphine salicylate, morphine indomethacinate, morphine naproxenate, morphine etodolate, morphine sulindate, morphine ketoprofenate, morphine suprofenate, morphine flurbiprofenate, morphine tolmetinate, morphine fenoprofenate, morphine oxaprozinate, morphine difunisalate, morphine loxoprofenate,
levorphanol ibuprofenate, levorphanol acetylsalicylate, levorphanol salicylate, levorphanol indomethacinate, levorphanol naproxenate, levorphanol etodolate, levorphanol sulindate, levorphanol ketoprofenate, levorphanol suprofenate, levorphanol flurbiprofenate, levorphanol tolmetinate, levorphanol fenoprofenate, levorphanol oxaprozinate, levorphanol difunisalate, levorphanol loxoprofenate,
oxycodone ibuprofenate, oxycodone acetylsalicylate, oxycodone salicylate, oxycodone indomethacinate, oxycodone naproxenate, oxycodone etodolate, oxycodone sulindate, oxycodone ketoprofenate, oxycodone suprofenate, oxycodone flurbiprofenate, oxycodone tolmetinate, oxycodone fenoprofenate, oxycodone oxaprozinate, oxycodone difunisalate, oxycodone loxoprofenate,
fentanyl naproxenate, fentanyl etodolate, fentanyl ketoprofenate, fentanyl sulindate, fentanyl suprofenate, fentanyl flurbiprofenate, fentanyl tolmetinate, fentanyl fenoprofenate, fentanyl oxaprozinate, fentanyl difunisalate, fentanyl loxoprofenate,
meperidine naproxenate, meperidine etodolate, meperidine ketoprofenate, meperidine sulindate, meperidine suprofenate, meperidine flurbiprofenate, meperidine tolmetinate, meperidine fenoprofenate, meperidine oxaprozinate, meperidine difunisalate, meperidine loxoprofenate,
hydromorphone naproxenate, hydromorphone etodolate, hydromorphone ketoprofenate, hydromorphone sulindate, hydromorphone suprofenate, hydromorphone flurbiprofenate, hydromorphone tolmetinate, hydromorphone fenoprofenate, hydromorphone oxaprozinate, hydromorphone difunisalate, hydromorphone loxoprofenate,
oxymorphone naproxenate, oxymorphone etodolate, oxymorphone ketoprofenate, oxymorphone sulindate, oxymorphone suprofenate, oxymorphone flurbiprofenate, oxymorphone tolmetinate, oxymorphone fenoprofenate, oxymorphone oxaprozinate, oxymorphone difunisalate, oxymorphone loxoprofenate,
dihydrocodeine naproxenate, dihydrocodeine etodolate, dihydrocodeine ketoprofenate, dihydrocodeine sulindate, dihydrocodeine suprofenate, dihydrocodeine flurbiprofenate, dihydrocodeine tolmetinate, dihydrocodeine fenoprofenate, dihydrocodeine oxaprozinate, dihydrocodeine difunisalate, and dihydrocodeine loxoprofenate.

45. The pharmaceutical composition according to claim 40, wherein the ion pair compound is propoxyphene diclofenate.

46. The pharmaceutical composition according to claim 33, further comprising:
at least one dispersing agent selected from the group consisting of polymer-based dispersing agents and carbohydrate-based dispersing agents,
wherein the ratio of the ion pair compound to the polymer-based dispersing agent is from about 3:1 (w/w) to about 1:50 (w/w), and
wherein the ratio of the ion pair compound to the carbohydrate-based dispersing agent is from about 3:1 (w/w) to about 1:20 (w/w); and
at least one solubilizing agent; and, optionally,
at least one surfactant; and, further optionally,
at least one plasticizing agent.

47. The pharmaceutical composition according to claim 46, wherein at least one dispersing agent is a polymer-based dispersing agent.

48. The pharmaceutical composition according to claim 47, wherein the polymer-based dispersing agent is polyvinylpyrrolidone.

49. The pharmaceutical composition according to claim 48, wherein the polyvinylpyrrolidone is polyvinylpyrrolidone K29-32.

50. The pharmaceutical composition according to claim 46, wherein at least one dispersing agent is a carbohydrate-based dispersing agent.

51. The pharmaceutical composition according to claim 50, wherein the carbohydrate-based dispersing agent is selected from the group consisting of hydroxypropylcellulose, hydroxymethylcellulose, and cyclodextrin.

52. The pharmaceutical composition according to claim 46, wherein the composition comprises a solubilizing agent.

53. The pharmaceutical composition according to claim 52, wherein the solubilizing agent is selected from the group consisting of water and polyethylene glycol.

54. The pharmaceutical composition according to claim 53, wherein the molecular weight of the polyethylene glycol is from about 200 g/mol to about 8,000 g/mol.

55. The pharmaceutical composition according to claim 53, wherein the concentration of the polyethylene glycol is from about 20 percent (w/w) to about 99 percent (w/w) based on the total weight of the composition.

56. The pharmaceutical composition according to claim 46, wherein the composition comprises a plasticizing agent.

57. The pharmaceutical composition according to claim 56, wherein the plasticizing agent is selected from the group consisting of glycerin, propylene glycol, and sorbitol.

58. The pharmaceutical composition according to claim 46, wherein the composition comprises a surfactant.

59. A method of treating a condition for which is indicated an analgesic in animals comprising administering to an animal in need of treatment a therapeutically effective amount of the ion pair compound according to claim 1.

60. A method of treating a condition for which is indicated an anti-inflammatory agent in animals comprising administering to an animal in need of treatment a therapeutically effective amount of the ion pair compound according to claim 1.

61. A method of treating a condition for which is indicated an analgesic and an anti-inflammatory agent in animals comprising administering to an animal in need of treatment a therapeutically effective amount of the ion pair compound according to claim 1.

62. The method according to claim 59 wherein the animal is a mammal.

63. The method according to claim 62 wherein the mammal is a human.

64. The method according to claim 60 wherein the animal is a mammal.

65. The method according to claim 64 wherein the mammal is a human

66. The method according to claim 61 wherein the animal is a mammal.

67. The method according to claim 66 wherein the mammal is a human.

68. A method of treating a condition for which is indicated an analgesic in animals comprising administering to an animal in need of treatment a therapeutically effective amount of the pharmaceutical composition according to claim 33.

69. A method of treating a condition for which is indicated an anti-inflammatory agent in animals comprising administering to an animal in need of treatment a therapeutically effective amount of the pharmaceutical composition according to claim 33.

70. A method of treating a condition for which is indicated an analgesic and an anti-inflammatory agent in animals comprising administering to an animal in need of treatment a therapeutically effective amount of the pharmaceutical composition according to claim 33.

71. The method according to claim 68 wherein the animal is a mammal.

72. The method according to claim 71 wherein the mammal is a human.

73. The method according to claim 69 wherein the animal is a mammal.

74. The method according to claim 73 wherein the mammal is a human.

75. The method according to claim 70 wherein the animal is a mammal.

76. The method according to claim 75 wherein the mammal is a human.

77. The method according to claim 59, wherein the condition is selected from the group consisting of arthritic disorders, gastrointestinal conditions, inflammatory conditions, pulmonary inflammation, opthalmic diseases, central nervous systems disorders, pain, fever, inflammation-related cardiovascular disorders, angiogenesis-related disorders, benign and malignant tumors, adenomatous polyps, fibrosis which occurs with radiation treatment, endometriosis, osteoporosis, dysmenorrhea, premature labor, asthma, eosinophil-related disorders, pyrexia, bone resorption, nephrotoxicity, hypotension, arthrosis, joint stiffness, kidney disease, liver disease, acute mastitis, diarrhea, colonic adenomas, bronchitis, allergic neuritis, cytomegalovirus infectivity, apoptosis, HIV-induced apoptosis, lumbago, psoriasis, eczema, acne, burns, dermatitis, ultraviolet radiation damage, allergic rhinitis, respiratory distress syndrome, and endotoxin shock syndrome.

78. The method according to claim 77, wherein the condition is an arthritic disorder.

79. The method according to claim 78, wherein the arthritic disorder is selected from the group consisting of rheumatoid arthritis and osteoarthritis.

80. The method according to claim 59, wherein the condition is primary dysmenorrhea.

81. The method according to claim 59, wherein the condition is ankylosing spondylitis.

82. The method according to claim 77, wherein the condition is an inflammatory disorder.

83. The method according to claim 82, wherein the inflammatory disorder is selected from the group consisting of tendinitis and bursitis.

84. The method according to claim 77, wherein the condition is acute gouty arthritis.

85. The method according to claim 77, wherein the condition is pain.

86. The method according to claim 85, wherein the type of pain treated is anogenital, minor arthritic, dental, topical, associated with an upper respiratory infection, general, joint, menstrual, mild, mild to moderate, acute musculo-skeletal, moderate to moderately severe, moderate to severe, muscular, neurogenic, obstetrical, ocular, oral mucosal and gingival, post operative, pre-operative, pre- and post-operative, severe, short term, urinary tract, or associated with gastric hyperacidity.

87. The method according to claim 85 wherein the type of pain is mild to moderate.

88. The method according to claim 85 wherein the type of pain is moderate to moderately severe.

89. The method according to claim 85 wherein the type of pain is moderate to severe.

90. The method according to claim 85 wherein the type of pain is severe.

91. The method according to claim 85 wherein the type of pain is selected from the group consisting of pre-operative, post operative, and pre- and post-operative.

92. The method according to claim 59, wherein the NSAID is selected from the group consisting of diclofenac, etodolac, sulindac, alclofenac, fenclofenac, diflunisal, benorylate, fosfosal, aspirin, salicylic acid, acetylsalicylic acid, ibuprofen, ketoprofen, naproxen, carprofen, fenbufen, flurbiprofen, oxaprozin, suprofen, triaprofenic acid, fenoprofen, indoprofen, piroprofen, flufenamic, mefenamic, meclofenamic, niflumic, salsalate, rolmerin, fentiazac, tilomisole, oxyphenbutazone, phenylbutazone, apazone, feprazone, sudoxicam, isoxicam, tenoxicam, piroxicam, indomethacin, meloxicam, nabumetone, tolmetin, lumiracoxib, and parecoxib.

93. The method according to claim 92, wherein the NSAID is selected from the group consisting of diclofenac, etodolac, sulindac, salicylic acid, acetylsalicylic acid, ibuprofen, ketoprofen, naproxen, lumiracoxib, and parecoxib.

94. The method according to claim 59, wherein the NSAID is a COX-2 selective inhibitor.

95. The method according to claim 94, wherein the COX-2 selective inhibitor is selected from the group consisting of etoricoxib, rofecoxib, celecoxib, valdecoxib.

96. The method according to claim 59, wherein the NSAID is a COX-LOX inhibitor.

97. The method according to claim 59, wherein the NSAID is a PLA₂ inhibitor.

98. The method according to claim 59, wherein the narcotic is propoxyphene.

99. The method according to claim 59, wherein the compound is selected from the group consisting of propoxyphene diclofenate, ketamine diclofenate, methadone diclofenate, hydrocodone diclofenate, codeine diclofenate, propoxyphene salicylate, propoxyphene acetylsalicylate propoxyphene ibuprofenate, morphine diclofenate, and oxycodone diclofenate.

100. The method according to claim 59, wherein the compound is selected from the group consisting of propoxyphene naproxenate, propoxyphene etodolate, propoxyphene ketoprofenate, propoxyphene sulindate, propoxyphene suprofenate, propoxyphene flurbiprofenate, propoxyphene tolmetinate, propoxyphene fenoprofenate, propoxyphene oxaprozinate, propoxyphene difunisalate, propoxyphene loxoprofenate,
ketamine acetylsalicylate, ketamine naproxenate, ketamine etodolate, ketamine sulindate, ketamine ketoprofenate, ketamine suprofenate, ketamine flurbiprofenate, ketamine tolmetinate, ketamine fenoprofenate, ketamine oxaprozinate, ketamine difunisalate, ketamine loxoprofenate, ketamine ibuprofenate, ketamine salicylate, ketamine indomethacinate, ketamine diclofenate,
methadone ibuprofenate, methadone acetylsalicylate, methadone salicylate, methadone indomethacinate, methadone naproxenate, methadone etodolate, methadone sulinate, methadone ketoprofenate, methadone suprofenate, methadone flurbiprofenate, methadone tolmetinate, methadone fenoprofenate, methadone oxaprozinate, methadone difunisalate, methadone loxoprofenate,
hydrocodone ibuprofenate, hydrocodone acetylsalicylate, hydrocodone salicylate, hydrocodone indomethacinate, hydrocodone naproxenate, hydrocodone etodolate, hydrocodone sulindate, hydrocodone ketoprofenate, hydrocodone suprofenate, hydrocodone flurbiprofenate, hydrocodone tolmetinate, hydrocodone fenoprofenate, hydrocodone oxaprozinate, hydrocodone difunisalate, hydrocodone loxoprofenate,
codeine ibuprofenate, codeine acetylsalicylate, codeine salicylate, codeine indomethacinate, codeine naproxenate, codeine etodolate, codeine sulindate, codeine ketoprofenate, codeine suprofenate, codeine flurbiprofenate, codeine tolmetinate, codeine fenoprofenate, codeine oxaprozinate, codeine difunisalate, codeine loxoprofenate,
morphine ibuprofenate, morphine acetylsalicylate, morphine salicylate, morphine indomethacinate, morphine naproxenate, morphine etodolate, morphine sulindate, morphine ketoprofenate, morphine suprofenate, morphine flurbiprofenate, morphine tolmetinate, morphine fenoprofenate, morphine oxaprozinate, morphine difunisalate, morphine loxoprofenate,
levorphanol ibuprofenate, levorphanol acetylsalicylate, levorphanol salicylate, levorphanol indomethacinate, levorphanol naproxenate, levorphanol etodolate, levorphanol sulindate, levorphanol ketoprofenate, levorphanol suprofenate, levorphanol flurbiprofenate, levorphanol tolmetinate, levorphanol fenoprofenate, levorphanol oxaprozinate, levorphanol difunisalate, levorphanol loxoprofenate,
oxycodone ibuprofenate, oxycodone acetylsalicylate, oxycodone salicylate, oxycodone indomethacinate, oxycodone naproxenate, oxycodone etodolate, oxycodone sulindate, oxycodone ketoprofenate, oxycodone suprofenate, oxycodone flurbiprofenate, oxycodone tolmetinate, oxycodone fenoprofenate, oxycodone oxaprozinate, oxycodone difunisalate, oxycodone loxoprofenate
fentanyl naproxenate, fentanyl etodolate, fentanyl ketoprofenate, fentanyl sulindate, fentanyl suprofenate, fentanyl flurbiprofenate, fentanyl tolmetinate, fentanyl fenoprofenate, fentanyl oxaprozinate, fentanyl difunisalate, fentanyl loxoprofenate,
meperidine naproxenate, meperidine etodolate, meperidine ketoprofenate, meperidine sulindate, meperidine suprofenate, meperidine flurbiprofenate, meperidine tolmetinate, meperidine fenoprofenate, meperidine oxaprozinate, meperidine difunisalate, meperidine loxoprofenate,
hydromorphone naproxenate, hydromorphone etodolate, hydromorphone ketoprofenate, hydromorphone sulindate, hydromorphone suprofenate, hydromorphone flurbiprofenate, hydromorphone tolmetinate, hydromorphone fenoprofenate, hydromorphone oxaprozinate, hydromorphone difunisalate, hydromorphone loxoprofenate,
oxymorphone naproxenate, oxymorphone etodolate, oxymorphone ketoprofenate, oxymorphone sulindate, oxymorphone suprofenate, oxymorphone flurbiprofenate, oxymorphone tolmetinate, oxymorphone fenoprofenate, oxymorphone oxaprozinate, oxymorphone difunisalate, oxymorphone loxoprofenate,
dihydrocodeine naproxenate, dihydrocodeine etodolate, dihydrocodeine ketoprofenate, dihydrocodeine sulindate, dihydrocodeine suprofenate, dihydrocodeine flurbiprofenate, dihydrocodeine tolmetinate, dihydrocodeine fenoprofenate, dihydrocodeine oxaprozinate, dihydrocodeine difunisalate, and dihydrocodeine loxoprofenate.

101. The method according to claim 99, wherein the compound is propoxyphene diclofenate.

102. The method according to claim 60, wherein the condition is selected from the group consisting of arthritic disorders, gastrointestinal conditions, inflammatory conditions, pulmonary inflammation, opthalmic diseases, central nervous systems disorders, pain, fever, inflammation-related cardiovascular disorders, angiogenesis-related disorders, benign and malignant tumors, adenomatous polyps, fibrosis which occurs with radiation treatment, endometriosis, osteoporosis, dysmenorrhea, premature labor, asthma, eosinophil-related disorders, pyrexia, bone resorption, nephrotoxicity, hypotension, arthrosis, joint stiffness, kidney disease, liver disease, acute mastitis, diarrhea, colonic adenomas, bronchitis, allergic neuritis, cytomegalovirus infectivity, apoptosis, HIV-induced apoptosis, lumbago, psoriasis, eczema, acne, burns, dermatitis, ultraviolet radiation damage, allergic rhinitis, respiratory distress syndrome, and endotoxin shock syndrome.

103. The method according to claim 102, wherein the condition is an arthritic disorder.

104. The method according to claim 103, wherein the arthritic disorder is selected from the group consisting of rheumatoid arthritis and osteoarthritis.

105. The method according to claim 60, wherein the condition is primary dysmenorrhea.

106. The method according to claim 60, wherein the condition is ankylosing spondylitis.

107. The method according to claim 102, wherein the condition is an inflammatory disorder.

108. The method according to claim 107, wherein the inflammatory disorder is selected from the group consisting of tendinitis and bursitis.

109. The method according to claim 102, wherein the condition is acute gouty arthritis.

110. The method according to claim 102, wherein the condition is pain.

111. The method according to claim 110, wherein the type of pain treated is anogenital, minor arthritic, dental, topical, associated with an upper respiratory infection, general, joint, menstrual, mild, mild to moderate, acute musculo-skeletal, moderate to moderately severe, moderate to severe, muscular, neurogenic, obstetrical, ocular, oral mucosal and gingival, post operative, pre-operative, pre- and post-operative, severe, short term, urinary tract, or associated with gastric hyperacidity.

112. The method according to claim 110 wherein the type of pain is mild to moderate.

113. The method according to claim 110 wherein the type of pain is moderate to moderately severe.

114. The method according to claim 110 wherein the type of pain is moderate to severe.

115. The method according to claim 110 wherein the type of pain is severe.

116. The method according to claim 110 wherein the type of pain is selected from the group consisting of pre-operative, post operative, and pre- and post-operative.

117. The method according to claim 60, wherein the NSAID is selected from the group consisting of diclofenac, etodolac, sulindac, alclofenac, fenclofenac, diflunisal, benorylate, fosfosal, aspirin, salicylic acid, acetylsalicylic acid, ibuprofen, ketoprofen, naproxen, carprofen, fenbufen, flurbiprofen, oxaprozin, suprofen, triaprofenic acid, fenoprofen, indoprofen, piroprofen, flufenamic, mefenamic, meclofenamic, niflumic, salsalate, rolmerin, fentiazac, tilomisole, oxyphenbutazone, phenylbutazone, apazone, feprazone, sudoxicam, isoxicam, tenoxicam, piroxicam, indomethacin, meloxicam, nabumetone, tolmetin, lumiracoxib, and parecoxib.

118. The method according to claim 117, wherein the NSAID is selected from the group consisting of diclofenac, etodolac, sulindac, salicylic acid, acetylsalicylic acid, ibuprofen, ketoprofen, naproxen, lumiracoxib, and parecoxib.

119. The method according to claim 60, wherein the NSAID is a COX-2 selective inhibitor.

120. The method according to claim 119, wherein the COX-2 selective inhibitor is selected from the group consisting of etoricoxib, rofecoxib, celecoxib, valdecoxib.

121. The method according to claim 60, wherein the NSAID is a COX-LOX inhibitor.

122. The method according to claim 60, wherein the NSAID is a PLA₂ inhibitor.

123. The method according to claim 60, wherein the narcotic is propoxyphene.

124. The method according to claim 60, wherein the compound is selected from the group consisting of propoxyphene diclofenate, ketamine diclofenate, methadone diclofenate, hydrocodone diclofenate, codeine diclofenate, propoxyphene salicylate, propoxyphene acetylsalicylate propoxyphene ibuprofenate, morphine diclofenate, and oxycodone diclofenate.

125. The method according to claim 60, wherein the compound is selected from the group consisting of propoxyphene naproxenate, propoxyphene etodolate, propoxyphene ketoprofenate, propoxyphene sulindate, propoxyphene suprofenate, propoxyphene flurbiprofenate, propoxyphene tolmetinate, propoxyphene fenoprofenate, propoxyphene oxaprozinate, propoxyphene difunisalate, propoxyphene loxoprofenate,
ketamine acetylsalicylate, ketamine naproxenate, ketamine etodolate, ketamine sulindate, ketamine ketoprofenate, ketamine suprofenate, ketamine flurbiprofenate, ketamine tolmetinate, ketamine fenoprofenate, ketamine oxaprozinate, ketamine difunisalate, ketamine loxoprofenate, ketamine ibuprofenate, ketamine salicylate, ketamine indomethacinate, ketamine diclofenate,
methadone ibuprofenate, methadone acetylsalicylate, methadone salicylate, methadone indomethacinate, methadone naproxenate, methadone etodolate, methadone sulinate, methadone ketoprofenate, methadone suprofenate, methadone flurbiprofenate, methadone tolmetinate, methadone fenoprofenate, methadone oxaprozinate, methadone difunisalate, methadone loxoprofenate,
hydrocodone ibuprofenate, hydrocodone acetylsalicylate, hydrocodone salicylate, hydrocodone indomethacinate, hydrocodone naproxenate, hydrocodone etodolate, hydrocodone sulindate, hydrocodone ketoprofenate, hydrocodone suprofenate, hydrocodone flurbiprofenate, hydrocodone tolmetinate, hydrocodone fenoprofenate, hydrocodone oxaprozinate, hydrocodone difunisalate, hydrocodone loxoprofenate,
codeine ibuprofenate, codeine acetylsalicylate, codeine salicylate, codeine indomethacinate, codeine naproxenate, codeine etodolate, codeine sulindate, codeine ketoprofenate, codeine suprofenate, codeine flurbiprofenate, codeine tolmetinate, codeine fenoprofenate, codeine oxaprozinate, codeine difunisalate, codeine loxoprofenate,
morphine ibuprofenate, morphine acetylsalicylate, morphine salicylate, morphine indomethacinate, morphine naproxenate, morphine etodolate, morphine sulindate, morphine ketoprofenate, morphine suprofenate, morphine flurbiprofenate, morphine tolmetinate, morphine fenoprofenate, morphine oxaprozinate, morphine difunisalate, morphine loxoprofenate,
levorphanol ibuprofenate, levorphanol acetylsalicylate, levorphanol salicylate, levorphanol indomethacinate, levorphanol naproxenate, levorphanol etodolate, levorphanol sulindate, levorphanol ketoprofenate, levorphanol suprofenate, levorphanol flurbiprofenate, levorphanol tolmetinate, levorphanol fenoprofenate, levorphanol oxaprozinate, levorphanol difunisalate, levorphanol loxoprofenate,
oxycodone ibuprofenate, oxycodone acetylsalicylate, oxycodone salicylate, oxycodone indomethacinate, oxycodone naproxenate, oxycodone etodolate, oxycodone sulindate, oxycodone ketoprofenate, oxycodone suprofenate, oxycodone flurbiprofenate, oxycodone tolmetinate, oxycodone fenoprofenate, oxycodone oxaprozinate, oxycodone difunisalate, oxycodone loxoprofenate
fentanyl naproxenate, fentanyl etodolate, fentanyl ketoprofenate, fentanyl sulindate, fentanyl suprofenate, fentanyl flurbiprofenate, fentanyl tolmetinate, fentanyl fenoprofenate, fentanyl oxaprozinate, fentanyl difunisalate, fentanyl loxoprofenate,
meperidine naproxenate, meperidine etodolate, meperidine ketoprofenate, meperidine sulindate, meperidine suprofenate, meperidine flurbiprofenate, meperidine tolmetinate, meperidine fenoprofenate, meperidine oxaprozinate, meperidine difunisalate, meperidine loxoprofenate,
hydromorphone naproxenate, hydromorphone etodolate, hydromorphone ketoprofenate, hydromorphone sulindate, hydromorphone suprofenate, hydromorphone flurbiprofenate, hydromorphone tolmetinate, hydromorphone fenoprofenate, hydromorphone oxaprozinate, hydromorphone difunisalate, hydromorphone loxoprofenate,
oxymorphone naproxenate, oxymorphone etodolate, oxymorphone ketoprofenate, oxymorphone sulindate, oxymorphone suprofenate, oxymorphone flurbiprofenate, oxymorphone tolmetinate, oxymorphone fenoprofenate, oxymorphone oxaprozinate, oxymorphone difunisalate, oxymorphone loxoprofenate,
dihydrocodeine naproxenate, dihydrocodeine etodolate, dihydrocodeine ketoprofenate, dihydrocodeine sulindate, dihydrocodeine suprofenate, dihydrocodeine flurbiprofenate, dihydrocodeine tolmetinate, dihydrocodeine fenoprofenate, dihydrocodeine oxaprozinate, dihydrocodeine difunisalate, and dihydrocodeine loxoprofenate.

126. The method according to claim 124, wherein the compound is propoxyphene diclofenate.

127. The method according to claim 61, wherein the condition is selected from the group consisting of arthritic disorders, gastrointestinal conditions, inflammatory conditions, pulmonary inflammation, opthalmic diseases, central nervous systems disorders, pain, fever, inflammation-related cardiovascular disorders, angiogenesis-related disorders, benign and malignant tumors, adenomatous polyps, fibrosis which occurs with radiation treatment, endometriosis, osteoporosis, dysmenorrhea, premature labor, asthma, eosinophil-related disorders, pyrexia, bone resorption, nephrotoxicity, hypotension, arthrosis, joint stiffness, kidney disease, liver disease, acute mastitis, diarrhea, colonic adenomas, bronchitis, allergic neuritis, cytomegalovirus infectivity, apoptosis, HIV-induced apoptosis, lumbago, psoriasis, eczema, acne, burns, dermatitis, ultraviolet radiation damage, allergic rhinitis, respiratory distress syndrome, and endotoxin shock syndrome.

128. The method according to claim 127, wherein the condition is an arthritic disorder.

129. The method according to claim 128, wherein the arthritic disorder is selected from the group consisting of rheumatoid arthritis and osteoarthritis.

130. The method according to claim 61, wherein the condition is primary dysmenorrhea.

131. The method according to claim 61, wherein the condition is ankylosing spondylitis.

132. The method according to claim 127, wherein the condition is an inflammatory disorder.

133. The method according to claim 132, wherein the inflammatory disorder is selected from the group consisting of tendinitis and bursitis.

134. The method according to claim 127, wherein the condition is acute gouty arthritis.

135. The method according to claim 127, wherein the condition is pain.

136. The method according to claim 135, wherein the type of pain treated is anogenital, minor arthritic, dental, topical, associated with an upper respiratory infection, general, joint, menstrual, mild, mild to moderate, acute musculo-skeletal, moderate to moderately severe, moderate to severe, muscular, neurogenic, obstetrical, ocular, oral mucosal and gingival, post operative, pre-operative, pre- and post-operative, severe, short term, urinary tract, or associated with gastric hyperacidity.

137. The method according to claim 136 wherein the type of pain is mild to moderate.

138. The method according to claim 136 wherein the type of pain is moderate to moderately severe.

139. The method according to claim 136 wherein the type of pain is moderate to severe.

140. The method according to claim 136 wherein the type of pain is severe.

141. The method according to claim 136 wherein the type of pain is selected from the group consisting of pre-operative, post operative, and pre- and post-operative.

142. The method according to claim 61, wherein the NSAID is selected from the group consisting of diclofenac, etodolac, sulindac, alclofenac, fenclofenac, diflunisal, benorylate, fosfosal, aspirin, salicylic acid, acetylsalicylic acid, ibuprofen, ketoprofen, naproxen, carprofen, fenbufen, flurbiprofen, oxaprozin, suprofen, triaprofenic acid, fenoprofen, indoprofen, piroprofen, flufenamic, mefenamic, meclofenamic, niflumic, salsalate, rolmerin, fentiazac, tilomisole, oxyphenbutazone, phenylbutazone, apazone, feprazone, sudoxicam, isoxicam, tenoxicam, piroxicam, indomethacin, meloxicam, nabumetone, tolmetin, lumiracoxib, and parecoxib.

143. The method according to claim 142, wherein the NSAID is selected from the group consisting of diclofenac, etodolac, sulindac, salicylic acid, acetylsalicylic acid, ibuprofen, ketoprofen, naproxen, lumiracoxib, and parecoxib.

144. The method according to claim 61, wherein the NSAID is a COX-2 selective inhibitor.

145. The method according to claim 94, wherein the COX-2 selective inhibitor is selected from the group consisting of etoricoxib, rofecoxib, celecoxib, valdecoxib.

146. The method according to claim 61, wherein the NSAID is a COX-LOX inhibitor.

147. The method according to claim 61, wherein the NSAID is a PLA₂ inhibitor.

148. The method according to claim 61, wherein the narcotic is propoxyphene.

149. The method according to claim 61, wherein the compound is selected from the group consisting of propoxyphene diclofenate, ketamine diclofenate, methadone diclofenate, hydrocodone diclofenate, codeine diclofenate, propoxyphene salicylate, propoxyphene acetylsalicylate propoxyphene ibuprofenate, morphine diclofenate, and oxycodone diclofenate.

150. The method according to claim 61, wherein the compound is selected from the group consisting of propoxyphene naproxenate, propoxyphene etodolate, propoxyphene ketoprofenate, propoxyphene sulindate, propoxyphene suprofenate, propoxyphene flurbiprofenate, propoxyphene tolmetinate, propoxyphene fenoprofenate, propoxyphene oxaprozinate, propoxyphene difinisalate, propoxyphene loxoprofenate,
ketamine acetylsalicylate, ketamine naproxenate, ketamine etodolate, ketamine sulindate, ketamine ketoprofenate, ketamine suprofenate, ketamine flurbiprofenate, ketamine tolmetinate, ketamine fenoprofenate, ketamine oxaprozinate, ketamine difunisalate, ketamine loxoprofenate, ketamine ibuprofenate, ketamine salicylate, ketamine indomethacinate, ketamine diclofenate,
methadone ibuprofenate, methadone acetylsalicylate, methadone salicylate, methadone indomethacinate, methadone naproxenate, methadone etodolate, methadone sulinate, methadone ketoprofenate, methadone suprofenate, methadone flurbiprofenate, methadone tolmetinate, methadone fenoprofenate, methadone oxaprozinate, methadone difinisalate, methadone loxoprofenate,
hydrocodone ibuprofenate, hydrocodone acetylsalicylate, hydrocodone salicylate, hydrocodone indomethacinate, hydrocodone naproxenate, hydrocodone etodolate, hydrocodone sulindate, hydrocodone ketoprofenate, hydrocodone suprofenate, hydrocodone flurbiprofenate, hydrocodone tolmetinate, hydrocodone fenoprofenate, hydrocodone oxaprozinate, hydrocodone difunisalate, hydrocodone loxoprofenate,
codeine ibuprofenate, codeine acetylsalicylate, codeine salicylate, codeine indomethacinate, codeine naproxenate, codeine etodolate, codeine sulindate, codeine ketoprofenate, codeine suprofenate, codeine flurbiprofenate, codeine tolmetinate, codeine fenoprofenate, codeine oxaprozinate, codeine difunisalate, codeine loxoprofenate,
morphine ibuprofenate, morphine acetylsalicylate, morphine salicylate, morphine indomethacinate, morphine naproxenate, morphine etodolate, morphine sulindate, morphine ketoprofenate, morphine suprofenate, morphine flurbiprofenate, morphine tolmetinate, morphine fenoprofenate, morphine oxaprozinate, morphine difunisalate, morphine loxoprofenate,
levorphanol ibuprofenate, levorphanol acetylsalicylate, levorphanol salicylate, levorphanol indomethacinate, levorphanol naproxenate, levorphanol etodolate, levorphanol sulindate, levorphanol ketoprofenate, levorphanol suprofenate, levorphanol flurbiprofenate, levorphanol tolmetinate, levorphanol fenoprofenate, levorphanol oxaprozinate, levorphanol difunisalate, levorphanol loxoprofenate, oxycodone ibuprofenate, oxycodone acetylsalicylate, oxycodone salicylate,
oxycodone indomethacinate, oxycodone naproxenate, oxycodone etodolate, oxycodone sulindate, oxycodone ketoprofenate, oxycodone suprofenate, oxycodone flurbiprofenate, oxycodone tolmetinate, oxycodone fenoprofenate, oxycodone oxaprozinate, oxycodone difunisalate, oxycodone loxoprofenate fentanyl naproxenate, fentanyl etodolate, fentanyl ketoprofenate, fentanyl sulindate, fentanyl suprofenate, fentanyl flurbiprofenate, fentanyl tolmetinate,
fentanyl fenoprofenate, fentanyl oxaprozinate, fentanyl difunisalate, fentanyl loxoprofenate, meperidine naproxenate, meperidine etodolate, meperidine ketoprofenate,
meperidine sulindate, meperidine suprofenate, meperidine flurbiprofenate, meperidine tolmetinate, meperidine fenoprofenate, meperidine oxaprozinate, meperidine difunisalate, meperidine loxoprofenate,
hydromorphone naproxenate, hydromorphone etodolate, hydromorphone ketoprofenate, hydromorphone sulindate, hydromorphone suprofenate, hydromorphone flurbiprofenate, hydromorphone tolmetinate, hydromorphone fenoprofenate, hydromorphone oxaprozinate, hydromorphone difunisalate, hydromorphone loxoprofenate,
oxymorphone naproxenate, oxymorphone etodolate, oxymorphone ketoprofenate, oxymorphone sulindate, oxymorphone suprofenate, oxymorphone flurbiprofenate, oxymorphone tolmetinate, oxymorphone fenoprofenate, oxymorphone oxaprozinate, oxymorphone difunisalate, oxymorphone loxoprofenate,
dihydrocodeine naproxenate, dihydrocodeine etodolate, dihydrocodeine ketoprofenate, dihydrocodeine sulindate, dihydrocodeine suprofenate, dihydrocodeine flurbiprofenate, dihydrocodeine tolmetinate, dihydrocodeine fenoprofenate, dihydrocodeine oxaprozinate, dihydrocodeine difunisalate, and dihydrocodeine loxoprofenate.

151. The method according to claim 149, wherein the compound is propoxyphene diclofenate.

152. A process for preparing the ion pair compound according to claim 1, comprising the steps of contacting a salt of the formula {[narcotic]⁺}ₓX^{-x} with a salt of the formula [A]⁻B⁺,
wherein
x is 1,2, or 3;
X is an anion with a charge of −x; and
B⁺ is a cation.

153. The process according to claim 152, further comprising dissolving {[narcotic]⁺}ₓX^{-x} and [A]⁻B⁺ in separate volumes of the same solvent or different solvents prior to the contacting.

154. The process according to claim 153, wherein the ion pair compound of general formula I is sparingly soluble or insoluble in the solvent or different solvents, such that the contacting results in the precipitation of the ion pair compound.

155. The process according to claim 152, wherein the ion pair compound of general formula I is soluble in the solvent or different solvents.

156. The process according to claim 155, further comprising removing said solvent or solvents and isolating the ion pair compound of general formula I.

157. The process according to claim 153, further wherein the contacting occurs on a cation exchange medium.

158. The process according to claim 157, wherein the cation exchange medium is a cation exchange chromatography column.

159. The process according to claim 158, further comprising eluting the ion pair compound of general formula I from the chromatography column.

160. A composition comprising a plurality of ion pair compounds, their pharmaceutically acceptable solvates, hydrates, polymorphs, and/or isotopically labeled versions thereof according to claim 1.
